(19) United States
(12) Patent Application Publication
(10) Pub. No.: US 2006/0134068 A1
(43) Pub. Date: Jun. 22, 2006

(54) Treatment of cancer by simultaneous inhibiton of BRAF and restoration or mimicry of p16INK4A activity

(75) Inventor: Jianli Dong, New York, NY (US)

Correspondence Address: DARBY & DARBY P.C., P. O. BOX 5257, NEW YORK, NY, 10150-5257, US

(73) Assignee: Mount Sinai School of Medicine of New York University, New York, NY (US)

(21) Appl. No.: 11/270,796

(22) Filed: Nov. 8, 2005

Related U.S. Application Data
(60) Provisional application No. 60/626,345, filed on Nov. 9, 2004.

(51) Int. Cl. A61K 48/00

(52) U.S. Cl. 424/93.2; 514/44

(57) ABSTRACT

Provided is a method for inhibiting growth of a tumor cell which comprises oncogenically activated BRAF and defective p16^{INK4A} by inhibiting activated BRAF and restoring functional p16^{INK4A}. Also provided is a method for sensitizing the foregoing tumor cell to cytotoxic or cytostatic effect of a chemotherapeutic agent or radiation by further contacting the cell with such an agent. Also provided is a method for treating cancer, especially melanoma, by simultaneously inhibiting expression or activity of activated BRAF and restoring or mimicking the activity of wild-type p16^{INK4A}.

This application claims priority from U.S. Provisional Application Ser. No. 60/626,345, filed Nov. 9, 2004, the disclosure of which is herein incorporated by reference in its entirety.

### FIELD OF THE INVENTION

The present invention demonstrates that some tumors harbor both BRAF and p16^{INK4A} lesions, which lead to defective ERK and CDK/RB pathway signaling. The present invention provides a method for treating cancer, especially melanoma, by simultaneously inhibiting expression or activity of BRAF and restoring or mimicking the activity of wild-type p16^{INK4A}.

### BACKGROUND

### Cancer and Melanoma

Cancer is the second leading cause of death in the United States, surpassed only by heart disease. In its report, Cancer Facts & Figures 2004, the American Cancer Society (ACS) predicts that 1.4 million new cases will be diagnosed in 2004, a 7% increase from 2003. Of the newly diagnosed patients, 59,350 will have skin cancer (not including basal and squamous cell carcinomas). Melanoma is the most lethal of human skin malignancies, comprising more than 77% of skin cancer deaths, with 55,100 new cases projected for the 2004 calendar year in the United States. According to the ACS report, 7,910 Americans will die from melanoma in 2004—a 4% increase over 2003 and an 8% increase over 2002. At the current rate, 1 in 67 Americans will develop invasive melanoma in his or her lifetime, a two-thousand percent increase from 1930.

Melanoma is characterized by uncontrolled and aggressive growth of melanocytes: pigment-producing tanning cells. It is believed that melanoma progresses through histologically recognizable sequential steps including a radial growth phase (RGP), a vertical growth phase (VGP), and metastatic phase (Elder, Acta Oncol. 38:535-47 (1999)). In RGP, neoplastic cells are confined to the epidermis or with microinvasion into the dermis. In more advanced VGP, cancer cells expand in the dermis and generate tumor nodules with a high potential for metastatic spread. In the metastatic phase, cancer cells disseminate to lymph nodes or distant organs (Clark and Tucker, Hum. Pathol. 29:8-14 (1998); Elder, supra).

When melanoma is detected in its early localized stages, the treatment of choice is surgery which is usually successful. Indeed, for localized melanoma that has not spread beyond the outer layers of the skin at the time of detection, the average five-year survival rate is 97%. About 82% of melanomas are diagnosed at a localized stage (Cancer Facts & Figures 2004).

In its advanced stages, melanoma can spread quickly to other parts of the body. For such invasive and metastatic melanomas, there is currently no effective treatment. Patients with late stage melanomas have an average five-year survival rate of only 14%, with some dying within 6-8 months after diagnosis. The aggressive growth of melanoma cells as well as their intrinsic resistance to all the standard modalities of cancer treatment account for the dismal prognosis (Soengas and Lowe, Oncogene 22:3138-51 (2003); Ivanov et al., Oncogene 22:3152-61 (2003)). Therefore, the ability to inhibit growth and overcome drug resistance is central to the efficient treatment of melanomas.

### Melanoma and Activation of BRAF

BRAF is one of three members of the RAF family, each of which encodes a serine/threonine kinase that transduces regulatory signals from RAS through MEK (MAPK kinase) to ERK. The ERK signaling pathway plays essential roles in cell proliferation, differentiation and survival (Peyssonnaux and Eychene, Biol. Cell. 93:53-62 (2001); Dent and Grant, Clin. Cancer Res. 7:775-83 (2001); English and Cobb, Trends Pharmacol. Sci. 23:40-5 (2002)). The ERK signaling pathway conveys extra- and intracellular signals to transcription factors that regulate gene expression in response to regulatory signals (Kolch, Biochem. J. 351 Pt 2:289-305 (2000); Pearson et al., Endocr. Rev. 22:153-83 (2001); Chang et al., Oncol. 22:469-80 (2003)).

Oncogenic BRAF mutations have been identified in about 70% of malignant melanomas (Davies et al., Nature 417:949-54 (2002)) and are implicated in the malignant growth of melanoma cells (Wellbrock et al., Cancer Res. 64:2338-42 (2004); Hingorani et al., Cancer Res. 63:5198-202 (2003)). A T1796A transversion in exon 15, which results in a V599E substitution in the BRAF kinase domain, accounts for about 90% of BRAF mutations detected in melanoma samples (Davies et al., supra). V599E refers to the change in the wild-type codon GTG (valine) to GAG (glutamate) at position 599. The V599E mutation increases BRAF kinase activity (Davies et al., supra; Dong et al., supra). BRAF oncogenic mutations lead to constitutive activation of the RAS-RAF-mitogen activated protein kinase/ERK kinase (MEK)-extracellular signal regulated kinase (ERK) signaling pathway, which is essential for cell proliferation, differentiation and survival (Davies et al., supra; Dent et al., supra; English et al., Trends Pharmacol. Sci. 23:40-5 (2002)). BRAF mutations have also been found in 70-80% of benign melanocytic nevi (Dong et al., supra; Pollack et al., Nat. Genet. 33:19-20 (2003)). These benign nevi often regress over time, thus BRAF mutations alone are insufficient to cause malignant transformation in nevus cells. It has been shown that inhibition of the ERK pathway sensitizes melanoma cells to apoptosis induced by DNA damaging agents including cisplatin and UV irradiation (Mandic et al., Melanoma Res. 11:11-9 (2001); Halaschek-Wiener et al., J. Invest. Dermatol. 120:109-15 (2003)). Activating BRAF mutations could drive cell proliferation and increase the death threshold through the ERK pathway or alternative mechanisms (Erhardt et al., Mol. Cell Biol. 19:5308-15 (1999); Satyamoorthy et al, Cancer Res. 63:756-9 (2003); Alavi et al., Science 301:94-6 (2003)), resulting in the blockage of both cytotoxic and cytostatic effects of therapeutic drugs (Dent and Grant, supra; Mandic et al., supra; Fan and Chambers, Drug Resist. Updat. 4:253-67 (2001)).

### Melanoma and p16^{INK4A} Lesions

p16^{INK4A} is a protein that binds and inhibits cyclin-dependent kinase (CDK) 4 and CDK6, and promotes cell-cycle arrest via the RB tumor suppressor pathway (Lowe et al., Curr. Opin. Genet. Dev. 13:77-83 (2003)). Most melanoma cells, but not melanocytic nevi, have defective expression of p16^{INK4A}. These lesions may include complete loss of the gene, loss of expression of the wild-type gene, partial loss of expression of the wild-type gene, expression of non-functional p16^{INK4A}, hypermethylation of the INK4 promoter, nuclear/cytoplasmic mis-localization of p16^{INK4A}, and the like. (Castellano et al., Cancer Res. 57:4868-75 (1997); Funk et al., J. Cutan. Pathol. 25:291-6 (1998); von Eggeling et al., Arch Dermatol. Res. 291:474-7 (1999); Welch et al., J. Invest. Dermatol. 117:383-4 (2001); Zhang et al., Int. J. Oncol. 21:43-8 (2002)).

### Other Cancers

In addition to melanoma, activating mutations of BRAF were reported recently in a subset of colorectal tumors, papillary thyroid carcinomas (PTC) and leukemias. A somatic mutation of BRAF, V599E, was the most common genetic change in PTCs (28 of 78; 35.8%). BRAF(V599E) mutations were unique to PTCs, and not found in any of the other types of differentiated follicular neoplasms arising from the same cell type (0 of 46) (Kimura et al., Cancer Res. 63(7):1454-7 (2003)). In colon cancer, a total of 63 exonic mutations (22%) were detected, 60 of which were V599E, and one each of D593G, G468E, and D586A (Wang et al., Cancer Res. 63(17):5209-12 (2003)). In leukemia, BRAF mutations were observed in two of 10 B-cell acute lymphocyrix leukemias (ALLs) (20%), one of 11 biphenotypic acute leukemias (9%) and one of 23 acute myeloid leukemias (AMLs) with maturation (4%). Of note, all BRAF mutations identified among acute leukemias involved codon 468 (G468A) in the G-loop domain (Leukemia, 18; 170-172 (2004)). In addition, BRAF mutations were identified in 5 non-small cell lung carcinomas (NSCLCs) (3%; one V599 and four non-V599) (Brose et al., Cancer Res. 62(23):6997-7000 (2002)), and in ovarian cancers (33 of 91, 36%) (Sieben et al., The Journal of Pathology 202:336-340 (2004)) and endometrial carcinomas (5 of 21, 24%) (Singer G. et al., J. Natl. Cancer Inst. 19;95(6):484-6 (2003)). In some cancers (e.g. ovarian cancer, Rosemary S, et al., Molecular Endocrinology 18:2570-2582), BRAF is activated by over-expression. It is unknown whether the over-expression is caused by a mutation in the promoter or other non-coding region, or through other mechanisms. In addition, in melanoma and other cancers, activation of BRAF can also be driven by RAS or other upstream signals in the RAS/RAF/MEK/ERK pathway (Davies et al., supra; Dent et al., supra; English et al., Trends Pharmacol. Sci. 23:40-5 (2002)).

### SUMMARY OF THE INVENTION

The present invention provides a method for inhibiting the growth of a tumor cell that comprises an activated BRAF gene or protein, and an absent or functionally defective p16^{INK4A}, by inhibiting expression or activity of BRAF in the tumor cell; and restoring, increasing or mimicking activity of a functional p16^{INK4A} in the tumor cell.

In one embodiment, inhibition of BRAF comprises inhibiting expression of the BRAF nucleic acid. In a preferred embodiment, inhibiting expression of BRAF nucleic acid is by RNA interference using an inhibitory RNA (RNAi) specific for BRAF.

In one embodiment of the RNA interference, the RNAi is provided in a viral vector.

In another embodiment, inhibition of BRAF comprises inhibiting expression of an endogenous BRAF polypeptide.

In a further embodiment, restoration, increase or mimicking of the activity of a functional p16^{INK4A} is achieved by introducing into the cell a nucleic acid sequence encoding a functional p16^{INK4A}.

In one embodiment, the functional INK4A nucleic acid sequence is a cDNA. In a preferred embodiment, this cDNA is provided to the cell in a viral vector.

In another embodiment, the tumor cell is a melanoma cell.

The present invention also provides a method for sensitizing a tumor cell to a chemotherapeutic agent or radiation therapy, wherein the tumor cell comprises an activated BRAF, and an abnormal INK4A gene or p16^{INK4A}, inhibiting expression or activity of mutant BRAF in the tumor cell; restoring, increasing or mimicking activity of a functional p16^{INK4A} in the tumor cell; and contacting the cell with an effective amount of a chemotherapeutic agent or radiation.

In one embodiment, inhibition of BRAF comprises inhibiting expression of a BRAF nucleic acid. In a preferred embodiment, inhibiting expression of the BRAF nucleic acid is by an RNAi specific for the BRAF.

In one embodiment of the RNA interference, the RNAi is provided in a viral vector.

In another embodiment, inhibition of BRAF comprises inhibiting expression of endogenous BRAF polypeptide.

In a further embodiment, restoration, increase of, or mimicking of the activity of a functional p16^{INK4A} is achieved by introducing into the cell a nucleic acid sequence encoding a functional p16^{INK4A}.

In one embodiment, the functional INK4A nucleic acid sequence is a cDNA. In a preferred embodiment, the cDNA is provided to the cell in a viral vector.

In another embodiment, the tumor cell is a melanoma cell.

The present invention also provides a method for treating cancer in an individual having a tumor comprising an activated BRAF, and an abnormal INK4A or p16^{INK4A}, by inhibiting expression or activity of BRAF in the tumor; and restoring, increasing or mimicking activity of a functional p16^{INK4A} in the tumor.

In one embodiment, inhibition of BRAF comprises inhibiting expression of the BRAF nucleic acid. In a preferred embodiment, inhibiting expression of BRAF nucleic acid is by RNA interference specific for mutant BRAF.

In one embodiment of the RNA interference, the RNAi is provided in a viral vector.

In another embodiment, inhibition of BRAF comprises inhibiting activity of an endogenous BRAF polypeptide.

In a further embodiment, restoration, increase or mimicking of the expression and activity of a functional p16^{INK4A} is by introducing into the cell a nucleic acid sequence encoding a functional p16^{INK4A}.

In one embodiment, the functional INK4A nucleic acid sequence is a cDNA. In a preferred embodiment, the cDNA is provided to the cell in a viral vector.

In one embodiment of the present invention, the BRAF inhibition and p16^{INK4A} restoration, increase or mimicking occurs simultaneously.

In another embodiment, the BRAF inhibition occurs prior to the p₁₆^{INK4A} restoration, increase or mimicking.

The present invention also provides combination therapy of inhibiting oncogenically activated BRAF and restoring functional p16^{INK4A} expression with customary cancer treatment such as chemotherapy, radiation, and vaccine therapy.

The present invention also provides methods for screening for mBRAF antagonists.

### BRIEF DESCRIPTION OF THE DRAWINGS

FIG. 1. FIG. 1 depicts a generally accepted model of the convergence of BRAF and p16^{INK4A} at the pRB/E2F pathway.

FIG. 2. FIG. 2 depicts a model of separate regulation of proliferation and differentiation by oncogenically activated BRAF and p16^{INK4A} in melanoma cells.

FIG. 3A-D. FIG. 3 depicts results from a colony forming assay demonstrating inhibition of tumor growth in 624Mel cells which were untreated (3A); treated with mBRAF-specific RNAi alone (3B); treated with 10 nM flavopiridol alone (3C); or treated with a combination of mBRAF-specific RNAi and 10 nM flavopiridol (3D).

FIG. 4. FIG. 4 depicts results of experiments performed to express both mBRAF RNAi and wild-type INK4A in 624Mel, and as a control, in Me11363 melanoma cells that express wild-type BRAF and p16^{INK4A}. The efficiency of BRAF inhibition and p16 expression are demonstrated via immunoblotting.

FIG. 5. FIG. 5 depicts results from an experiment demonstrating that simultaneous expression of mBRAF RNAi and wild-type INK4A significantly inhibit the growth of 624Mel cells in tissue, as measured by cell count (5A) and colony formation assay (5B). For the colony formation assay, 1×10³ 624Mel melanoma control cells, stably expressing mBRAF RNAi, INK4A, or both mBRAF RNAi and INK4A or mBRAF RNAi were plated in triplicate in 35 mm diameter plates and grown in medium with 5% serum for 3 weeks. Colonies were then fixed, stained, and counted.

FIG. 6. FIG. 6 depicts results from an experiment demonstrating that inhibition of mBRAF and restoration of wild-type INK4A synergize in promoting apoptosis. This analysis was performed by a terminal deoxynucleotidyl transferase-mediated dUTP-biotin nick end labeling of DNA fragments (TUNEL) assay.

### DETAILED DESCRIPTION

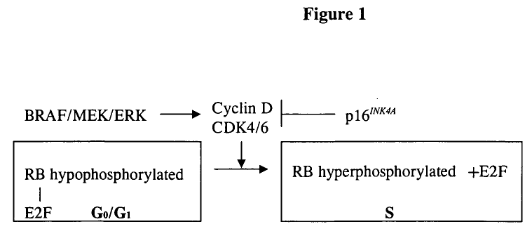

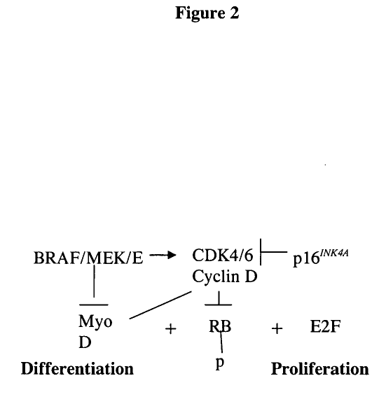

Activating BRAF mutations and defects in wild-type p16^{INK4A} expression occur at high frequencies and thus may co-exist in melanoma cells. The present invention identifies, for the first time, several melanoma cell lines that harbor both BRAF and INK4A lesions. A highly specific RNA interference (RNAi) approach was then used to inhibit expression of the T1796A “hot-spot” BRAF mutation (mBRAF). An INK4A wild-type cDNA expression construct was used to restore wild-type p16^{INK4A} expression. Inhibition of mBRAF or restoration of wild-type p16^{INK4A} both inhibited growth of melanoma cells significantly, but neither resulted in complete inhibition. Surprisingly, simultaneous inhibition of mBRAF and restoration of wild-type p16^{INK4A} in melanoma cells resulted in potent, even lethal, effects in melanoma cells. Melanogenesis, a marker of melanocyte differentiation was only induced by mBRAF inhibition suggesting non-overlapping roles of BRAF and INK4A lesions. This discovery was unexpected since it is generally believed that BRAF and INK4A lesions affect the same pathway, i.e, the MEK/ERK, cyclinD-CKD4/6, and ultimately pRB phosphorylation (see FIG. 1). These data suggest that BRAF and INK4A lesions are involved in separate regulation of proliferation and differentiation, and that they may interact synergistically in the malignant growth of melanoma cells (see FIG. 2).

### Definitions

As used herein, the term “BRAF” refers to the serine/threonine kinase BRAF polypeptide, as well as the gene encoding it. Sequences for the human BRAF gene and polypeptide are well known and can be found, for example, in SEQ ID NO: 1 and SEQ ID NO: 2, respectively.

As used herein, the term “INK4A” refers to the gene encoding the cell cycle regulator p16^{INK4A}, which is a cyclin-dependent kinase inhibitor (CDKI). CDKIs bind to cyclin-dependent kinases (CDKs), along or in complex with cyclin and inhibit cell cycle progression. p16^{INK4A} exerts its anti-proliferative effects by binding to and inhibiting the actions of primarily CDK 4 and 6. The INK4A gene is located at the chromosomal locus 9p21.

The term “p16^{INK4A}” refers to the protein encoded by the INK4A gene. A human p16^{INK4A} has nucleic acid and amino acid sequences as set forth in SEQ ID NO: 3 and SEQ ID NO: 4, respectively (GenBank Accession Nos. NM_{—}000077 and NP_{—}000068, respectively).

The term “mutant or defective INK4A” refers to an INK4A gene harboring a mutation that results in decreased levels of, or defective activity of, a functional p16^{INK4A}, e.g., a protein which lacks the ability to bind CDK4 and/or CDK6.

The term “mutant or defective p16^{INK4A}” refers to a p16^{INK4A} which is non-functional, e.g. unable to bind to or inhibit CKD4/6 or another CDK. The defect can be caused by mutations in the INK4A gene or regulatory regions, inactivation of the INK4A gene due to factors such as promoter hypermethylation, deletion of the INK4A gene, or aberrant activity or regulation of 16^{INK4A}, e.g., cellular mislocalization of p16^{INK4A} in the cytoplasm or the nucleus, or the presence of a 16^{INK4A} inhibitor.

The term “mimicking or mimicry of p16^{INK4} activity” refers to an agent that is a functional equivalent of p16^{INK4A} with respect to) its role in cell cycle inhibition. For example, such a functional equivalent may be a molecule that binds to and inhibits CDK4 and/or CDK6. A solution structure of p16^{INK4A} binding to CDK4 is known (Byeon et al., Mol. Cell. 1: 421-31 (1998)). From this structure, a fragment of 58 amino acid residues at the N-terminus of CKD4 has been identified as critical for p16^{INK4A} binding. Computer “docking” software (e.g., DOCK, INSIGHT II, APROPOS) can be used by those skilled in the art to screen compound databases or rationally design molecules that will bind to this region and mimic p16^{INK4A} activity.

The term “mBRAF” refers to a mutant BRAF gene and protein which renders the BRAF protein oncogenic by leading to constitutive activation of the RAS-RAF-mitogen activated protein kinase/ERK kinase(MEK)/extracellular signal regulated kinase (ERK) signaling pathway. Activating BRAF may also have functions other than activating the ERK pathway. In one embodiment, the mBRAF refers to a BRAF nucleic acid which has the mutation of thymine to adenine at base pair of SEQ ID NO: 1 (T1796A), leading to constitutive activation of the above signaling pathway. The designation “mBRAF” herein also will refer either to a mutated BRAF gene or a mutated BRAF polypeptide, depending on the context. In one embodiment, the mBRAF refers to a BRAF nucleic acid which has the mutation of thymine to adenine at base pair of SEQ ID NO: 1 (T1796A), leading to constitutive activation of the above signaling pathway. BRAF also has ERK pathway independent activities that may be affected by the mutation.

The term “oncogenically activated BRAF” refers to the presence of increased expression of, or activity of, a BRAF gene or protein. This increase in expression or activity can result from a mutation(s) in BRAF, as described above, which constitutively activates the MEK/ERK or other pathways, or from a defect resulting in a detectable increase in BRAF expression or activity in a cancer cell or potentially cancerous cell compared to a non-cancerous or non-potentially cancerous cell. In addition to mutations, such increased expression and/or activity may result from amplification of a wild-type BRAF nucleic acid, overexpression of a wild-type BRAF protein, e.g., by aberrant regulation of the BRAF regulatory region such as the promoter, overexpression or activation of BRAF due to aberrant regulation of an upstream regulator (e.g, RAS mutation, or inhibition of a BRAF inhibitor) or by stabilization of BRAF.

The term “RNA interference” or “RNAi” refers to the ability of double stranded RNA (dsRNA) to suppress the expression of a specific gene of interest in a homology-dependent manner. It is currently believed that RNA interference acts post-transcriptionally by targeting mRNA molecules for degradation. For reviews, see Bosner and Labouesse, Nature Cell Biol. 2: E31-E36 (2000); and Sharp and Zamore, Science 287: 2431-2433 (2000). RNAi is described further below.

RNAi molecules include small interfering RNAs (siRNAs) and short hairpin RNAs (shRNAs). These RNAi molecules comprise sequences that are complementary to, and therefore specific for, a segment of the sequence of the target locus. The term “RNAi” also encompasses the expression constructs used for intracellular synthesis of siRNAs and shRNAs.

The term “subject” as used herein refers to a mammal (e.g., a rodent such as a mouse or a rat, a pig, a primate, or companion animal (e.g., dog or cat, etc.)). In particular, the term refers to humans.

As used herein, the term “isolated” means that the material being referred to has been removed from the environment in which it is naturally found, and is characterized to a sufficient degree to establish that it is present in a particular sample. Such characterization can be achieved by any standard technique, e.g., sequencing, hybridization, immunoassay, functional assay, expression, size determination, or the like. Thus, a biological material can be “isolated” if it is free of cellular components, i.e., components of the cells in which the material is found or produced in nature. For nucleic acid molecules, an isolated nucleic acid molecule or isolated polynucleotide molecule, or an isolated oligonucleotide, can be e.g., a PCR product, an mRNA transcript, a cDNA or RNA molecule, or a restriction fragment. A nucleic acid molecule excised from the chromosome that it is naturally a part of is considered to be isolated. Such a nucleic acid molecule may or may not remain joined to regulatory, or non-regulatory, or non-coding regions, or to other regions located upstream or downstream of the gene when found in the chromosome. Nucleic acid molecules that have been spliced into vectors such as plasmids, cosmids, artificial chromosomes, phages and the like are considered isolated. In a particular embodiment, an e.g., BRAF-encoding nucleic acid or functional INK4A nucleic acid spliced into a recombinant vector, and/or transfected or infected into a host cell, is considered to be “isolated.”

Isolated nucleic acid molecules of the present invention do not encompass uncharacterized clones in man-made genomic or other libraries.

An isolated material may or may not be “purified.” The term “purified” as used herein refers to a material (e.g., a nucleic acid molecule or a protein) that has been isolated under conditions that detectably reduce or eliminate the presence of other contaminating materials. Contaminants may or may not include native materials from which the purified material has been obtained. A purified material preferably contains less than about 90%, less than about 75%, less than about 50%, less than about 25%, less than about 10%, less than about 5% or less than about 2% by weight of other components with which it was originally associated.

Methods for purification are well-known in the art. For example, nucleic acids or polynucleotide molecules can be purified by precipitation, chromatography (including preparative solid phase chromatography, and triple helix chromatography), ultracentriftigation, oligonucleotide hybridization and other means. Polypeptides can be purified by various methods including, without limitation, preparative disc-gel electrophoresis, isoelectric focusing, HPLC, reverse-phase HPLC, gel filtration, affinity chromatography, ion exchange and partition chromatography, precipitation and salting-out chromatography, extraction, and counter-current distribution. Cells can be purified by various techniques, including centrifugation, matrix separation (e.g., nylon wool separation), panning and other immunoselection techniques, depletion (e.g., complement depletion of contaminating cells), and cell sorting (e.g., fluorescence activated cell sorting (FACS)). Other purification methods are possible. The term “substantially pure” indicates the highest degree of purity that can be achieved using conventional purification techniques currently known in the art. In the context of analytical testing of the material, “substantially free” means that contaminants, if present, are below the limits of detection using current techniques, or are detected at levels that are low enough to be acceptable for use in the relevant art, for example, no more than about 2-5% (w/w). Accordingly, with respect to the purified material, the term “substantially pure” or “substantially free” means that the purified material being referred to is present in a composition where it represents 95% (w/w) or more of the weight of that composition. Purity can be evaluated by chromatography, gel electrophoresis, immunoassay, composition analysis, biological assay, or any other appropriate method known in the art.

The terms “wt,” “wt,” or “wt activity” refers to a normal or functional nucleotide sequence or a normal or functional sequence, structure, localization or activity of a protein, e.g., p16^{INK4A}. Such functionality can be tested by any means known to establish functionality of a protein. Certain attributes of a functional protein may not correspond to those of the actual in vivo wt structure, sequence and/or function, but nevertheless are aggregate surrogates of the wt structure, sequence, and/or functionality or wt behavior in such tests as are known in the art. This is an acceptable consequence of protein restoration techniques of the invention and such aggregate surrogates are thus also referred to as “functional.”

An “antagonist” is one type of modulator, and includes an agent that reduces expression or activity, or inhibits expression or activity, of e.g., a BRAF nucleic acid or polypeptide. Examples of antagonists of the BRAF nucleic acids or polypeptides of the present invention include without limitation small molecules, polypeptides, peptides, antibodies, antisense nucleic acids, ribozymes, and especially RNAi oligonucleotides. BRAF antagonists may be specific to a mutated form of BRAF, specific to wt BRAF, or may be general to both.

As used herein, the term “antibody” includes, but is not limited to, antibodies and binding fragments thereof, that specifically bind to an antigen, e.g., a BRAF polypeptide. Suitable antibodies may be polyclonal (e.g., sera or affinity purified preparations), monoclonal, or recombinant. Examples of useful fragments include separate heavy chains, light chains, Fab, F(ab′)2, Fabc, and Fv fragments. Fragments can be produced by enzymatic or chemical separation of intact immunoglobulins or by recombinant DNA techniques. Fragments may be expressed in the form of phage-coat fusion proteins (see, e.g., International PCT Publication Nos. WO 91/17271, WO 92/01047, and WO 92/06204). Typically, the antibodies, fragments, or similar binding agents bind a specific antigen with an affinity of at least 107, 108, 109, or 1010 M-1.

A “test compound” is a molecule that can be tested for its ability to act as a modulator of a gene or gene product. Test compounds can be selected without limitation from small inorganic and organic molecules (i.e., those molecules of less than about 2 kD, and more preferably less than about 1 kD in molecular weight), polypeptides (including native ligands, antibodies, antibody fragments, and other immunospecific molecules), oligonucleotides, polynucleotide molecules, and derivatives thereof. In various embodiments of the present invention, a test compound is tested for its ability to modulate the expression of a mammalian BRAF-encoding nucleic acid or BRAF protein or to bind to a mammalian BRAF protein. A compound that modulates a nucleic acid or protein of interest is designated herein as a “candidate compound” or “lead compound” suitable for further testing and development. Candidate compounds include, but are not necessarily limited to, the functional categories of agonist and antagonist.

The term “detectable change” as used herein in relation to an expression level of a gene or gene product (e.g., BRAF, p16^{INK4A}) means any biologically significant change (e.g., increase or decrease) and preferably at least a 1.5-fold change as measured by any available technique such as hybridization, quantitative PCR, or immunoblotting.

As used herein, the term “specific binding” refers to the ability of one molecule, typically an antibody, polynucleotide, polypeptide, or a small molecule ligand to contact and associate with another specific molecule, even in the presence of many other diverse molecules. “Immunospecific binding” refers to the ability of an antibody to specifically bind to (or to be “specifically immunoreactive with”) its corresponding antigen.

The term “about” means within an acceptable error range for the particular value as determined by one of ordinary skill in the art, which will depend in part on how the value is measured or determined, i.e., the limitations of the measurement system. For example, “about” can mean within an acceptable standard deviation, per the practice in the art. Alternatively, “about” can mean a range of up to ±20%, preferably up to ±10%, more preferably up to ±15%, and more preferably still up to ±1% of a given value. Alternatively, particularly with respect to biological systems or processes, the term can mean within an order of magnitude, preferably within 2-fold, of a value. Where particular values are described in the application and claims, unless otherwise stated, the term “about” is implicit and in this context means within an acceptable error range for the particular value.

Molecular Biology Definitions. In accordance with the present invention there may be employed conventional molecular biology, microbiology, and recombinant DNA techniques within the skill of the art. Such techniques are explained fully in the literature (see, e.g., SAMBROOK, FRITSCH & MANIATIS, MOLECULAR CLONING: A LABORATORY MANUAL (2d Ed., Cold Spring Harbor Laboratory Press, Cold Spring Harbor, N.Y. (1989) (herein “Sambrook et al., 1989”); DNA CLONING: A PRACTICAL APPROACH, Volumes I and II (D. N. Glover ed.) (1985); OLIGONUCLEOTIDE SYNTHESIS (M. J. Gait ed.) (1984); NUCLEIC ACID HYBRIDIZATION (B. D. Hames & S. J. Higgins eds.) (1985); TRANSCRIPTION AND TRANSLATION (B. D. Hames & S. J. Higgins, eds. ) (1984); ANIMAL CELL CULTURE (R. I. Freshney, ed.) (1986); IMMOBILIZED CELLS AND ENZYMES (IRL Press) (1986); B. PERBAL, A PRACTICAL GUIDE TO MOLECULAR CLONING (1984); and F. M. AUSUBEL ET AL. (EDS.), CURRENT PROTOCOLS IN MOLECULAR BIOLOGY (John Wiley & Sons, Inc.) (1994)).

The terms “express” and “expression” mean allowing or causing the information in a gene or DNA sequence to become manifest, for example producing a protein by activating the cellular functions involved in transcription and translation of a corresponding gene or DNA sequence. A DNA sequence is expressed in or by a cell to form an “expression product” such as a protein. The expression product itself, e.g., the resulting protein, may also be said to be “expressed” by the cell. An expression product can be characterized as nuclear, cytoplasmic, membrane bound or secreted.

The term “nuclear” means something that is located inside the cell nucleus.

The term “cytoplasmic” means something that is located inside the cytoplasm.

The term “membrane bound” means something that is anchored within the cell membrane. Membrane bound expression products normally have an intracellular and/or an extracellular domain, referring to a portion of the expression product that faces the inside of the cell and the outside of the cell, respectively. A substance is “secreted” by a cell if it is is expelled, from somewhere on or inside the cell to the outside of the cell.

The terms “transfection,” “infection” or “transduction” mean the introduction of genetic material (a gene, DNA, or RNA sequence) to a cell, so that the host cell will express the introduced sequence to produce a desired substance, typically a protein or enzyme coded by the introduced gene or sequence. The introduced sequence may also be a “cloned” or “foreign” gene or sequence, may include regulatory or control sequences, such as start, stop, promoter, signal, secretion, or other sequences used by a cell's genetic machinery. The gene or sequence may include nonfunctional sequences or sequences with no known function. A host cell that receives and expresses introduced DNA or RNA has been “transfected,” “infected,” or “transduced”. The term “transfection” means the introduction of genetic material through the direct delivery of a naked or complexed DNA molecule. The term “infection” means the introduction of genetic material through the use of a recombinant virus, e.g., an adenovirus or a retrovirus. The term “transduction” refers broadly to the introduction in to a cell of genetic material, regardless of the vehicle in which the genetic material is provided. The DNA or RNA introduced to a host cell can come from any source, including cells of the same genus or species as the host cell, or cells of a different genus or species.

According to the present invention, a “transduced,” “transfected,” or “infected” cells includes cells in which nucleic acid sequences (e.g., RNAi) have been inserted by any genetic manipulation. This term also includes cells in which gene activation has occurred, using e.g., homologous recombination (see U.S. Pat. Nos. 5,733,761, and 6,565,844, to Treco et al.).

The term “host cell” means any cell of any organism. The cell can be selected, modified, transformed, grown or used or manipulated in any way for the production of a substance by the cell. For example, a host cell may be one that is manipulated to express a particular gene, a DNA or RNA sequence, a protein or an enzyme. Host cells can further be used for screening or other assays that are described infra. Host cells may be cultured in vitro or in a non-human animal (e.g., a xenograft, transgenic animal or a transiently transfected animal). For the present invention, host cells include but are not limited to melanoma cells, including cell lines. For example, host cells include the WM35, 624Mel, Mel1363 and A375 cells.

The term “expression system” means a host cell and compatible vector under suitable conditions, e.g. for the expression of a protein coded for by foreign DNA or RNA carried by the vector and introduced to the host cell. In a specific embodiment, the host cell of the present invention is a melanoma cell, and the vector is an mBRAF RNAi or a retroviral construct containing a wt INK4A.

The terms “vector,” “cloning vector,” and “expression vector” refer to recombinant constructs including, e.g., plasmids, cosmids, phages, viruses, and the like, with which a nucleic acid molecule (e.g., a p16^{INK4A}-encoding nucleic acid or mBRAF RNAi-expressing nucleic acid) can be introduced into a host cell so as to, e.g., clone (expand) the vector or express the introduced nucleic acid molecule. Vectors for gene therapy include retroviral vectors, adenoviral vectors, adeno-associated viral vectors, and herpes simplex viral vectors. All vectors may further comprise selectable markers.

Gene therapy involves replacing a defective or missing gene by introducing a functional gene into somatic cells of an individual in need, for the purpose of correcting a genetic defect. Gene therapy can be accomplished by “ex vivo” methods, in which differentiated, tumor, or somatic stem cells are removed from the individual's body, followed by the introduction of a normal copy of the defective gene into the explanted cells using a viral vector as the gene delivery vehicle. In vivo direct gene transfer technologies deliver the therapeutic gene in situ using a broad range of viral vectors, liposomes, protein DNA complexes, naked DNA and other approaches in order to achieve a therapeutic outcome. Changes in gene expression can also be achieved by means other than gene therapy. For example, de-methylation reagents can be used to remove hyper-methylation in the INK4A regulatory region. Hyper-methylation is a common way of inhibiting INK4A expression.

The terms “mutant” and “mutation” mean any change in genetic material, e.g. DNA, or any process, mechanism, or result of such a change. This includes gene mutations, in which the structure (e.g., DNA sequence) of a gene is altered, any gene or DNA arising from any mutation process, and any expression product (e.g., protein or enzyme) expressed by a modified gene or DNA sequence. For example, according to the present invention, a BRAF mutant can refer to a BRAF nucleic acid sequence having the T1796A transversion.

The terms “array” and “microarray” are used interchangeably and refer generally to any ordered arrangement (e.g., on a surface or substrate) of different molecules, referred to herein as “probes.” Each different probe of an array is capable of specifically recognizing and/or binding to a particular molecule, which is referred to herein as its “target,” in the context of arrays. Examples of typical target molecules that can be detected using microarrays include mRNA transcripts, cDNA molecules, cRNA molecules, and proteins.

The phrases “disruption of the gene,” “gene disruption,” and the like, refer to: (i) insertion of a different or defective nucleic acid sequence into an endogenous (naturally occurring) DNA sequence, e.g., into an exon or promoter region of a gene; or (ii) deletion of a portion of an endogenous DNA sequence of a gene; or (iii) combination of insertion and deletion, so as to decrease or prevent the expression of that gene or its gene product in the cell as compared to the expression of the endogenous gene sequence.

The term “restoration of a gene” includes using a different nucleic acid sequence to activate expression of a gene by replacing the endogenous gene or regulatory sequences of an endogenous gene with exogenous regulatory or coding sequences or endogenous regulatory or coding sequences from another gene in the genome. This term also includes activating gene expression by modifying the regulatory or coding sequences, such as de-methylation of hypermethylated promoters.

### BRAF Inhibition

### BRAF Antagonists

It is expected that antagonists of BRAF will be effective for use in the methods of the present invention, e.g., to treat cancer by inhibiting oncogenically activated BRAF. Known antagonists of BRAF include a RAF inhibitor L-779,450 (Merck) and BAY 43-9006 (Bayer). In addition, in 2004, Astex Technology, the fragment-based drug discovery company, together with The Wellcome Trust, the Institute of Cancer Research and Cancer Research Technology are collaborating to identify of novel drugs to inhibit BRAF.

The present invention also contemplates the identification of compounds that modulate BRAF expression and/or activity. Such compounds are useful, e.g., for inhibiting (i.e., antagonizing) BRAF expression according to the method of the present invention.

Compounds that inhibit BRAF or mBRAF expression or activity may be readily identified using known screening methods. In one embodiment, compounds identified by the screening methods bind specifically to a BRAF nucleic acid or polypeptide. Compounds identified by the present method may antagonize BRAF, as well as a related downstream biological effect (e.g., inhibit the phosphorylation of MEK and ERK) that is associated with constitutive BRAF activity.

In vivo or cell culture assays may be used to determine whether a test compound functions as an antagonist to inhibit BRAF in cells. For instance, cell culture assays may be used to measure a test compound's ability to modulate an activity, such as detecting inhibition of endogenous phospho-MEK levels, or increase sensitivity to chemotherapy, in tumor cells treated with a test compound. Such assays generally comprise contacting a cell that expresses BRAF or mBRAF with a test compound and comparing it to control cells not contacted with the test compound.

In one non-limiting embodiment, the response of the cell treated with the test compound can be compared to a control cell that has not been treated with the test compound. Cell assays include those utilizing conventional, reporter gene-based assays, among others.

### Targeted Alteration of BRAF

Based on the present disclosure, genetic constructs can be prepared for use in disabling or otherwise otherwise inactivating an oncogenic BRAF (including mutation, over-expression, etc.). For example, the BRAF or mBRAF gene can be mutated using an appropriately designed genetic construct in combination with genetic techniques currently known or to be developed in the future. In another instance, the BRAF or mBRAF gene can be mutated using a genetic construct that functions to: (i) delete all or a portion of the coding sequence or regulatory sequence of the BRAF or mBRAF gene; (ii) replace all or a portion of the coding sequence or regulatory sequence of the BRAF or mBRAF gene with a different nucleotide sequence; (iii) insert into the coding sequence or regulatory sequence of the mBRAF gene one or more nucleotides, or an oligonucleotide molecule, or polynucleotide molecule, which can comprise a nucleotide sequence from the same species or from a heterologous source; or (iv) carry out some combination of (i), (ii) and (iii). In addition, BRAF or mBRAF expression could be silenced using chemical means, including but not limited to promoter hyper-methylation.

In a preferred embodiment, the disruption serves to partially or completely disable the activated BRAF, or partially or completely disable the activated BRAF protein. In this context, an activated BRAF gene or protein is considered to be partially or completely disabled if either no protein product is made (for example, where the gene is deleted), or a protein product is made that can no longer carry out its oncogenic function or can no longer be transported to its normal cellular location, or a protein product is made that carries out its oncogenic function but at a significantly reduced level.

In a non-limiting embodiment, a genetic construct of the present invention is used to inhibit an activated BRAF gene by replacement of at least a portion of the coding or regulatory sequence of the mutant gene with a different nucleotide sequence, e.g., a wt coding sequence or mutated regulatory region, or portion thereof. A wt BRAF gene sequence for use in such a genetic construct can be produced by any of a variety of known methods, including by use of error-prone PCR, or by cassette mutagenesis. For example, oligonucleotide-directed mutagenesis can be employed to alter the coding or regulatory sequence of a BRAF gene in a defined way, e.g., to introduce a frame-shift or a termination codon at a specific point within the sequence. A mutated nucleotide sequence for use in the genetic construct of the present invention can be prepared by insertion into the coding or regulatory (e.g., promoter) sequence of one or more nucleotides, oligonucleotide molecules or polynucleotide molecules, or by replacement of a portion of the coding sequence or regulatory sequence with one or more different nucleotides, oligonucleotide molecules or polynucleotide molecules. Such oligonucleotide molecules or polynucleotide molecules can be obtained from any naturally occurring source or can be synthetic. The inserted sequence can serve simply to disrupt the reading frame of the BRAF gene.

Mutations to produce modified cells, tissues, and animals that are useful in practicing the present invention can occur anywhere in the BRAF gene, including the open reading frame, the promoter or other regulatory region, or any other portion of the sequence that naturally comprises the gene or ORF. Such cells include ones that the oncogenically activated mutant or wt BRAF is inhibited in the expression or activity.

Alternatively, a genetic construct can comprise nucleotide sequences that naturally flank the BRAF gene or ORF in situ, with only a portion or no nucleotide sequences from the actual coding region of the gene itself. Such a genetic construct can be useful to delete the entire BRAF gene or ORF.

Methods for carrying out homologous gene replacement are known in the art. For gene targeting through homologous recombination, the genetic construct is preferably a plasmid, either circular or linearized, comprising a wt BRAF nucleotide sequence as described above. In a non-limiting embodiment, at least about 200 nucleotides of the wt sequence are used to specifically direct the genetic construct to the particular targeted mBRAF gene for homologous recombination, although shorter lengths of nucleotides may also be effective. In addition, the plasmid preferably comprises an additional nucleotide sequence encoding a reporter gene product or other selectable marker constructed so that it will insert into the genome in operative association with the regulatory element sequences of the native mBRAF gene to be disrupted. Reporter genes that can be used in practicing the invention are known in the art, and include those encoding CAT, green fluorescent protein, and (β-galactosidase, among others). Nucleotide sequences encoding selectable markers are also known in the art, and include those that encode gene products conferring resistance to antibiotics or anti-metabolites, or that supply an auxotrophic requirement.

In view of the present disclosure, methods that can be used for creating the genetic constructs of the present invention will be apparent, and can include in vitro recombinant techniques, synthetic techniques, and in vivo genetic recombination, as described, among other places, in Ausubel et al. (1989), supra; Sambrook et al. (1989), supra; Innis et al. (1995), supra; and Erlich (1992), supra.

Mammalian cells can be transfected or infected with a genetic construct of the present invention in accordance with known techniques, e.g., by electroporation. Selection of transformants can be carried out using standard techniques, such as by selecting for cells expressing a selectable marker associated with the construct. Identification of transfected or infected in which a successful recombination event has occurred and the particular target gene has been disabled can be carried out by genetic analysis, such as by PCR analysis, Southern blot analysis, or by Northern analysis to detect a lack or presence of mRNA transcripts encoding the particular protein, or by the appearance of cells lacking or expressing the particular protein, as determined, e.g., by immunological analysis, or by the appearance of the marker introduced, or some combination thereof.

RNA Interference (RNAi). It has recently been demonstrated that expression of selected genes can be suppressed in human cells by transfecting with exogenous, short RNA duplexes where one strand corresponds to a target region of the mRNA, i.e., EST of interest (Elbashir et al., Nature 411:494-498 (2001)). RNA interference commonly involves the use of double-stranded RNAs (dsRNAs) that are greater than 500 bp; however, it can also be mediated through small interfering RNAs (siRNAs) or small hairpin RNAs (shRNAs), which can be 10 or more nucleotides in length and are typically greater than 18 nucleotides in length. The siRNA molecules are typically greater than 19 duplex nucleotides, and upon entry into the cell, siRNA causes the degradation of single-stranded RNAs (ssRNAs) of identical sequences, including endogenous mRNAs. siRNA is more potent than standard anti-sense technology since it acts through a catalytic mechanism.

The RNAi to be used in the methods of the present invention are short double stranded nucleic acid duplexes comprising complementary single stranded nucleic acid molecules. In preferred embodiments, the siRNAs are short double stranded RNAs comprising annealed complementary single strand RNAs. However, the invention also encompasses embodiments in which the siRNAs comprise an annealed RNA:DNA duplex, wherein the sense strand of the duplex is a DNA molecule and the antisense strand of the duplex is a RNA molecule.

Preferably, each single stranded nucleic acid molecule of the siRNA duplex is about 21 nucleotides to about 27 nucleotides in length. In preferred embodiments, duplexed siRNAs have a 2 or 3 nucleotide 3′ overhang on each strand of the duplex. In preferred embodiments, siRNAs have 5′-phosphate and 3′-hydroxyl groups.

According to the present invention, siRNAs may be introduced to a target cell as an annealed duplex siRNA, or as single stranded sense and anti-sense nucleic acid sequences that once within the target cell anneal to form the siRNA duplex. Alternatively, the sense and anti-sense strands of the siRNA may be encoded on an expression construct that is introduced to the target cell. Upon expression within the target cell, the transcribed sense and antisense strands may anneal to reconstitute the siRNA.

The shRNAs to be used in the methods of the present invention comprise a single stranded “loop” region connecting complementary inverted repeat sequences that anneal to form a double stranded “stem” region. Structural considerations for shRNA design are discussed, for example, in McManus et al., RNA 8:842-850 (2002). In certain embodiments the shRNA may be a portion of a larger RNA molecule, e.g., as part of a larger RNA that also contains U6 RNA sequences (Paul et al., Nature Biotech 20:505-508 (2002).

In preferred embodiments the loop of the shRNA is from about 0 to about 9 nucleotides in length. In preferred embodiments the double stranded stem of the shRNA is from about 19 to about 33 base pairs in length. In preferred embodiments, the 3′ end of the shRNA stem has a 3′ overhang. In particularly preferred embodiments, the 3′ overhang of the shRNA stem is from 1 to about 4 nucleotides in length. In preferred embodiments, shRNAs have 5′-phosphate and 3′-hydroxyl groups.

RNA molecules may be chemically synthesized, for example using appropriately protected ribonucleoside phosphoramidites and a conventional DNA/RNA synthesizer. Suppliers of RNA synthesis reagents include Proligo (Hamburg, Germany), Dharmacon Research (Lafayette, Colo., USA), Pierce Chemical (part of Perbio Science, Rockford, Ill., USA), Glen Research (Sterling, Va., USA), ChemGenes (Ashland, Mass., USA), and Cruachem (Glasgow, UK). For example, single-stranded gene-specific RNA oligomers may be synthesized using 2′-O-(tri-isopropyl)silyloxymethyl chemistry by Xeragon A G (Zurich, Switzerland). Alternatively, RNA oligomers may be synthesized using Expedite RNA phosphoramidites and thymidine phosphoramidite (Proligo). RNAs produced by such methodologies tend to be highly pure and to anneal efficiently to form siRNA duplexes or shRNA hairpin stem-loop structures.

Following chemical synthesis, single stranded RNA molecules are deprotected, annealed to form siRNAs or shRNAs, and purified (e.g., by gel electrophoresis or High Pressure Liquid Chromatography). For example, siRNAs may be generated by annealing sense and antisense single strand RNA (ssRNA) oligomers. Similarly, shRNAs may be generated by annealing of complementary sequences within a single ssRNA molecule to form a hairpin stem-loop structure. The integrity and the dsRNA character of the annealed RNAs may be confirmed by gel electrophoresis and quantified by spectroscopy (using the standard conversion, wherein 1 unit of Optical Density at 260 nm=40 μg of duplex RNA/ml).

Most conveniently, siRNAs may be obtained from commercial RNA oligomer synthesis suppliers, which sell RNA-synthesis products of different quality and cost. For example, commercial suppliers of siRNAs include Dharmacon, Xeragon Inc. (now a QIAGEN company), Proligo, and Ambion.

Standard procedures may used for in vitro transcription of RNA from DNA templates carrying RNA polymerase promoter sequences (e.g., T7 or SP6 RNA polymerase promoter sequences). Efficient in vitro protocols for preparation of siRNAs using T7 RNA polymerase have been described (Donzé and Picard, Nucleic Acids Res. 30:46 (2002); and Yu et al., Proc. Natl. Acad. Sci. USA 99:6047 (2002)). Similarly, an efficient in vitro protocol for preparation of shRNAs using T7 RNA polymerase has been described (Yu et al., supra).

For example, sense and antisense RNA oligonucleotides for siRNA preparation may be transcribed from a single DNA template that contains a T7 promoter in the sense direction and an SP6 promoter in the antisense direction. Alternatively, sense and antisense RNAs may be transcribed from two different DNA templates containing a single T7 or SP6 promoter sequence. The sense and antisense transcripts may be synthesized in two independent reactions or simultaneously in a single reaction. Similarly, a ssRNA may be synthesized from a DNA template encoding a shRNA. The transcribed ssRNA oligomers are then annealed and purified. siRNAs may be generated by annealing sense and antisense ssRNA oligomers. Similarly, shRNAs may be generated by annealing of complementary sequences within a single ssRNA molecule to form a hairpin stem-loop structure. The integrity and the dsRNA character of the annealed RNAs may be confirmed by gel electrophoresis and quantified by spectroscopy (using the standard conversion, wherein 1 unit of Optical Density at 260 nm=40 μg of duplex RNA/ml).

RNAi probes may be formed within a cell by transcription of RNA from an expression construct introduced into the cell. For example, a protocol and expression construct for in vivo expression of siRNAs is described in Yu et al., supra. Similarly, protocols and expression constructs for in vivo expression of shRNAs have been described (Brummelkamp et al., Science 296:550 (2002); Sui et al., Proc. Natl. Acad. Sci USA. 99:5515 (2002); Yu et al., supra; McManus et al., RNA 8:842 (2002); and Paul et al., Nature Biotech. 20:505 (2002)).

For example, an siRNA may be reconstituted in a cell by use of an siRNA expression construct that upon transcription within the cell produces the sense and antisense strands of the siRNA. These complementary sense and antisense RNAs then anneal to reconstitute the siRNA within the cell. In one embodiment, the sense and antisense strands are encoded by a single sequence of the expression vector flanked by two promoters of opposite transcriptional orientation, thereby driving transcription of the alternate strands of the sequence. In another embodiment, the sense and antisense strands are encoded by independent sequences within a single expression vector, where each independent sequence is operably linked to a promoter to drive transcription. In yet another embodiment, the sense and antisense strands are encoded by independent sequences on two independent expression constructs, where each independent sequence is operably linked to a promoter to drive transcription.

Similarly, shRNAs may be generated in vivo by transcription of a single stranded RNA from an expression construct within a cell. The complementary sequences of the inverted repeat within the ssRNA then anneal to yield the stem-loop structure of the shRNA.

Expression construct-encoded RNAi probes have distinct advantages over their chemically synthesized or in vitro transcribed counterparts. They are cost effective and provide a stable and continuous expression of RNAi probe that is useful for analysis of phenotypes that develop over extended periods of time.

The expression constructs for in vivo production of RNAi probes comprise RNAi probe encoding sequences operably linked to elements necessary for the proper transcription of the RNAi probe encoding sequence(s), including promoter elements and transcription termination signals. Preferred promoters for use in such expression constructs include the polymerase-III HI-RNA promoter (see, e.g., Brummelkamp et al., supra) and the U6 polymerase-III promoter (see, e.g., Sui et al., supra; Paul et al. supra; and Yu et al., supra).

The RNAi probe expression constructs may further comprise vector sequences that facilitate the cloning and propagation of the expression constructs. Standard vectors useful in the current invention are well known in the art and include (but are not limited to) plasmids, cosmids, phage vectors, viral vectors, and yeast artificial chromosomes. The vector sequences may contain a replication origin for propagation in E. coli; the SV40 origin of replication; an ampicillin, neomycin, or puromycin resistance gene for selection in host cells; and/or genes (e.g., dihydrofolate reductase gene) that amplify the dominant selectable marker plus the gene of interest. Prolonged expression of the encoded RNAi probe in cell culture may be achieved by the use of vectors sequences that allow for autonomous replication of an extrachromosomal construct in mammalian host cells (e.g., EBNA-1 and oriP from the Epstein-Barr virus).

The RNA of RNAi probes may include one or more modifications, either to the phosphate-sugar backbone or to the nucleoside. For example, the phosphodiester linkages of natural RNA may be modified to include at least one heteroatom, such as nitrogen or sulfur. In this case, for example, the phosphodiester linkage may be replaced by a phosphothioester linkage. Similarly, bases may be modified to block the activity of adenosine deaminase. Where the RNAi candidate or probe is produced synthetically, or by in vitro transcription, a modified ribonucleoside may be introduced during synthesis or transcription. For example, incorporation of 2′-aminouridine, 2′-deoxythymidine, or 5′-iodouridine into the sense strand of an RNAi probe is tolerated by the RNAi pathway, whereas the same substitutions on the antisense strand of the RNAi is not (Parrish et al., Mol Cell. 6:1077 (2000)). Also, if a siRNA has a 2 or 3 nucleotide 3′ overhang on each strand of the duplex, substitution of 2′-deoxythymidine for uridine in the overhangs is tolerated by the RNAi pathway.

Effective strategies to deliver siRNAs to target cells in cell culture include physical or chemical transfection. An alternative strategy uses the endogenous expression of siRNAs by various RNA-polymerase III (Pol III) promoter expression cassettes that allow transcription of functional siRNAs or their precursors (Scherr et al., Curr. Med. Chem. 10(3):245-56 (2003)). Recently, a Pol III dependent promoter (H1-RNA promoter) was inserted in the lentiviral genome to drive the expression of a small hairpin RNA (shRNA) against enhanced green fluorescent protein (GFP-Abbas-Turki et al., Hum. Gene Ther. 13(18):2197-201 (2002)). siRNA can also be delivered in a viral vector derived, e.g., from a lentivirus (Tiscornia et al., Proc. Natl. Acad. Sci. U.S.A. 2003; 100:1844-8). For review articles, see Hannon, Nature 418:244-51 (2002) and Bernstein et al., RNA 7(11):1509-21 (2001). This technology also has been described in cultured mammalian neurons in Krickevsky and Kosik, Proc. Natl. Acad. Sci. USA 99(18): 11926-9 (2002). siRNA technology is also being used to make transgenic animals (Cornell et al., Nat. Struct. Biol. 10(2):91-2 (2003)). RNA is described in Publication Nos. WO 99/49029 and WO 01/70949.

Antisense Nucleic Acids. In a specific embodiment, to achieve inhibition of expression of an mBRAF gene, the nucleic acid molecules of the invention can be used to design antisense oligonucleotides. An antisense oligonucleotide is typically 18 to 25 bases in length (but can be as short as 13 bases in length) and is designed to bind to a selected oncogenically activated BRAF mRNA. This binding prevents expression of that specific oncogenically activated BRAF protein. The antisense oligonucleotides of the invention comprise at least 6 nucleotides and preferably comprise from 6 to about 50 nucleotides. In specific aspects, the antisense oligonucleotides comprise at least 10 nucleotides, at least 15 nucleotides, at least 25, at least 30, at least 100 nucleotides, or at least 200 nucleotides.

The antisense nucleic acid oligonucleotides of the invention comprise sequences complementary to the BRAF mRNA. For mBRAF T1796A, there is only one base difference between wt and mutant. However, 100% sequence complementarity is not required so long as formation of a stable duplex (for single stranded antisense oligonucleotides) or triplex (for double stranded antisense oligonucleotides) can be achieved. The ability to hybridize will depend on both the degree of complementarity and the length of the antisense oligonucleotides. Generally, the longer the antisense oligonucleotide, the more base mismatches with the corresponding mRNA can be tolerated. One skilled in the art can ascertain a tolerable degree of mismatch by use of standard procedures to determine the melting point of the hybridized complex to favor binding to mutant instead of wt BRAF (e.g., by creating a several additional mutations in the oligonucleotide). In cells that have wt but activated BRAF, the wt sequence sequence is targeted for mutation.

The antisense oligonucleotides can be DNA or RNA or chimeric mixtures, or derivatives or modified versions thereof, and can be single-stranded or double-stranded. The antisense oligonucleotides can be modified at the base moiety, sugar moiety, or phosphate backbone, or a combination thereof. For example, a mBRAF -specific antisense oligonucleotide can comprise at least one modified base moiety selected from a group including but not limited to 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5-methoxycarboxymethyluracil, 5-methoxyuracil, 2-methylthio-N6-isopentenyladenine, uracil-5-oxyacetic acid (v), pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine.

In another embodiment, the oncogenically activated BRAF-specific antisense oligonucleotide comprises at least one modified sugar moiety, e.g., a sugar moiety selected from arabinose, 2-fluoroarabinose, xylulose, and hexose.

In yet another embodiment, the oncogenically activated BRAF-specific antisense oligonucleotide comprises at least one modified phosphate backbone selected from a phosphorothioate, a phosphorodithioate, a phosphoramidothioate, a phosphoramidate, a phosphordiamidate, a methylphosphonate, an alkyl phosphotriester, and a formacetal or analog thereof.

The antisense oligonucleotide can include other appending groups such as peptides, or agents facilitating transport across the cell membrane (see, e.g., Letsinger et al., Proc. Natl. Acad. Sci. USA 86: 6553-6556 (1989); Lemaitre et al., Proc. Natl. Acad. Sci. USA 84: 648-652 (1987); PCT Publication No. WO 88/09810) or blood-brain barrier (see, e.g., PCT Publication No. WO 89/10134), hybridization-triggered cleavage agents (see, e.g., Krol et al., BioTechniques. 6: 958-976 (1988)), intercalating agents (see, e.g., Zon, Pharm. Res. 5: 539-549 (1988)), etc.

In another embodiment, the antisense oligonucleotide can include α-anomeric oligonucleotides. An α-anomeric oligonucleotide forms specific double-stranded hybrids with complementary RNA in which, contrary to the usual β-units, the strands run parallel to each other (Gautier et al., Nucl. Acids Res. 15: 6625-6641 (1987)).

In yet another embodiment, the antisense oligonucleotide can be a morpholino antisense oligonucleotide (i.e., an oligonucleotide in which the bases are linked to 6-membered morpholine rings, which are connected to other morpholine-linked bases via non-ionic phosphorodiamidate intersubunit linkages). Morpholino oligonucleotides are resistant to nucleases and act by sterically blocking transcription of the target mRNA.

Similar to the above-described RNAi molecules, the antisense oligonucleotides of the invention can be synthesized by standard methods known in the art, e.g., by use of an automated synthesizer. Antisense nucleic acid oligonucleotides of the invention can also be produced intracellularly by transcription from an exogenous sequence. For example, a vector can be introduced such that it is taken up by a cell within which the vector or a portion thereof is transcribed to produce an antisense RNA. Such a vector can remain episomal or become chromosomally integrated, so long as it can be transcribed to produce the desired antisense RNA. Such vectors can be constructed by recombinant DNA technology methods standard in the art. Vectors can be plasmid, viral, or others known in the art, used for replication and expression in mammalian cells. In another embodiment, “naked” antisense nucleic acids can be delivered to adherent cells via “scrape delivery”, whereby the antisense oligonucleotide is added to a culture of adherent cells in a culture vessel, the cells are scraped from the walls of the culture vessel, and the scraped cells are transferred to another plate where they are allowed to re-adhere. Scraping the cells from the culture vessel walls serves to pull adhesion plaques from the cell membrane, generating small holes that allow the antisense oligonucleotides to enter the cytosol.

The present invention thus provides a method for inhibiting the expression of an activating BRAF gene in a eukaryotic, preferably mammalian, and more preferably rat, mouse dogs or human cell, comprising providing the cell with an effective amount of a BRAF-inhibiting antisense oligonucleotide.

Ribozyme Inhibition. In another embodiment, the expression of activating BRAF gene of the present invention can be inhibited by ribozymes designed based on the nucleotide sequence thereof. Ribozyme molecules catalytically cleave mRNA transcripts and can be used to prevent expression of the gene product. Ribozymes are enzymatic RNA molecules capable of catalyzing the sequence-specific cleavage of RNA (for a review, see Rossi, Current Biology 4: 469-471 (1994)). The mechanism of ribozyme action involves sequence-specific hybridization of the ribozyme molecule to complementary target RNA, followed by an endonucleolytic cleavage event. The composition of ribozyme molecules must include: (i) one or more sequences complementary to the target gene mRNA; and (ii) a catalytic sequence responsible for mRNA cleavage (see, e.g., U.S. Pat. No. 5,093,246).

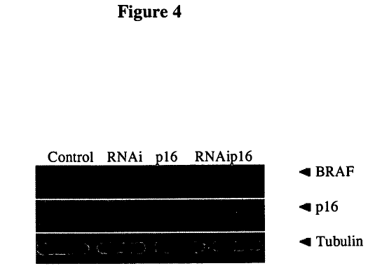

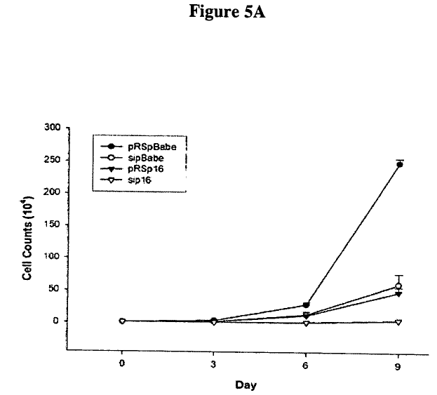

According to the present invention, the use of hammerhead ribozymes is preferred. Hammerhead ribozymes cleave mRNAs at locations dictated by flanking regions that form complementary base pairs with the target mRNA. The sole requirement is that the target mRNA has the following sequence of two bases: 5′-UG-3′. The construction of hammerhead ribozymes is known in the art, and described more fully in MYERS, MOLECULAR BIOLOGY AND BIOTECHNOLOGY: A COMPREHENSIVE DESK REFERENCE, VCH Publishers, New York (1995) (see especially FIG. 4, p. 833) and in Haseloff and Gerlach, Nature 334: 585-591 (1988).

Preferably, the ribozymes of the present invention are engineered so that the cleavage recognition site is located near the 5′ end of the corresponding mRNA, i.e., to increase efficiency and minimize the intracellular accumulation of non-functional mRNA transcripts.

As in the case of RNAi and antisense oligonucleotides, ribozymes of the invention can be composed of modified oligonucleotides (e.g., for improved stability, targeting, etc.). These can be delivered to mammalian cells, and preferably mouse, rat, dog or human cells, which express the target oncogenically activated BRAF protein in vivo. A preferred method of delivery involves using a DNA construct “encoding” the ribozyme under the control of a strong constitutive pol III or pol II promoter, so that transfected cells will produce sufficient quantities of the ribozyme to destroy endogenous mRNA encoding the protein and inhibit translation. Because ribozymes, unlike antisense molecules, are catalytic, a lower intracellular concentration may be required to achieve an adequate level of efficacy.

Ribozymes can be prepared by any method known in the art for the synthesis of DNA and RNA molecules, as discussed above. Ribozyme technology is described further in INTRACELLULAR RIBOZYME APPLICATIONS: PRINCIPALS AND PROTOCOLS (Rossi and Couture eds., Horizon Scientific Press) (1999).

Triple Helix Formation. Nucleic acid molecules useful to inhibit oncogenically activated BRAF gene expression via triple helix formation are preferably composed of deoxynucleotides. The base composition of these oligonucleotides is typically designed to promote triple helix formation via Hoogsteen base pairing rules, which generally require sizeable stretches of either purines or pyrimidines to be present on one strand of a duplex. Nucleotide sequences may be pyrimidine-based, resulting in TAT and CGC triplets across the three associated strands of the resulting triple helix. The pyrimidine-rich molecules provide base complementarity to a purine-rich region of a single strand of the duplex in a parallel orientation to that strand. In addition, nucleic acid molecules may be chosen that are purine-rich, e.g., those containing a stretch of G residues. These molecules will form a triple helix with a DNA duplex that is rich in GC pairs, in which the majority of the purine residues are located on a single strand of the targeted duplex, resulting in GGC triplets across the three strands in the triplex.

Alternatively, sequences can be targeted for triple helix formation by creating a so-called “switchback” nucleic acid molecule. Switchback molecules are synthesized in an alternating 5′-3′, 3′-5′ manner, such that they base pair with first one strand of a duplex and then the other, eliminating the necessity for a sizeable stretch of either purines or pyrimidines to be present on one strand of a duplex.

### p16^{INK4A} Restoration

p16^{INK4A} is inactivated in melanoma cells by several different ways including deletion, mutation, and promoter hypermethylation. p16^{INK4A} lesions are recessive loss of function defects. Different causes of loss of p16^{INK4A} activity may need to be corrected differently.

In instances where p16^{INK4A} is deleted, the present invention contemplates restoring the wt gene or protein, or achieving CDK4 or CDK6 inhibition by other means, such as through the use of CDK antagonists which bind to the CDKs and mimic p16^{INK4A} activity.

In instances where p16^{INK4A} is mutated, the present invention contemplates correcting the mutations, especially where the mutation is a dominant negative mutation which prevents activity of functional p16^{INK4A}. However, since mutations in tumor suppressor genes may have some residual activities, the preferred method is to introduce functional p16^{INK4A} into the cell rather than correct the mutation. Achieving CDK4 or CDK6 inhibition by other means, such as through the use of CDK antagonists which bind to the CDKs and mimic p16^{INK4A} activity should also apply.

In instances where the INK4A promoter is silent due to promoter hypermethylation, de-methylation is contemplated. There are known agents under evaluation in clinical trials for this purpose. Introduce functional p16^{INK4A} into the cell or achieving CDK4 or CDK6 inhibition by other means, such as through the use of CDK antagonists which bind to the CDKs and mimic p16^{INK4A} activity should also apply.

Some methods as described above for targeted alteration of BRAF can be employed to (i) restore expression of wt INK4A; (i) delete all or a portion of the coding sequence or regulatory sequence of the mutated INK4A gene (if necessary); (ii) replace all or a portion of the coding sequence or regulatory sequence of the mutated p16^{INK4A} gene with a different nucleotide sequence, e.g., replace the portion containing the inactivating mutation with wt or otherwise functional p16^{INK4A} sequences; (iii) insert into the coding sequence or regulatory sequence of the mutated p16^{INK4A} gene one or more nucleotides, or an oligonucleotide molecule, or polynucleotide molecule, which can comprise a nucleotide sequence from the same species or from a heterologous source (if necessary); or (iv) carry out some combination of (i), (ii) and (iii). This can be achieved using, e.g., heterologous recombination, gene activation technologies or gene therapy. In one preferred embodiment, RNA constructs are used to achieve this, as described in the examples below.

In one embodiment, all or a portion of the coding sequence or regulatory sequence of the mutated INK4A gene is deleted from a cell (e.g., when the mutant is dominant negative and interferes with wt gene) and a nucleic acid sequence encoding a wt INK4A in inserted into the cell. In a preferred embodiment, the wt sequence is inserted into the genome of the cell and is replicated into daughter (tumor) cells upon cell division. Such sequences can be inserted via a host of known vectors, such as retroviruses described herein, and described in the definitions above.

In another embodiment, all or a portion of the coding sequence or regulatory sequence of the mutated INK4A gene is deleted from a cell and protein transduction is used to provide a wt or functional p16^{INK4A} in to the cell. Protein transduction is the process by which peptide or protein motifs cross the cellular plasma membrane (Wadia et al., Current Opinion in Biotechnology 13:52-6 (2002)). The direct application of functional peptides and proteins to cells has been used to probe signal transduction pathways, block transcription factors, and conduct detailed structure/function analyses of integrin and other receptors' cytoplasmic domains, among other research applications (Hawiger et al., Current Opinion in Chemical Biology 3:89-94 (1999)). For example, a functional p16^{INK4A} can be modified be covalently coupled to a protein transduction domain (PTD) such as the one from the HIV-TAT protein. PTD's are generally short peptides, about 10-16 residues in length. Structurally dissimilar, their only common feature appears to be the presence of numerous positively charged lysine and arginine residues (Schwarze et al., Trends in Pharmacological Sciences 21:45-8 (2000)). Vectors based on HIV-TAT are. commercially available from, e.g., Invitrogen (Carlsbad, Calif.), and Phogen (United Kingdom). In another embodiment, a non-covalent carrier can be used to deliver the functional p16^{INK4A}. Such carriers include the Chariot® reagent from Active Motif Inc. (Carlsbad, Calif.) and is comprised of a short synthetic signaling peptide, called Pep-1, which does not require covalent coupling to the protein or peptide. In addition, lipid carriers are being employed to deliver proteins in addition to nucleic acids. For example, Gene Therapy Based Systems, Inc. and Imgenex, Inc. (both of San Diego, Calif.) have developed a new lipid formulation that interacts rapidly and noncovalently with protein, creating a protective vehicle for delivery (BioPORTER® and Provectin™, respectively-Zelphati et al., Journal of Biological Chemistry 276:35103-10 (2001)).

### p16 Mimicry

The present invention also contemplates restoration of p16^{INK4A} activity by contacting the cell with CDK antagonists, such as small molecules, which mimic p16^{INK4A} function. This rationale is based on of the fact that the biological function of p16^{INK4A} is to inhibit CDK4/6. There are several small molecule CDK inhibitors that are currently being evaluated in clinical trials. One is CYC202 (Cyclacel), and another is Flavopiridol (Aventis). Five additional compounds have been identified in cells with p16^{INK4A} lesions using CDK inhibition assays and CKD4 binding assays (3-ATA, BTD, NSC 625987, NSC 645153, NSC 521164, and flavopiridol) Kubo et al., Clinical Cancer Research 5; 4279-4286 (1999)). Of the compounds, flavopiridol was the most potent inhibitor of CDK4 activity (CYC202). These compounds (CYC202) did not affect p16^{INK4A} binding to CDK4 in vitro, suggesting that the mechanism of CDK4 inhibition by these compounds is not by competing with p16 binding to CDK4. However, as defined above, compounds designed or identified mimic p16INK4 binding to N-terminal region of CDK4, or inhibiting CDK4/6 expression/activity by other means are also contemplated for use in the present invention.

Other mimicry of p16^{INK4A} function include, for example, inhibiting the constitutive activation of the CDK4/6-RB-E2Fsignaling pathway using specific inhibitors for CDK4/6 or E2F molecules or by using more general agents such as phosphorylation inhibitors.

### Treatment

The present invention contemplates a method of treating a subject in need of such treatment, i.e., a subject having a tumor with a non-functional p16^{INK4A} and an oncogenically activated BRAF.

The term “treatment” means to therapeutically intervene in the development or pathology of a disease in a subject showing a symptom of this disease, e.g., inhibition of tumor growth and/or regression of a tumor, or inhibition of metastasis.

### Formulations and Administration

In one embodiment, nucleic acid sequences are administered to a subject in need thereof, e.g., mBRAF RNAi or sequences encoding a functional p16^{INK4A}. The nucleic acids according to the invention may be administered by any known methods, including methods used for gene therapy that are available in the art. The identified and isolated gene can then be inserted into an appropriate cloning vector. Vectors suitable for gene therapy include viruses, such as adenoviruses, adeno-associated virus (AAV), vaccinia, herpesviruses, baculoviruses and retroviruses, parvovirus, lentivirus, bacteriophages, cosmids, plasmids, fungal vectors and other recombination vehicles typically used in the art which have been described for expression in a variety of eukaryotic and prokaryotic hosts, and may be used for gene therapy as well as for simple protein expression.

In a preferred embodiment, the vector is a viral vector. Viral vectors, especially adenoviral vectors can be complexed with a cationic amphiphile, such as a cationic lipid, polyL-lysine (PLL), and diethylaminoethyldextran (DELAE-dextran), which provide increased efficiency of viral infection of target cells (See, e.g., PCT/US97/21496 filed Nov. 20, 1997, incorporated herein by reference). Preferred viral vectors for use in the present invention include vectors derived from vaccinia, herpesvirus, AAV and retroviruses. In particular, herpesviruses, especially herpes simplex virus (HSV), such as those disclosed in U.S. Pat. No. 5,672,344, the disclosure of which is incorporated herein by reference, are particularly useful for delivery of a transgene to a neuronal cell. AAV vectors, such as those disclosed in U.S. Pat. Nos. 5,139,941, 5,252,479 and 5,753,500 and PCT publication WO 97/09441, the disclosures of which are incorporated herein, are also useful since these vectors integrate into host chromosomes, with a minimal need for repeat administration of vector. For a review of viral vectors in gene therapy, see Mah et al., Clin. Pharmacokinet. 41(12):901-11 (2002); Scott et al., Neuromuscul. Disord. 12 Suppl 1:S23-9 (2002). In addition, see U.S. Pat. No. 5,670,488.

The coding sequences of the gene to be delivered are operably linked to expression control sequences, e.g., a promoter that directs expression of the gene. As used herein, the phrase “operatively linked” refers to the functional relationship of a polynucleotide/gene with regulatory and effector sequences of nucleotides, such as promoters, enhancers, transcriptional and translational stop sites, and other signal sequences. For example, operative linkage of a nucleic acid to a promoter refers to the physical and functional relationship between the polynucleotide and the promoter such that transcription of DNA is initiated from the promoter by an RNA polymerase that specifically recognizes and binds to the promoter, and wherein the promoter directs the transcription of RNA from the polynucleotide.

In one embodiment, a vector is used in which the coding sequences and any other desired sequences are flanked by regions that promote homologous recombination at a desired site in the genome, thus providing for expression of the construct from a nucleic acid molecule that has integrated into the genome (Koller and Smithies, Proc. Natl. Acad. Sci. USA 86:8932 8935 (1989); Zijlstra et al., Nature 342:435 438 (1989)).

In a specific embodiment, the vector is directly administered in vivo, where it enters the cells of the organism and mediates expression of the construct. This can be accomplished by any of numerous methods known in the art and discussed above, e.g., by constructing it as part of an appropriate expression vector and administering it so that it becomes intracellular, e.g., by infection using a defective or attenuated retroviral or other viral vector (see, U.S. Pat. No. 4,980,286), or by direct injection of naked DNA, or by use of microparticle bombardment (e.g., a gene gun; Biolistic, Dupont); or coating with lipids or cell surface receptors or transfecting agents, encapsulation in biopolymers (e.g., poly-β-1-64-N-acetylglucosamine polysaccharide; see U.S. Pat. No. 5,635,493), encapsulation in liposomes, microparticles, or microcapsules; by administering it in linkage to a peptide or other ligand known to enter the nucleus; or by administering it in linkage to a ligand subject to receptor mediated endocytosis (see, e.g., Wu and Wu, J. Biol. Chem. 62:4429 4432 (1987)), etc. In another embodiment, a nucleic acid ligand complex can be formed in which the ligand comprises a fusogenic viral peptide to disrupt endosomes, allowing the nucleic acid to avoid lysosomal degradation, or cationic 12 mer peptides, e.g., derived from antennapedia, that can be used to transfer therapeutic DNA into cells (Mi et al., Mol. Therapy 2:339 47 (2000)). In yet another embodiment, the nucleic acid can be targeted in vivo for cell specific uptake and expression, by targeting a specific receptor (see, e.g., PCT Publication Nos. WO 92/06180, WO 92/22635, WO 92/20316 and WO 93/14188). Recently, a technique referred to as magnetofection has been used to deliver vectors to mammals. This technique associates the vectors with superparamagnetic nanoparticles for delivery under the influence of magnetic fields. This application reduces the delivery time and enhances vector efficacy (Scherer et al., Gene Therapy 9:102-9 (2002)).

In a specific embodiment, the nucleic acid can be administered using a lipid carrier. Lipid carriers can be associated with naked nucleic acids (e.g., plasmid DNA) to facilitate passage through cellular membranes. Cationic, anionic, or neutral lipids can be used for this purpose. However, cationic lipids are preferred because they have been shown to associate better with DNA which, generally, has a negative charge. Cationic lipids have also been shown to mediate intracellular delivery of plasmid DNA (Felgner and Ringold, Nature 337:387 (1989)). Intravenous injection of cationic lipid-plasmid complexes into mice has been shown to result in expression of the DNA in lung (Brigham et al., Am. J. Med. Sci. 298:278 (1989). See also, Osaka et al., J. Pharm. Sci. 85(6):612-618 (1996); San et al., Human Gene Therapy 4:781-788 (1993); Senior et al., Biochemica et Biophysica Acta 1070:173-179 (1991); Kabanov and Kabanov, Bioconjugate Chem. 6:7-20 (1995); Liu et al., Pharmaceut. Res. (1996); 13; Remy et al., Bioconjugate Chem. 5:647-654 (1994); Behr, J-P., Bioconjugate Chem. 5:382-389 (1994); Wyman et al., Biochem. 36:3008-3017 (1997); U.S. Pat. No. 5,939,401 to Marshall et al; U.S. Pat. No. 6,331,524 to Scheule et al.

Representative cationic lipids include those disclosed, for example, in U.S. Pat. No. 5,283,185; and e.g., U.S. Pat. No. 5,767,099, the disclosures of which are incorporated herein by reference. In a preferred embodiment, the cationic lipid is N4-spermine cholesteryl carbamate (GL-67) disclosed in U.S. Pat. No. 5,767,099. Additional preferred lipids include N4-spermidine cholestryl carbamate (GL-53) and 1-(N4-spermine)-2,3-dilaurylglycerol carbamate (GL-89).

Preferably, for in vivo administration of viral vectors, an appropriate immunosuppressive treatment is employed in conjunction with the viral vector, e.g., adenovirus vector, to avoid immuno-deactivation of the viral vector and transfected cells. For example, immunosuppressive cytokines, such as interleukin-12 (IL-12), interferon-γ (IFN-γ), or anti-CD4 antibody, can be administered to block humoral or cellular immune responses to the viral vectors. In that regard, it is advantageous to employ a viral vector that is engineered to express a minimal number of antigens.

In embodiments where a non-nucleic BRAF antagonist or functional equivalent of p16^{INK4A} is administered (e.g., a small molecule), the therapeutic agent is administered in a pharmaceutical composition with a pharmaceutically acceptable carrier. The composition can be introduced parenterally, transmucosally, e.g., orally (per os), nasally, rectally, or transdermally, or by any well known to one of ordinary skill in the art. Parental routes include intravenous, intra-arteriole, intramuscular, intradermal, subcutaneous, intraperitoneal, intraventricular, and intracranial administration.

The phrase “pharmaceutically acceptable” refers to molecular entities and compositions that are physiologically tolerable and do not typically produce an allergic or similar untoward reaction, such as gastric upset, dizziness and the like, when administered to a human. Preferably, as used herein, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant, excipient, or vehicle with which the substance is administered. Such pharmaceutical carriers can be sterile liquids, such as water and oils, including those of petroleum, animal, vegetable or synthetic origin, such as peanut oil, soybean oil, mineral oil, sesame oil and the like. Water or aqueous solution saline solutions and aqueous dextrose and glycerol solutions are preferably employed as carriers, particularly for injectable solutions. Suitable pharmaceutical carriers are described in “Remington's Pharmaceutical Sciences” by E. W. Martin.

For oral administration, e.g., for small molecules, the pharmaceutical compositions may take the form of tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate.

Preparations for oral administration may be suitably formulated to give controlled release of the active compound. For buccal administration the compositions may take the form of tablets or lozenges formulated in conventional manner. For administration by inhalation, the chaperones for use according to the present invention are conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g., gelatin for use in an inhaler or insufflator may be formulated containing a powder mix of the compound and a suitable powder base such as lactose or starch.

The therapeutic agent may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The therapeutic agent may also be formulated in rectal compositions such as suppositories or retention enemas, e.g., containing conventional suppository bases such as cocoa butter or other glycerides.

In addition to the formulations described previously, the therapeutic agent may also be formulated as a depot preparation. Such long acting formulations may be administered by implantation (for example subcutaneously or intramuscularly) or by intramuscular injection. Thus, for example, the compounds may be formulated with suitable polymeric or hydrophobic materials (for example as an emulsion in an acceptable oil) or ion exchange resins, or as sparingly soluble derivatives, for example, as a sparingly soluble salt.

### Combination Therapy

Still another embodiment of the treatment comprises administering an anti-cancer therapy with the methods of the present invention. The anti-cancer therapy can be a chemotherapeutic agent, a nucleic acid encoding a cancer therapeutic agent, radiation therapy, a cancer vaccine, or an agent that affect the vasculature of a tumor. The cancer can be in the form of an operable tumor. The chemotherapeutic agent can be selected from the group consisting of cisplatin, 5-FU, mitomycin, etoposide, camptothecin, actinomycin-D, doxorubicin, verapamil, podophyllotoxin, daunorubicin, vincristine, vinblastine, melphalan cyclophosphamide, TNF-α, taxol and bleomycin.

In the combination therapy with an agent that affects tumor vasculature, the agent can be an anti-angiogenic agent selected from the group consisting of marimastat, COL-3, neovastat, thalidomide, squalamine, endostatin, angiostatin interferon-y, anti-VEGF antibody, or interleukin 12 (IL-12).

In the combination therapy with radiation therapy, the radiation therapy may be, but is not limited to, one or more of administering X-irradiation, UV-radiation, gamma-radiation, or microwave radiation. In a specific embodiment, the total dose of radiation is about 1 Gy to about 80 Gy. In another specific embodiment, the total does of x-ray radiation is between 200 and 6000 roentgens

### Dosages

The effective amount of therapeutic agent to be administered will depend, in part, on the agent, method of delivery, and, where applicable, specific amount and typical expression level of the therapeutic nucleic acid administered. The specific effective amount can be determined on a case-by-case basis, depending on the protein and corresponding therapeutic agent, by those skilled in the art.

Other factors to consider in determining doses are the individual's age, weight, sex, and clinical status. Pharmacokinetic and pharmacodynamics such as half-life (t_{1/2}), peak plasma concentration (cₘₐₓ) time to peak plasma concentration (tₘₐₓ), exposure as measured by area under the curve (AUC) and tissue distribution for the therapeutic agent can be obtained using ordinary methods known in the art to determine compatible amounts required in a dosage form to confer a therapeutic effect.

Data obtained from cell culture assay or animal studies may be used to formulate a range of dosages for use in humans. The dosage of compounds used in therapeutic methods of the present invention preferably lie within a range of circulating concentrations that includes the ED₅₀ concentration (effective for 50% of the tested population) but with little or no toxicity (e.g., below the LD5O concentration). The particular dosage used in any application may vary within this range, depending upon factors such as the particular dosage form employed, the route of administration utilized, the conditions of the individual (e.g., patient), and so forth.

A therapeutically effective dose may be initially estimated from cell culture assays and formulated in animal models to achieve a circulating concentration range that includes the IC₅₀. The IC₅₀ concentration of a compound is the concentration that achieves a half-maximal inhibition of symptoms (e.g., as determined from the cell culture assays). Appropriate dosages for use in a particular individual, for example in human patients, may then be more accurately determined using such information.

Toxicity and therapeutic efficacy of the composition can be determined by standard pharmaceutical procedures, for example in cell culture assays or using experimental animals to determine the LD₅₀ and the ED₅₀. The parameters LD₅₀ and ED₅₀ are well known in the art, and refer to the doses of a compound that is lethal to 50% of a population and therapeutically effective in 50% of a population, respectively. The dose ratio between toxic and therapeutic effects is referred to as the therapeutic index and may be expressed as the ratio: LD₅₀/ED₅₀. Chaperone compounds that exhibit large therapeutic indices are preferred.

The concentrations of the therapeutic agent will be determined according to the amount required to inhibit oncogenically activated BRAF or restore p16^{INK4A} activity, in the tumor.

### Screening Methods

The present invention further provides a method for screening for lead compounds to inhibit the BRAF or CDK gene products or nucleic acids. The methods generally involve contacting an BRAF or CKK protein- and nucleic acid-expressing cell with a test compound, determining the expression level or activity of an BRAF or CDK nucleic acid or protein in the cell, and comparing it to a control BRAF- or CDK-expressing cell not contacted by the compound.

The test compound can be, without limitation, a small organic or inorganic molecule, a polypeptide (including an antibody, antibody fragment, or other immunospecific molecule), an oligonucleotide molecule, a polynucleotide molecule, or a chimera or derivative thereof. Test compounds that specifically bind to BRAF-encoding nucleic acid molecule or to an BRAF protein of the present invention can be identified, for example, by high-throughput screening (HTS) assays, including cell-based and cell-free assays, directed against individual protein targets. Several methods of automated assays that have been developed in recent years enable the screening of tens of thousands of compounds in a short period of time (see, e.g., U.S. Pat. Nos. 5,585,277, 5,679,582, and 6,020,141). Such HTS methods are particularly preferred.

The expression level of the nucleic acid molecule in each of the treated and control cells can be determined by quantifying and comparing the amount of BRAF-encoding mRNA present in each of the first and second cells. Alternatively, the expression level of the nucleic acid molecule in each of the first and second cells can be determined by quantifying and comparing the amount of BRAF protein present in the first and second cells. Where the first cell has a detectable change in the expression level of the nucleic acid encoding an BRAF protein (e.g, BRAF mRNA) compared to the expression level of the nucleic acid encoding the BRAF protein (e.g., BRAF mRNA) in the second cell, the test compound is identified as a candidate compound useful for modulating the expression of an BRAF-encoding nucleic acid.

The present invention also provides a method for identifying a candidate compound that modulates an BRAF polypeptide. In one embodiment, the present invention provides a method for identifying a ligand or other binding partner to the BRAF protein, which comprises bringing a labeled test compound in contact with the BRAF protein or a fragment thereof and measuring the amount of the labeled test compound bound to the BRAF protein or to the fragment thereof. Whether the binding leads to inhibition of BRAF expression or activity will then need to be examined as described above.

Additionally, any of a variety of known methods for detecting protein-protein interactions may also be used to detect and/or identify proteins that bind to an BRAF gene product. For example, co-immunoprecipitation, chemical cross-linking and yeast two-hybrid systems as well as other techniques known in the art may be employed. As an example in a yeast two-hybrid assay, a host cell harbors a construct that expresses an BRAF protein or fragment thereof fused to a DNA binding domain and another construct that expresses a potential binding-partner fused to an activation domain. The host cell also includes a reporter gene that is expressed in response to binding of the BRAF protein-partner complex (formed as a result of binding of binding-partner to the BRAF protein) to an expression control sequence operatively associated with the reporter gene. Reporter genes for use in the yeast two-hybrid assay of the invention encode detectable proteins, including, but by no means limited to, chloramphenicol transferase (CAT), β-galactosidase (β-gal), luciferase, green fluorescent protein (GFP), alkaline phosphatase, and other genes that can be detected, e.g., immunologically (by antibody assay). See the Mammalian MATCHMAKER Two-Hybrid Assay Kit User Manual from Clontech (Palo Alto, Calif.) for further details on mammalian two-hybrid methods.

For detection of molecules using screening assays, a molecule (e.g., an antibody or polynucleotide probe) can be detectably labeled with an atom (e.g., radionuclide), detectable molecule (e.g., fluorescein), or complex that, due to its physical or chemical property, serves to indicate the presence of the molecule. A molecule can also be detectably labeled when it is covalently bound to a “reporter” molecule (e.g., a biomolecule such as an enzyme) that acts on a substrate to produce a detectable product. Detectable labels suitable for use in the present invention include any composition detectable by spectroscopic, photochemical, biochemical, immunochemical, electrical, optical or chemical means. Labels useful in the present invention include, but are not limited to, biotin for staining with labeled avidin or streptavidin conjugate, magnetic beads (e.g., Dynabeads™), fluorescent dyes (e.g., fluorescein, fluorescein-isothiocyanate (FITC), Texas red, rhodamine, green fluorescent protein, enhanced green fluorescent protein, lissamine, phycoerythrin, Cy2, Cy3, Cy3.5, Cy5, Cy5.5, Cy7, FluorX [Amersham], SyBR Green I & II [Molecular Probes], and the like), radiolabels (e.g., 3H, 125I, 35S, 14C, or 32P), enzymes (e.g., hydrolases, particularly phosphatases such as alkaline phosphatase, esterases and glycosidases, or oxidoreductases, particularly peroxidases such as horse radish peroxidase, and the like), substrates, cofactors, inhibitors, chemiluminescent groups, chromogenic agents, and colorimetric labels such as colloidal gold or colored glass or plastic (e.g., polystyrene, polypropylene, latex, etc.) beads. Examples of patents describing the use of such labels include U.S. Pat. Nos. 3,817,837; 3,850,752; 3,939,350; 3,996,345; 4,277,437; 4,275,149; and 4,366,241.

Means of detecting such labels are known to those of skill in the art. For example, radiolabels and chemiluminescent labels can be detected using photographic film or scintillation counters; fluorescent markers can be detected using a photo-detector to detect emitted light (e.g., as in fluorescence-activated cell sorting); and enzymatic labels can be detected by providing the enzyme with a substrate and detecting, e.g., a colored reaction product produced by the action of the enzyme on the substrate.

The screening methods described above can be modified for use in high-throughput screening, e.g., using microarrays.

### EXAMPLES

The present invention is further described by means of the examples, presented below. The use of such examples is illustrative only and in no way limits the scope and meaning of the invention or of any exemplified term. Likewise, the invention is not limited to any particular preferred embodiments described herein. Indeed, many modifications and variations of the invention will be apparent to those skilled in the art upon reading this specification and can be made without departing from its spirit and scope. The invention is therefore to be limited only by the terms of the appended claims along with the full scope of equivalents to which the claims are entitled.

A highly specific RNA interference approach was developed to inhibit the expression of the T1796A hot-spot BRAF mutation and to restore the expression of p16^{INK4A} in several melanoma cell lines that harbor both BRAF and INK4A defects.

### Example 1

### Inhibition of Oncogenic BRAF by RNAi

Rationale. 28 melanoma cell lines were screened for BRAF exon 15 mutations (Dong et al., Cancer Res. 2003; 63: 3883-3885). 22 cell lines were found to have a BRAF mutation (positive rate 79%). 20 of the mutations were at the hot spot T1796A transition. RNAi was then used to specifically inhibit the expression of the T1796A mutant BRAF (mBRAF). Specifically, a retroviral vector, pRETRO-SUPER-puro (pRS) (Brummelkamp et al., 2002, supra), which expresses the mBRAF RNAi as a short hairpin RNA (shRNA). The hairpin is cleaved by cellular factors to produce the mature RNAi (Agami, Curr Opin Chem Biol. 2002; 6:829-34.; Denli and Hannon, Trends Biochem Sci. 2003; 28:196-201).

The specificity and efficacy of the RNAi to knock down mutant BRAF expression was examined by (1) transient co-expression experiments in 293T cells, (2) comparison of changes of BRAF levels in BRAF wt and mutant LOH cells.

Results

Mutant BRAF RNAi reduced mBRAF to undetectable levels, but also somewhat inhibited expression of wt BRAF.

### Example 2

### Restoration of p16 or Suppression of Oncogenic BRAF Retards Melanoma Cell Growth

RNAi was used to specifically inhibit the expression of the T1796A mBRAF using a retroviral vector, and INK4A cDNA was used to restore wt INK4A expression in melanoma cells harboring both mBRAF and lost of expression of wt p16^{INK4A}. The data presented below is from combined studies.

### Methods

### BRAF

A retroviral vector, pRETRO-SUPER-puro (pRS-Brummelkamp et al., Cancer Cell. 2002(2):243-7) was used to generate control and mBRAF RNAi-expressing retroviruses, designated pRS-puro and pRS-puro mBRAF RNAi, respectively. The retroviruses were then used to infect a control melanoma cell line which expresses wt BRAF (Mel1363); an two melanoma cell lines that are heterozygous for mBRAF (624Mel and WM35), and one line exhibiting loss of heterozygosity and expressing only mBRAF (A375). Stable cell lines were generated after selection with puro and used in the subsequent assays. Cells were grown and lysed and then the cell lysates were separated by polyacrylamide gel electrphoresis (PAGE) and probed with anti-BRAF and anti-tubulin (control) antibodies. mBRAF RNAi reduced BRAF level in mBRAF positive cells, but not in BRAF wt melanoma cells. BRAF expression was most significantly reduced in A375 melanoma cells, which show LOH at the BRAF locus and thus express only mBRAF, which also demonstrate the specificity of the mBRAF RNAi.

Melanoma cells with mBRAF activating mutations have intrinsic ERK pathway activation, which is reflected by a high phospho-MEK signal under serum starved conditions.

In vitro assays. mBRAF RNAi expressing melanoma cells were also evaluated for in vitro growth in colony forming assays. 1×103 WM35 or 624Mel melanoma control cells, or those expressing mBRAF RNAi were plated in duplicate in 100 mm or 35 mm plates and grown in regular medium (with 10% or 5% serum for WM35 and 624Mel cells, respectively) for 3 weeks. Colonies were fixed and stained using known methods. Shown are representatives of three experiments.

To measure levels of wt and mBRAF inhibition by RNAi in 624Mel and WM35 melanoma cells (which contain both wt and mutant BRAF allele), a RT-PCR and restriction fragment length polymorphism (RFLP) assay was designed to semi-quantitate the expression of mutant and wt BRAF alleles. The PCR forward primer (5′-GTA AAA ATA GGT GAT TTT GGT CTA GCT GAA G-3′; SEQ ID NO: 5) is used to amplify BRAF exon 15 and to create, when the mutant A1796 follows in the DNA template, an MboII site [GAAGA(N8)] (SEQ ID NO: 6) and therefore a smaller sized fragment. The reverse primer is used had a sequence of 5′-CTC TTT TCT TTT TGA GGC ACT CTG C-3′ (SEQ ID NO: 7). PCR products are digested with MboII and restriction fragments separated by electrophoresis on a 3% agarose or 10% polyacrylamide gel. The method was validated by direct sequencing of PCR products. Using this method to compare levels of wt and mBRAF alleles in control and mBRAF RNAi expressing melanoma cells is ongoing. For quantitation, RT-PCR is done in the exponential phase of the PCR cycle (<30 cycles).

In vivo assays. In addition, to examine whether mBRAF inhibition could also affect tumorigenesis of melanoma cells in vivo, melanoma cells with and without mBRAF RNAi were xenografted into nude mice according to known methods. 5×105 BRAF mutation positive, 624Mel and WM35, and negative Mel1363 cells were injected subcutaneously into nude mice and monitored for tumor growth. The same amount of control cells were used as controls for the xenograft assays. Pictures were taken 8-10 weeks after cell inoculation. The experiments were repeated three times with the similar result.

Melanocyte differentiation assay. Melanoma cells expressing mutant BRAF RNAi exhibited increased pigmentation, produced more mature melanosomes and melanin, and expressed higher levels of tyrosinase and tyrosinase related protein-1 (TRP-1) suggesting that these cells were more differentiated. Melanogenesis was not induced by the restoration of wt INK4A although both expression of mBRAF RNAi and INK4A cDNA suppressed proliferation.

In order to investigate the molecular mediators of the color changes, the expression of melanocyte differentiation and pigmentation-related factor tyrosinase was studied. Tyrosinase is the rate-limiting enzyme in melanin production. Cell lysates of 624Mel control and cells expressing mBRAF RNAi were separated by PAGE gel. Western blot was probed with tyrosinase (TYR), TRP-1 and α-tubulin antibodies (control). The data are representative of three experiments.

pRB activity assay. To further define the mechanisms of the inhibitory effects of mBRAF suppression and p16^{INK4A} restoration, cell cycle retinoblastoma protein (pRB) phosphorylation was examined in control cells and those expressing mBRAF RNAi or p16^{INK4A}.

Cell death assays. TUNEL and FACS assays were used to assess cell death in response to transfection with the RNAi retroviral constructs.

### p16^{INK4A}

Screening for INK4A expression and mutation in melanoma cells. p16^{INK4A} expression was evaluated in seven melanoma cell lines (WM35, RPMI, 624Mel, Mel1363, A375, A101D, and OM431) using Western blotting. 293T control (293T), 293T transfected with INK4A cDNA (293T+p16) were used as controls. WM35, RPMI, 624Mel, Mel1363, A375, A101D, and OM431 melanoma cell lines were cultured in regular media. Cells were lysed, and the lysates were separated by PAGE and transferred onto nitrocellulose. The Western blot was probed with anti-p16^{INK4A} and anti-tubulin antibodies. WM35, RPMI, 624Mel, A375, A101D, and OM431 also have the BRAF T1796A mutation. p16^{INK4A} signals were detected using a highly sensitive SuperSignal West Pico Chemiluminecscent substrate (Pierce) that can detect pico-grams, instead of nano-grams (Amersham) of proteins. Since normal melanocytes from the matching melanoma cells were not available, the functional significance of the expressed p16^{INK4A} in those melanoma cell lines was examined instead by mutation analyses of INK4A.

Immunohistochemical analysis. Immunohistochemical analysis of p16^{INK4A} in formalin-fixed and paraffin-embedded melanocytic lesions including nevus, RGP, VGP, and metastatic melanomas was performed. Two commercial antibodies specific for p16^{INK4} were used-a mouse monoclonal from Oncogene (Ab-1, cat # NA29) and a polyclonal from Santa Cruz (c-20, cat# sc-468). For immunohistochemistry, tissue sections were treated to retrieve antigen and block background, and incubated with primary and secondary antibodies. Signals will be developed using streptavidin-HRP (Zymed Labs).

In vitro and in vivo assays. The wt INK4A cDNA was subcloned into the pBabe-neo retroviral vector. Melanoma cells were infected with either vector control or pBabe-neo-INK4A retroviruses. Cells were selected in G418, and the experiments described above for BRAF were performed for p16^{INK4A}. Briefly, for colony forming assays, 1×103 624Mel were plated in duplicate in 10 cm diameter plates and grown in regular medium for 14 days prior to fixing and staining. For xenograft assays, 1×106 wt p16^{INK4A} expressing or control 624Mel cells were injected subcutaneously into nude mice and monitored for tumor growth. Pictures were taken 8 weeks after cell inoculation

### Results

### BRAF

Reduction of mBRAF protein by RNAi inhibited endogenous phospho-MEK levels in melanoma cells that are mBRAF positive. BRAF was not significantly affected in Mel1363 cells that have wt (WT) BRAF, but was inhibited in 624Mel and WM35 cells that are heterozygous for mBRAF (HET). Mutant BRAF RNAi almost completely abolished BRAF expression in A375 cells that are mBRAF LOH and thus express only the mutant BRAF.

mBRAF RNAi significantly inhibited the growth of these cells in tissue culture in vitro as measured by cell counting and colony formation assay using regular media with serum. In vivo, BRAF mutation positive 624Mel and WM35, and negative Mel1363 cells were used for the experiments. Tumor growth was significantly inhibited by mBRAF RNAi in 624Mel and WM35 cells, but only slightly inhibited in Mel1363 cells.

Of note, melanoma cells expressing mBRAF RNAi not only grew slower, but also were darker in color and produced more mature melanosomes. Since melanosome maturation and melanin production are signatures of melanocyte differentiation, mBRAF RNAi expressing cells are therefore seem to be more differentiated. De-differentiation is characteristic of many tumors cells. In some cell types, it is caused by constitutive activation of the RAS/RAF/MEK/ERK signaling (Chang et al., Int J Oncol. 2003; 22:469-80; Englaro et al., J. Biol. Chem. 1988; 273:9966-70). Suppression of mBRAF causes inhibition of the ERK signaling, which may explain the observed phenotype. Tyrosinase, the rate-limiting enzyme in melanin production, was up-regulated in cells expressing mBRAF RNAi. The expression of tyrosinase related protein 1 (TRP-1) correlated with changes of tyrosinase in these cells.

Preliminary analyses using TUNEL and FACS showed that there was no significance difference in cell death between control and mBRAF RNAi expressing 624Mel, WM35, and Mel1363 cells (not shown).

### p16^{INK4A}

Among these cells, RPMI, 624Mel, and 1363Mel, have detectable levels of p16^{INK4A}. Mutation analysis of INK4A showed that 624Mel cells have an 18 bp in-frame deletion of codons 32-37 (CTGGAGGCGGGGGCGCTG-SEQ ID NO: 8) in exon la. The deleted sequence is located in the first ankyrin repeat and encodes an evolutionarily conserved six amino acids (LEAGAL) (Greenblatt, M. S. et al., Oncogene 22: 1150-1163 (2003)). Deletion and mutation affecting these amino acid have been reported in melanomas and other cancers and have been shown to significantly affect the CDK- and cell cycle-inhibitory activities of p16^{INK4A} (Harland, M., et al., Hum. Mol. Genet. 1997; 6: 2061-2067; Muzeau, F. et al., Int. J. Cancer. 1997; 72: 27-30; Ruas, M. and Peters, G. Biochim Biophys Acta. 1998; 1378: F115-177). The sequence shows LOH (not shown), suggesting that either the wt copy of the gene is deleted or that this is a homozygous deletion. Castellano et al., Cancer Res. 1997; 57:4868-4875 reported promoter hypermethylation in RPMI melanoma cells. These data suggest that p16^{INK4A} function is compromised in 624Mel and RPMI melanoma cells. Sequencing analysis showed that 1363Mel cells were wt for INK4A.

p16^{INK4A} was not detectable by Western blotting, nor was INK4A mRNA detected using RT-PCR at high cycle numbers (>40 cycles) with appropriate positive and negative controls in WM35 cells. PCR failed to amplify INK4A exons 1α and 2, although exon 3 is amplified and show normal sequence, suggesting that WM35 cells have homozygous deletion affecting INK4A exons 1α and 2.

p16^{INK4A} was highly expressed in nevus but lost or very low in melanoma cells, supporting the notion that loss of p16^{INK4A} is common in malignant melanomas. Melanoma cells expressing exogenous wt INK4A grew slower than control cells in vitro and in vivo, consistent with the tumor suppressor function of p16^{INK4A}. However, preliminary analyses showed that unlike cells expressing mBRAF RNAi, melanogenesis and the expression of tyrosinase and TRP-1 were not induced by INK4A.

Preliminary studies showed that the phosphorylation of serine 795 in pRB, a cyclin D1/CDK4 target, was reduced in 624Mel and WM35 melanoma cells expressing either mBRAF RNAi or p16^{INK4A}. These data are consistent with the involvement of pRB pathway proteins in mediating the growth inhibitory effects of mBRAF RNAi and p16^{INK4A}.

However, the observed suppression of melanogenesis, while unexpected would suggest that proliferation and differentiation are regulated differently by mBRAF and loss of INK4A in melanoma cells.

### Example 3A

### Effects on Melanoma Cells of Combined Inhibition of Oncogenic BRAF and Restoration of wt p16^{INK4A}

These experiments were performed to test the hypothesis that simultaneous inhibition of mBRAF and restoration of INK4A expression has a more potent effect against melanoma cells than inhibition of mBRAF or restoration of INK4A alone.

### Methods

Control cells and cells stably already expressing mBRAF RNAi were infected with control (pBabe-neo) or INK4 (pBabe-neo-INK4A) retroviruses as described above. G418 (geneticin plus puro) was used as a selection agent for transfection. The experiments were performed using 624Mel and WM35 melanoma cells, both of which harbor BRAF mutation and INK4A mutation/deletion. Mel1363 melanoma cells, which are BRAF wt and express low-level, wt INK4A, were used as control.

The mBRAF RNAi and INK4A co-expression experiment was then performed in the reverse order (infect INK4A expressing cells with mBRAF RNAi) (Table 1) and also in several other melanoma lines (including A375, RPMI, A101D, and OM431) to examine whether the effect is sequence and cell line dependent.

TABLE 1
Expression of mBRAF RNAi and INK4A in melanoma cells

| | | | |
|---|---|---|---|
| Set 1 | Established line | pRS-puro | pRS-puro mRAF |
| (RNAi | 2^{nd} control | pBabe-neo | RNAi |
| first) | 2^{nd} Infection | pBabe-neo-INK4A | pBabe-neo |
| | | | pBabe-neo-INK4A |
| Set 2 | Established line | pBabe-neo | pBabe-neo-INK4A |
| (INK4A | 2^{nd} control | pRS-puro | pRS-puro |
| first) | 2^{nd} Infection | pRS-puro mBRAF | pRS-puro mRAF |
| | | RNAi | RNAi |
| Set 3 | co-infection | pRS-puro | pBabe-neo |
| (together) | | pRS-puro mRAF | pBabe-neo-INK4A |
| | | RNAi | |

### Results

As summarized in Table 2 below, correction of both mBRAF and INK4A lesions in 624Mel and WM35 cells was lethal to the cells when mBRAF RNAi was expressed ahead of wt INK4A.

TABLE 2
The sequence of mBRAF RNAi and INK4A
expression affect cell survival

| Cell line | Set 1 | | Set 2 | |
|---|---|---|---|---|
| 624Mel | control | viable | control | Viable |
| | mBRAF RNAi + | lethal | INK4A + mBRAF | Viable |
| | INK4A | | RNAi | |
| WM35 | control | viable | control | Viable |
| | mBRAF RNAi + | lethal | INK4A + mBRAF | Viable |
| | INK4A | | RNAi | |
| 1363Mel | control | viable | control | Viable |
| | mBRAF RNAi + | viable | INK4A + mBRAF | Viable |
| | INK4A | | RNAi | |

These data demonstrate that correcting both lesions of BRAF and INK4A can generate lethal effects in melanoma cells. The results also suggest that BRAF mutation and INK4A loss-of-expression may synergize in melanoma pathobiology, which makes them ideal targets in melanoma treatment.

When INK4A-expressing cells were subsequently infected with mBRAF RNAi (Set 2, Table 2), viable cell lines were established. This suggests that the order of correcting BRAF and INK4A lesions affect the viability of the cells. Correcting mBRAF lesion first created a cellular envirenment that cannot tolerate wt INK4A in the cells in 624Mel and WM35 cells in our system. For example, it is hypothesized that inhibition of mBRAF causes melanogenesis and differentiation of melanoma cells. As a result, these differentiated cells might be more sensitive to the inhibitory effects of p16^{INK4A}. Thus, cell lines could not be established if mBRAF was inhibited first.

Therefore, the method of the present invention is achieved by either simultaneous inhibition of mBRAF and restoration of p16^{INK4A}, or by first inhibition mBRAF followed by restoration of p16^{INK4A}.

### Example 3B

### Combined Inhibition of BRAF by RNAi and Mimicry of p16^{INK4A} Using Small Molecule CDK Inhibitor Flavopiridol

In order to evaluate whether similar lethal effects on melanoma cells could be elicited by functionally mimicking the role of p16^{INK4A}, e.g., by inhibiting CDK4. To this end, a known CDK inhibitor, flavopiridol, was administered to melanoma cells in conjunction with mBRAF RNAi.

### Methods

Control 624Mel cells and 624Mel cells stably expressing mBRAF RNAi as described above in Example 2 were treated for 3 weeks with or without 10 nM flavopiridol (Aventis). Melanoma tumor growth was evaluated using a colony forming assay as described above in Example 2. Flavopiridol is a known CDK inhibitor which is being evaluated in clinical trials for treating several malignancies (Senderowicz A M, Invest New Drugs. 1999; 17:313-20). In a pilot study of 17 patients with untreated, advanced, melanoma, there was no response after treatment for a median of about 3 months (Burdette-Radoux et al., Invest New Drugs. 2004; 22: 315-322).

### Results

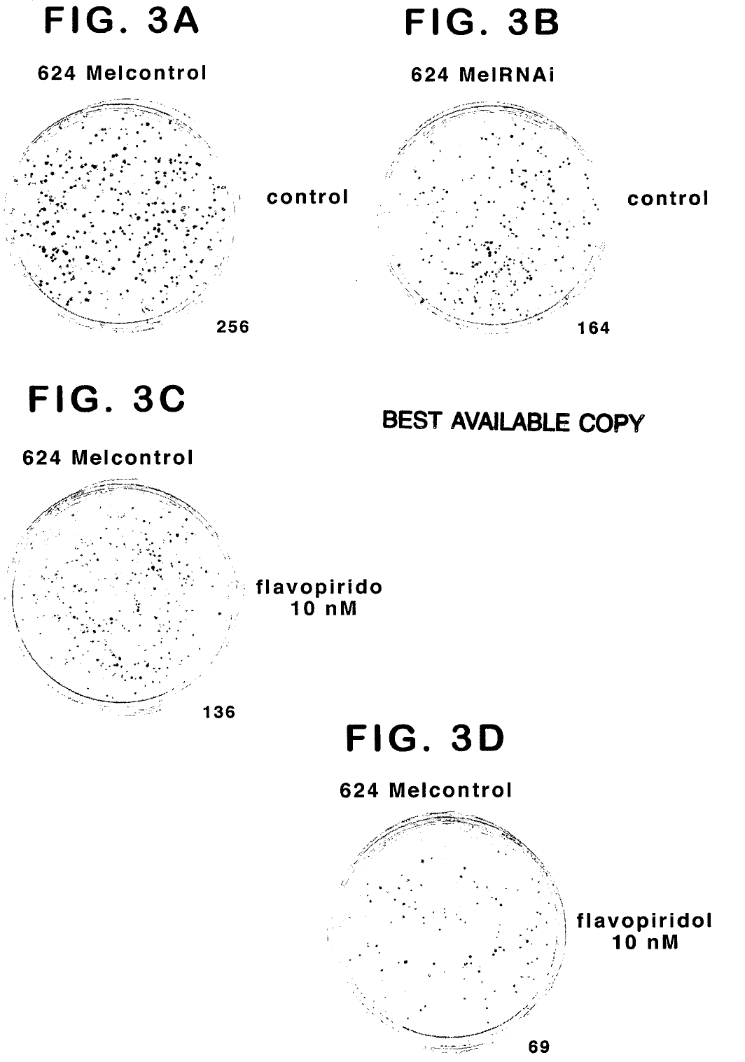

As shown in FIGS. 3A-D, the combination of 10 nM flavopiridol and mBRAF RNAi i Inhibited 624Mel cell growth to a greater extent than treatment with either mBRAF RNAi (3B) alone or flavopiridol alone (3C). This demonstrates that mimicking the p16^{INK4A} function, by inhibiting CDKs, including CDK4, can act additively or synergistically with BRAF suppression to inhibit melanoma cell growth.

### Example 4

### Simultaneous Inhibition of BRAF and INK4A Lesions Synergizes in Growth Suppression and Apoptosis in Melanoma Cells

These experiments were performed to test the hypothesis that simultaneous suppression of both BRAF and INK4A lesions promoted the growth inhibitory effect and synergized in the induction of apoptosis in melanoma cells.

Sequence-dependent viability of 624Mel melanoma cells expressing both mutant BRAF RNAi and wt p16. The pRS-puro and pBabe-neo retroviral vectors were produced and used to express the mBRAF RNAi and wt INK4A cDNA, respectively. Retroviruses were produced as described in Rotolo, S. et al., Effects on proliferation and melanogenesis by inhibition of mutant BRAF and expression of wt INK4A in melanoma cells, INT. J. CANCER (2005). For stable expression of both the RNAi and INK4A, melanoma cells stably expressing the BRAF RNAi and INK4A constructs were each respectively infected with a second retrovirus expressing the INK4A and BRAF RNAi constructs. A mass culture was selected and maintained in medium containing 1 μg/ml puromycin (RNAi) and 750 μg/ml G418 (INK4A).

Two sets of experiments were performed to express both mBRAF RNAi and wt INK4A in 624Mel and, as a control, in Mel1363 melanoma cells that express wt BRAF and p16^{INK4A} as described in Rotolo, S. et al., supra. As shown in Table 3, viable cells expressing both mBRAF RNAi and wt INK4A were obtained only in the Set 1 experiment for 624Mel cells. However, Mel1363 control cells were viable under the same conditions.

TABLE 3
Generation of 624Mel cells expressing
both mBRAF RNAi and wt INK4A

| | | | |
|---|---|---|---|
| Set 1 | Established | pRS-puro | pRS-puro mRAF RNAi |
| (RNAi | line | pBabe-neo (viable) | pBabe-neo (viable) |
| first) | 2^{nd} control | Babe-neo-INK4A | Babe-neo-INK4A |
| | 2^{nd} Infection | (viable) | (lethal) |
| Set 2 | Established | pBabe-neo | pBabe-neo-INK4A |
| (INK4A | line | pRS-puro (viable) | pRS-puro (viable) |
| first) | 2^{nd} control | PRS-puro BRAF | PRS-puro mRAF RNAi |
| | 2^{nd} Infection | RNAi (viable) | (viable) |

Levels of BRAF and p16 proteins. The efficiency of BRAF inhibition and p16 expression were examined by immunoblotting, the results of which are shown in FIG. 4. The Western blot was probed with BRAF, p16, and tubulin antibodies. Cell lysates were separated by PAGE and probed with BRAF, p16^{INK4A} and tubulin antibodies. The control, RNAi, p16, and RNAip16 are cells expressing pRS plus pBabe, mBRAF RNAi plus pBabe, pRS plus wt INK4A, and mBRAF RNAi plus wt INK4A, respectively.

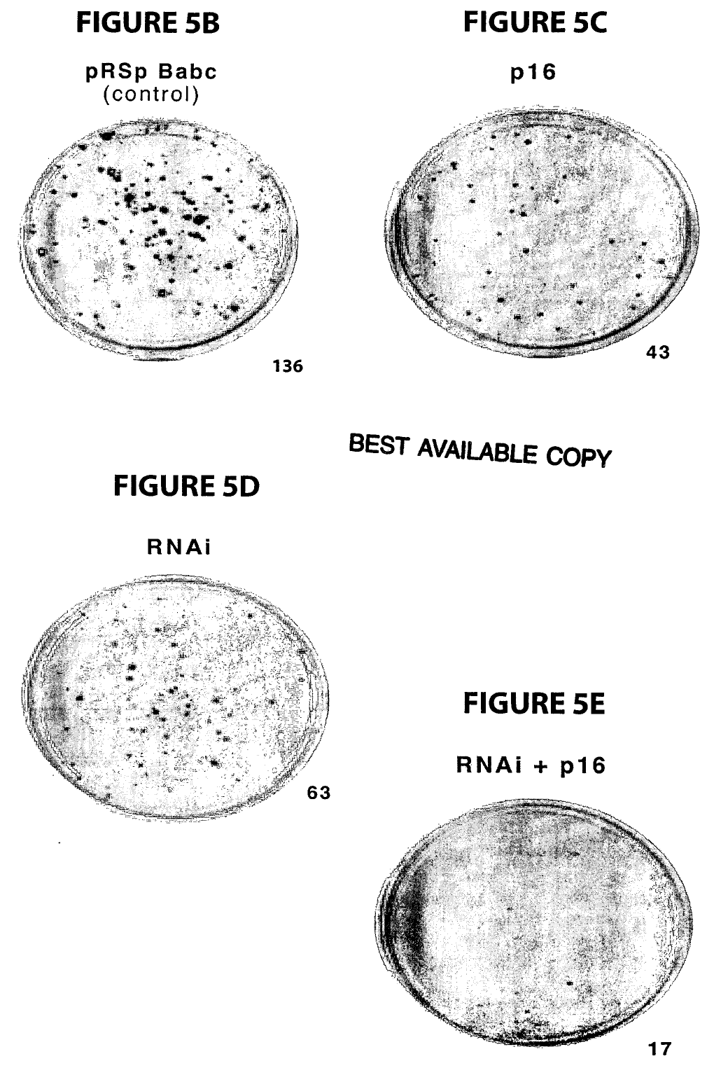

Enhanced growth inhibition by simultaneous suppression of mBRAF and expression of wt INK4A. Simultaneous expression of mBRAF RNAi and wt INK4A significantly inhibited the growth of 624Mel cells in tissue culture, as measured by cell count (FIG. 5A) and colony formation assay (FIG. 5B).

For cell counting (FIG. 5A), 1×10⁵ melanoma control cells, those stably expressing either mBRAF RNAi, INK4A, or both mBRAF RNAi and INK4A were plated in triplicate in 100 mm diameter plates and grown in DMEM supplemented with 5% FBS. Cells were counted at 3, 6 and 9 days after plating cells.

For colony formation assay (FIG. 5B), 1×10³ 624Mel melanoma control cells (pRSpBabe), cells expressing mBRAF RNAi plus pBabe (RNAi), cells expressing pRS plus INK4A (p16), or cells expressing both RNAi and INK4A (RNAip16) were plated in triplicate in 35 mm diameter plates and grown in medium with 5% serum for 3 weeks. Colonies were fixed, stained, and counted. The combination effect on growth inhibition was not limited to 624Mel cells because similar effect was observed in other melanoma lines that harbor both BRAF and INK4A lesions (cell lines OM431 and A375; not shown).

Both the cell count and colony numbers shown are average counts from 3 plates (p<0.001, ANOVA and p<0.001, two Poisson parameters test, respectively).

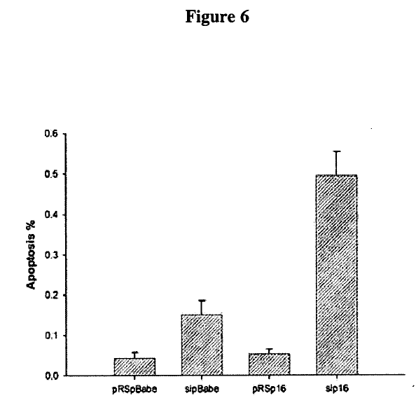

Inhibition of mBRAF and restoration of wt INK4A synergize in promoting apoptosis. Analysis of apoptosis by terminal deoxynucleotidyl transferase-mediated dUTP-biotin nick end labeling of DNA fragments (TUNEL) assay showed that expression of mBRAF RNAi and wt INK4A synergize in the induction of apoptosis (FIG. 6).

For apoptosis assay, 624Mel cells were cultured in four-well chambers in media with 5% serum until about 50-70% confluent, fixed in 4% paraformaldehyde in PBS for 1 hour at room temperature, and washed with PBS. Cells were permeabilized with 0.1% Triton X-100 and 0.1% sodium citrate for 2 minutes at 4° C. After rinsing, slides were incubated with 50 μL of TUNEL reaction mixture, containing TdT- and FITC-labeled dUTP (In Situ Cell Death Detection kit, Fluorescent, Roche Molecular Biochemicals) in a humidified atmosphere for 1 hour at 37° C. in the dark. Rinsed slides were then coverslipped with Vectashield mounting medium containing DAPI for nuclear counterstaining. TUNEL-positive cells which fluoresce bright green, were viewed with a Nikon Eclipse E1000 fluorescent microscope equipped with a FITC filter. Photomicrographs were taken with a Zeiss camera.

The percentage of apoptotic cells on total cell number was determined for each sample in a blind fashion by counting the number of green fluorescent nuclei among a total of 300 or more DAPI-stained blue nuclei in three randomly chosen fields. The dead cells detached from the plate and showed shrinkage and membrane blebbing, characteristic of apoptotic cells (Okada, H. et al., Pathways of apoptotic and non-apoptotic death in tumour cells, NAT. REV. CANCER, 4:592-603 (2004)). The percentage of apoptotic cells were presented (p<0.001, ANOVA).

### Example 5

### Studies of BRAF and INK4A Lesions on Proliferation, Differentiation, and Survival of Melanoma Cells

The above data suggest that although both mBRAF inhibition and INK4A reconstitution inhibit melanoma cell proliferation, they have different effects on melanogenesis and cellular differentiation. Therefore, the presence of mBRAF and loss of INK4A function have alternative roles in melanoma transformation. Separate regulation of proliferation and differentiation in melanoma cells by mBRAF and loss of INK4A will be an important point of investigation, since this phenomenon could have important implications in melanoma treatment when targeting mBRAF and INK4A defects, and since the underlying molecular mechanisms are basically unknown.

For future experiments, multiple in vitro growth parameters will be examined in additional melanoma cells. Further to the experiment described in Example 4, and in response to simultaneous mBRAF RNAi and restoration of wt INK4A, or, sequential mBRAF inhibition followed by restoration of wt INK4A, cell proliferation will be analyzed by cell doubling time. Cell cycle distribution will be studied by propidium iodide staining and FACS. Apoptosis will be further examined by FACS analyses of annexin V stained cells.

Metastasis and differentiation. Tumors that were inhibited in vivo by the simultaneous inhibition of mBRAF and restoration of wt INK4A will also be further evaluated. Specifically, whether these cells are reversibly or irreversibly growth arrested and terminally differentiated will be determined. Tumors will be removed and put back in culture to see how these cells grow compared with controls. Cells will be stained with SA-β-gal, a marker of growth arrest/senescence as described (Marcip et al., Embo J. 21:2180-8 (2002)). Dissected tumors will also be formalin-fixed and paraffin-embedded and studied for cell proliferation (PCNA and Ki-67 immunostaining) and apoptosis (TUNEL). It has been reported that the ERK pathway inhibitor CI 1040 suppressed pulmonary metastases of mBRAF positive melanoma cells in mice (Collisson et al., Cancer Res. 2003; 63:5669-73). Therefore, lung, liver, and brain, the three common sites of melanoma metastasis, will be examined in mice subcutaneously inoculated with control, mBRAF RNAi or INK4A expressing cells at the end of the xenograft experiments.

Alternatively, to best measure the roles of BRAF and INK4A lesions in melanoma metastasis, melanoma cells will be injected intravenously in SCID mice as described (Collisson et al., supra). The A375 line is well characterized with respect to metastasis in mice (Clark et al., Nature. 2000; 406:532-5) and will be used in these experiments. Control and A375 cells expressing mBRAF RNAi or INK4A, as well as cells simultaneous expressing mBRAF RNAi and restored INK4A, will be injected intravenously vial tail vein and monitored for metastasis in lung and other organs. Melan-A/MART-1, myelin-associated glycoprotein-1 (MAG-1), and glycoprotein 100 (gp100) are antigens specifically expressed in melanocytic lineage and clinically useful melanoma markers (Setaluri et al., J. Invest Dermatol. 2003; 121:650-60). Immunostaining with these antigens will be used to help identify metastatic melanoma cells.

Melanogenisis and differentiation. In addition, additional markers (other than tyrosinase) of melanocyte differentiation between control and melanoma cells expressing mBRAF RNAi and/or INK4A will be compared. Differentiation will be measured at the physiological level by melanin production and cell morphology using spectrophotometry and microscopy. There are four distinct stages of melanosome maturation that can be distinguished by morphology and melanin content (Setaluri et al., supra). Detailed examination of melanosomes by electron microscopy should identify to which stage cells containing mBRAF RNAi and exogenous INK4A have progressed. The electron microscopy experiments will be performed with help from Mount Sinai Microscopy Shared Research Facility.

It has been reported that the three melanogenic factors (tyrosinase, TRP-1, and DCT/TRP-2) responsible for melanin production are either under expressed or completely missing in melanoma cells (Eberle et al., Pigment Cell Res. 1995; 8:307-13; Hofbauer, J. Cutan. Pathol. 1998; 25:204-9). The levels and subcellular localization of tyrosinase, TRP-1, and DCT/TRP-2 will be further examined in control melanoma cells and those expressing mBRAF RNAi or INK4A using Western blotting, quantitative RT-PCR, and immunocytochemical assays. These experiments should help clarify whether BRAF and INK4A lesions have the same effects on these melanogenic factors, and whether the regulations are at the level of transcription, translation, or post-translation.

Cell proliferation and melanogenesis in mBRAF RNAi and/or INK4A-expressing and control cells will also be correlated. BrdU and tyrosinase will be used as markers of proliferation and melanogenesis, respectively, and monitored by double immunostaining (Dong et al., Embo. J. 1998; 17:2308-18). BrdU labeled cells will also be analyzed using immuno-electron microscopy and correlated with stages of melanosomes. These experiments should tell whether melanin formation correlates with S phase entry in melanoma cells expressing mBRAF RNAi or INK4A.

Preliminary data showed that although both mBRAF interference and p16^{INK4A} expression inhibited the growth of melanoma cells and caused decreased phosphorylation of serine 795 in pRB, melanogenesis was only increased by blocking mBRAF. Thus, while both BRAF and INK4A lesions affect cell proliferation, it appears that only mutant BRAF interferes with cellular differentiation.

### Expected Results

These studies should elucidate at which stage of melanosome maturation mBRAF has influence on melanogenesis, the dynamic relationship between BrdU incorporation and melanin production, and whether the three melanogenic factors (tyrosinase, TRP1, or DCT/TRP2) are regulated by mBRAF. Preliminary data indicates that tyrosinase, TRP1, and DCT/TRP2 mRNAs are up-regulated in mBRAF RNAi expressing cells. Accordingly, the expression of MITF, the transcriptional regulator of the three genes in control and mBRAF RNAi expressing melanoma cells will be examined.

### Example 6

### Evaluation of Cell Cycle in Melanoma Cells Harboring BRAF and INK4A Lesions

The molecular links between BRAF and INK4A lesions and proliferation and differentiation will be further dissected in melanoma cells. RB protein, microphthalamia-associated transcription factor (MITF), and D-type cyclins were reported to have dual roles in cell proliferation and differentiation (Setaluri, 2003, supra; Strasberg et al., Biochem Biophys Res Commun. 216:422-7 (1995)) and will be the focus of the initial investigation.

Several studies have demonstrated non-overlapping roles of pRB in proliferation and differentiation through interactions with different transcription factors (Gutierrez et al., Curr Opin Plant Biol. 5:480-6 (2002); Galderesi et al., Oncogene 22:5208-19 (2003)). Apart from its well-known interaction with E2F in the regulation of cell cycle progression, RB protein was shown to cooperate with differentiation-specific transcription factors, such as MyoD and C/EBP in cellular differentiation (Sellers et al., Genes Dev. 12:95-106 (1998); Ashizawa et al., J Biol. Chem. 276:11362-70 (2001)). Specific phosphorylation of pRB in several serine/threonine residules has been shown to regulate the interaction of RB with different transcription factors41. Whether similar interactions between pRB and differentiation proteins occur in melanoma cells, and whether the interactions are regulated by mBRAF and INK4A are unknown. The melanocyte-lineage specific master control protein MITF can potentially interact with pRB because it is a member of the basic helix-loop-helix family of transcription factors that have been shown to interact with pRB (e.g., MyoD). During normal development, differentiation stimuli trigger the activation of MITF to promote cell-cycle arrest and initiate the melanogenesis process (Setaluri, 2003, supra). It has been reported that the phosphorylation, degradation and transcription activities of MITF are regulated by ERK signaling (Kim et al., Int. J. Biochem Cell Biol. 36:1482-91 (2004); Widlund et al., Oncogene 22:3035-41 (2003). Since melanogenesis seems to be induced only by mBRAF inhibition, it is expected that MITF is affected only by mBRAF but not by INK4A restoration.

In addition, the three D-type cyclins (cyclin D1, D2, and D3) can be regulated by both mBRAF and p16^{INK4A}. Mitogen signals regulate the transcription, nuclear localization, and turnover of D-type cyclins (Ortega et al., Biochem. Biophys. Acta. 1602:73-87 (2002)). Cyclin D1 and D3 are over-expressed in melanomas and required for the growth and survival of melanoma cells in vitro and in vivo (Bartkova et al., Cancer Res. 6:5475-83 (1996); Florenes et al., Oncogene 13:2447-57 (1996)). Over-expression of cyclin D3 and suppression of cyclin D1 were reported to correlate with melanocyte differentiation (Strasberg et al., supra). Cyclin D1 was reported to be sequestered in the cytoplasm as neuronal cells underwent cell cycle withdrawal and terminal differentiation (Sumrejkanchanakij et al., Oncogene 22:8723-30 (2003); Radu et al., Mol. Cell. Biol. 23:6139-49 (2003)). Thus, it is expected that the expression and subcellular localization of D-type cyclins are regulated differently by lesions of BRAF and INK4A in melanoma cells.

The expression, phosphorylation, and subcellular localization of pRB will be examined in control and melanoma cells expressing mBRAF RNAi and/or INK4A by Western blotting and immunocytochemical assays. Western blotting may also detect slowly migrating phospho-pRB isoforms. Antibodies recognizing Ser780, Ser795, and Thr826 site-specific phosphorylated pRB (CDK4/CDK6 specific targets, Cell Signaling) will be used to detect pRB phosphorylation. The expression and subcellular localization of D-type cyclins and pocket proteins p107 and p130 between control and siRNA expressing melanoma cells will be examined by immunoblotting, RT-PCR, and immunocytochemical analyses. CDK4/6 kinase activity will be measured using in vitro kinase assay (Dong, 1998, supra) and correlated with changes in the status of pRB and cyclin D. Endogenous CDK4/6 proteins will be co-immunoprecipitated with cyclin D1 antibody and incubated with recombinant maltose binding protein-pRb fusion protein as a substrate (RB-C fusion protein, contains pRB residues 701-928, Cell Signaling) in the presence of [(-32P] ATP (NEN). Phosphorylated RB-C fusion protein will be resolved in polyacrylamide gel and levels of phosphorylation quantified by phosphoimage analysis

### Example 7

### Correlation of INK4A Expression and Oncogenic Mutation of BRAF in Melanoma Specimens

Expression of p16^{INK4A} in a panel of formalin-fixed and paraffin-embedded melanocytic lesions including nevi (benign), RGP (early melanoma), VGP (invasive melanoma), and metastatic melanomas will be examined. BRAF mutation status has been characterized in these samples (Dong et al., 2003, infra). The expression of p16^{INK4A} will be examined by immunohistochemical staining of tissue sections using commercially available antibodies (Oncogene (Ab-1, cat # NA29), Santa Cruz (c-20, cat# sc-468), NeoMarkers (Neo MS887),Pharmingen (G175-405). Since activating mBRAF may upregulate INK4A expression, unless p16^{INK4A} expression is blocked by genetic or epigenetic changes, all of the BRAF mutation positive samples may have high levels of p16^{INK4A}. The results will be tabulated on the basis of intensity and nuclear/cytoplasmic location of the signal.

Methods

Specimens with high levels of p16^{INK4A} expression will be screened for INK4A mutation. Pure populations of melanocytic lesions will be collected using laser capture microdissection (LCM). A population of cells that is >80% pure can be collected using LCM. Series sections are used to collect over 200 cells in each sample DNA will be extracted as described (Dong et al., 2003, supra). PCR conditions for mutation analysis of INK4A exons 1α, 2 and 3 were established previously Fargnoli M C, Chimenti S, Keller G, Soyer H P, Dal Pozzo V, Hofler H, et al. CDKN2a/p16^{INK4A} mutations and lack of p19ARF involvement in familial melanoma kindreds. J. Invest. Dermatol. 111(6): 1202-6 (1998).

PCR products will be purified using Qiagen PCR purification kit and sequenced using a Prism Model 3700 Capillary Array Sequencer and Big Dye Terminator Chemistry (Applied Biosystems). This procedure was successfully employed to identify INK4A mutation and deletion in 624Mel and WM35 melanoma cells. Clinical diagnostic laboratory quality control standards will be applied for the molecular analysis to avoid PCR contamination. INK4A status will be compared among various melanocytic lesions.

Statistical methods. A chi-square analysis based on the two-by-X contingency table, where the two rows represent p16^{INK4A} present or absent and the X columns represent the X categories of lesions, will be performed. In addition to testing the overall homogeneity of the X proportions, pairwise comparisons will be performed between the various categories. In situations where the proportions are very small, yielding a small expected number of events in at least one cell of the table, the chi square test may perform poorly. In this type of situation, Fisher's Exact Test will be used. PROC FREQ in the SAS statistical software package will be used, and it has the option of calculating significance based on either the chi-square test or Fisher's test. To protect against the increased risk of a Type I error in performing multiple tests, a Bonferroni correction will be made in assessing statistical significance. Appropriate statistical analysis will be performed to justify sample size.

Expected Results

Based on published studies and preliminary data, wt INK4A is expected to be highly expressed in the nevus cells (cytoplasm and nucleus), especially those harboring BRAF mutations. INK4A expression is expected to be absent, low, or mis-expressed (cytoplasm or nucleus only) in VGP and metastatic melanoma samples and may also be low or mis-expressed in RGP. In melanoma samples that have high level of p16^{INK4A}, an INK4A mutation may be identified. Nevus cells without INK4A expression while positive for BRAF mutation may express a “suppressor” of INK4A loss. For example, a component of the CDK4/6-cyclin D complex may be expressed at low level or be absent in such nevus cells. It has been shown that oncogenic RAS can decrease CDK4 expression in association with cell cycle arrest in G1 phase (Lazarov et al., Nat. Med. 8:1105-14 (2002)). Mutant BRAF may cause similar effect on CDK4 in these nevus cells, which can be examined by immunostaining of CDK4.

In contrast, if wt INK4A is highly expressed in VGP and metastatic melanoma cells harboring BRAF mutation, the cells may have high activities/mutations of CDK4/6 or the CDK4/6-cyclin D complexes may be resistant to inhibition by p16^{INK4A} as in the ES cells (Faast et al., Oncogene 23:491-502 (2004)). Levels of D-type cyclins and CDK4/6 in these samples can be analyzed by immunohistochemical analyses using commercially available antibodies. The CDK4 hot-spot R24C mutation can be analyzed (Wolfel et al., Science 269:1281-4 (1995); Zuo et al., Nat. Genet. 12:97-9 (1996)). Alternatively, loss of expression or mutation in RB family genes may be identified. Immunostaining of RB proteins and mutational analyses of RB DNAs can be performed. These analyses may be of clinical utility in the distinction between nevi and melanomas, if for example, INK4A is found to be expressed at high level in the great majority of nevi samples, but is low/absent or with mutations in melanomas.

### Example 8

### De Novo Transformation of Melanocyte Cells

An understanding of how activating BRAF and loss of INK4A cooperate in melanoma transformation can also be gained from knowledge of the biological and molecular consequences of their activity in normal human melanocytes. Thus, melanoma formation de novo will be recreated by activating BRAF and blocking INK4A expression. This will be achieved using BRAF wt and mBRAF (BRAF T1796A) expression retroviral constructs as described above in Example 2. We will design shRNA oligonucleotides targeting exon 1α of the INK4A/ARF locus to specifically inhibit the expression of INK4A. The INK4A shRNAi oligonucleotide will be subcloned into pRS-puro shRNA retroviral expression construct as for mBRAF RNAi.

Retroviruses will be generated and human melanocytes (commercially available) will be infected with INK4A shRNA or mutant BRAF virus separately, or combined. It is anticipated that it will necessary to express telomerase to bypass senescence in order to generate immortal cells in culture (Bennett and Medrano, Pigment Cell Res. 15:242-50 (2002)). The catalytic subunit of human telomerase reverse transcriptase (hTERT; Biroccio et al., Oncogene 21:3011-9 (2002)) can be expressed using a retroviral-hygromycin vector in control and in combination with INK4A shRNA or/and mBRAF. Cells stably expressing the retroviral constructs can be selected. In vitro and in vivo growth phenotypes of parental and viral infected cells can be analyzed and compared. Expression of mBRAF will cause growth arrest/senescence that can be measured by cell morphology and senescence-associated β-galactoside (SA-β-gal) expression (Macip et al., 2002, supra). Inhibition of INK4A expression will induce low grade malignant phenotypes mimicking RGP cells (e.g., unable to form tumor xenograft in nude mice), whereas mBRAF and INK4A RNAi together will transform hTERT-expressing melanocytes into malignant cells measured by colony-forming ability, and growth in soft agar and tumorigenesis in nude mice.

### Example 9

### Determination Whether BRAF Inhibition, INK4 Restoration, or Both Sensitizes Melanoma Cells to Chemotherapy and/or Radiation

Oncogenic BRAF may raise the threshold of apoptosis and cytostasis and thus contribute to the intrinsic therapy resistance of melanoma cells. Inhibition of BRAF may sensitize melanoma cells to therapeutic agents. Dacarbazine (DTIC), cisplatin and taxol (Paclitaxel) are commonly used drugs in the treatment of disseminated melanomas with response rate of about 10-20% (Bajetta et al., Semin Oncol. 29:427-45 (2002); Li and McClay, Semin Oncol. 29:413-26 (2002); Lang, Am. J. Clin. Dermatol. 3:401-26 (2002)). In vivo use of DTIC generates metabolite that methylates guanine residues in DNA at the 06 position, cisplatin covalently binds to DNA producing DNA cross-links, and taxol alters microtubule assembly (Li and McClay, 2002, supra). This aim is to examine whether BRAF inhibition and/or INK4A restoration will sensitize melanoma cells to chemotherapeutic agents. The identification of chemotherapeutic agents that synergize with BRAF inhibition and/or INK4A restoration should provide useful information for further evaluation in melanoma treatment.

### Methods

In vitro. The chemosensitizing effects of BRAF inhibition in 4 melanoma cell lines will be evaluated: 624Mel and WM35 (mBRAF het), A375 (mBRAF LOH), and Mel1363 (BRAF wt). First, the sensitivity of different melanoma cell lines to the same drug, and the effects of different drugs on the same cell lines may vary. Therefore, IC50 of each drug will be determined for each of the parental cell lines. Only in vivo experiments will be performed for DTIC, because it need to be metabolized in the liver to generate active metabolite. Dose-response curves and time courses will be determined to calculate IC50 of flavopiridol, cisplatin, and taxol in the melanoma cell lines in cell culture using cell counting and colony formation assay as read-outs.

In preliminary experiments, 624Mel, WM35, A375, and Mel1363 cells were treated with vehicle solvent and two drug doses around the reported IC50 (20 and 40 (M, and 5 and 20 ng/ml, respectively for cisplatin and taxol) of the chemotherapeutic agents (Mandic et al., Melanoma Res. 11:11-9 (2001); Merighi et al., Biochem Pharmacol. 66:739-48 (2003)), followed by daily cell counting and colony formation assays. Growth inhibition was observed after 6-24 hr treatment with cisplatin and taxol (both concentrations). Additional experiments will be done to generate dose-response curve and time course for the chemotherapeutic agents as well as for flavopiridol, and calculate IC50 values.

Next, similar experiments using flavopiridol, cisplatin, and taxol will be done in control and cells expressing mBRAF RNAi and or INK4A. The IC50 of control and RNAi expressing cells will be compared to determine whether mBRAF inhibition and/or INK4A restoration decreases IC50 of any of the agents in melanoma cells.

Cytotoxic and cytostatic responses are the main effects induced by anticancer drugs on cancer cells (Johnstone et al., Cell. 108:153-64 (2002); Schmitt et al., Nat. Rev. Cancer 3:286-95 (2003)). Therefore, further analyses will include melanin content for differentiation, FACS analysis of apoptosis/necrosis, and soft agar assay for transformation. Phospho-MEK by Western blotting will be used to monitor ERK signaling activity.

In vivo. DTIC and other agents that are sensitized by mBRAF inhibition and/or INK4A activation in vitro will be further examined in vivo using nude mice xenograft model. Mice will be inoculated subcutaneously on the left lower flank with melanoma cells as described above. One week after cell inoculation, when palpable tumors established, mice will be randomized into treatment groups (n=6 in each group) and initiate the drug treatment (group scheme as described for in vitro above). The control group will be treated with vehicle solvent. Drugs will be administrated initially based on published studies. Flavopiridol will be administered intraperitoneally (i.p.) at 3 mg/kg twice a week for a total of five injections. DTIC will be injected i.p. at 80 mg/kg on 5 consecutive days. Taxol will be injected i.p. at 20 mg/kg each day for 10 days (e.g., Li et al., 1999, supra). Cisplatin will be administered i.p. at 7 mg/kg for 3 consecutive days, two cycles of treatment will be administered at days 8-10 and 21-23 after tumor implant. Drug concentration and time course will be adjusted based on the initial experiments.

Tumor size will be measured twice a week during treatment and the observation period. The host toxicity will be evaluated by general appearance and body weight. Six weeks after the start of treatment, the mice will be sacrificed, the tumors isolated and body and tumor weights recorded. The control, single and double treatment groups will be compared. Tumors will be examined for markers of differentiation (melanin content and staining of melanocytic markers), proliferation (Ki-67 and PCNA), and apoptosis (TUNEL).

It is anticipated that suppression of mBRAF will result in inhibition of ERK pathway and increased susceptibility of melanoma cells to chemotherapeutic agents. Since flavopiridol, DTIC, taxol, and cisplatin have different mechanisms of action, the effects induced by them and the interactions with BRAF inhibition may vary and be dependent on drug dosage. Although cytostasis is the major effect expected for mBRAF inhibition by RNAi, apoptosis may also be observed when used together with flavopiridol and other chemotherapeutic agents. Depending on the results, additional melanoma cells can be screened to confirm the observed chemosensitizing effects. Treatment of cells and mice with more than two agents can also be done, for example, taxol, flavopiridol, and DTIC to increase tumor suppressing activity.

Expected Results

It is anticipated that, in cells expressing mBRAF RNAi and/or INK4A restoration, combined treatment with flavopiridol, DTIC or other agents may synergize to inhibit tumor growth and overcome the dosage limitation.

The above experiments will also be conducted using radiation therapy.

### Example 10

### Gene Expression Profiling for Identification of Downstream Targets of mBRAF in Melanoma Cells

The ability to specifically inhibit mBRAF by RNAi allows analyses of the effects and downstream targets specific to the mutation. Control and melanoma cells with stable mBRAF inhibition is an ideal system to apply high-density gene expression profiling to identify mBRAF target genes. This genomic technology provides the opportunity to get a more comprehensive view of the consequences of BRAF genetic changes in melanoma cells. Detailed analyses of these downstream target genes will help to delineate how mBRAF causes aggressive behavior of melanoma cells and how suppression of BRAF mutations induces growth inhibition. The identification of mBRAF target genes in melanoma cells could also facilitate the development of novel therapeutics that specifically target these molecules.

### Methods

The Microarray Facility at Mount Sinai provides consultation, instruction, assistance and expertise for the high-throughput gene expression profiling. 624Mel control and mBRAF RNAi cells (grown in regular media) to the Facility for gene expression micro-array assay using the Affymetrix human genome U133 GeneChip. Detailed analyses of the micro-array data is ongoing. Genes that are differentially expressed in the 624Mel control and mBRAF RNAi cells are potential mBRAF targets. The target genes identified will be compared with genes found to be potential markers of melanoma progression in reported expression profiling studies of melanoma samples (e.g., Carr et al., Oncogene. 2003; 22:3076-80; Hendrix et al., Oncogene. 2003; 22:3070-5).

The biological data described herein show that inhibition of mBRAF in 624Mel cells not only induces growth inhibition, but also triggers cellular differentiation. Therefore, the mBRAF target genes may regulate either growth or differentiation, or both in melanoma cells.

Preliminary Results

Consistently, preliminary analyses show that several genes involved in cell cycle control, cell growth, and differentiation are potential targets of mBRAF (Table 3). However, these need to be confirmed.

TABLE 3

| | | Reading: | | | |
|---|---|---|---|---|---|
| Gene class | genes | RNAi/control | mBRAF | Fold ↑ | Fold ↓ |
| Cell cycle | Cyclin D1 | 425.9/686.7 | | | 1.6 |
| | Cyclin A1 | 16.3/35 | | | 2.2 |
| | CDK3 | 7.7/35.2 | | | 4.6 |
| Growth | SCF | 0.5/1.6 | | | 3.2 |
| | FGF 17 | 5.2/30.1 | | | 5.8 |
| | MMP1 | 1.2/12.3 | | | 10.3 |
| Differentiation | CEBPD | 132.8/59 | | 2.3 | |
| | MEOX2 | 16.1/2.2 | | 7.3 | |
| | LHX2 | 23/4.5 | | 5.1 | |
| | HOXD1 | 129.2/64.9 | | 2.0 | |
| | HOXD3 | 53.3/32.8 | | 1.6 | |

Putative mBRAF target genes deemed most likely to be involved in mBRAF RNAi-mediated growth inhibition identified by a GeneChip will be further characterized. Particular attention will be paid to known regulatory genes or key signaling molecules. Western (if antibodies are available) and Northern blotting, and quantitative RT-PCR will be used to confirm different levels of gene expression between control cells and those expressing mBRAF RNAi. If confirmed, it will be determined whether the genes are similarly regulated in other melanoma cell lines by comparing gene expression between control and mBRAF RNAi expressing cells by Western and Northern blotting, and RT-PCR. The roles of these genes will also be examined by transfecting in constitutively active or dominant negative versions (if available) or by RNAi and over-expression approaches to recapitulate the mBRAF phenotype.

To further delineate the regulatory mechanisms, melanoma cells can be treated with MEK inhibitors PD98059 or U0126. The expression of these confirmed mBRAF target genes can be examined in control and PD98059 or U0126 treated cells. If the expression levels are not affected by PD98059 or U0126, the genes are likely regulated by mBRAF through mechanisms other than ERK activation. Characterization of the alternative mBRAF signaling pathways should provide further understanding of mBRAF activity in melanoma cells.

Pavey et al. have recently published results from a compared a pool of different melanoma cells with and without BRAF mutations. (Oncogene. 2004;23(23):4060-7). Genes which were shown to be differentially regulated will also be evaluated for BRAF regulation.

### Conclusion

In conclusion, activating BRAF mutations and loss of INK4A expression occur at high frequencies and often co-exist in melanoma cells. Several melanoma cell lines were identified that harbor both BRAF and INK4A lesions. A highly specific RNA interference (RNAi) approach was then developed to inhibit the expression of the T1796A hot-spot BRAF mutation. INK4A wt cDNA expression construct was also generated to restore functional An INK4A expression. Although knock-down of mutant BRAF or restoration of p16^{INK4A} significantly inhibited the growth of melanoma cells, neither generated full inhibition. Surprisingly, simultaneous inhibition of mBRAF and restoration of p16^{INK4A} in melanoma cells resulted in potent, and even lethal effects, in melanoma cells. These data suggest that BRAF and INK4A lesions cooperate in the malignant growth of melanoma cells. These findings can be extrapolated to other tumors which exhibit both activating mBRAF mutations and mutations causing the loss of functional p16^{INK4A}.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims.

It is further to be understood that all values are approximate, and are provided for description.

Patents, patent applications, procedures, and publications cited throughout this application are incorporated herein by reference in their entireties.

What is claimed:

1. A method for inhibiting the growth of a tumor cell that comprises an oncogenically activated BRAF protein, and is defective in the expression of a functional p16^{INK4A} protein, comprising:
(a) inhibiting expression or activity of the oncogenically activated BRAF in the tumor cell; and
(b) restoring functional p16^{INK4A} activity in the tumor cell.

2. The method of claim 1, wherein the inhibition of oncogenically activated BRAF comprises inhibiting expression of the BRAF nucleic acid.

3. The method of claim 1, wherein the inhibition of oncogenically activated BRAF comprises inhibiting expression or activity of endogenous BRAF polypeptide.

4. The method of claim 2, wherein the inhibiting expression of BRAF nucleic acid is by RNA interference using an RNAi specific for BRAF.

5. The method of claim 4, wherein the oncogenically activated BRAF is a mutant BRAF.

6. The method of claim 4, wherein the RNAi is provided to the cell in a viral vector.

7. The method of claim 6, wherein the viral vector is a retroviral vector.

8. The method of claim 1, wherein restoring the functional p16^{INK4A} activity comprises introducing into the cell a nucleic acid sequence encoding a functional p16^{INK4A}.

9. The method of claim 8, wherein the nucleic acid sequence is cDNA.

10. The method of claim 9, wherein the cDNA is provided to the cell in a viral vector.

11. The method of claim 10, wherein the viral vector is a retroviral vector.

12. The method of claim 1, wherein restoring the functional p16^{INK4A} activity comprises contacting the cell with a functional equivalent of p16^{INK4A}.

13. The method of claim 12, wherein the functional equivalent of p16^{INK4A} is a cyclin-dependent kinase inhibitor.

14. The method of claim 13, wherein the cyclin-dependent kinase inhibitor is flavopiridol.

15. The method of claim 1, wherein the tumor cell is a melanoma cell.

16. The method of claim 1, wherein the inhibition of BRAF and restoration of p₁₆^{INK4A} activity is simultaneous.

17. The method of claim 1, wherein the inhibition of BRAF is performed prior to the restoration of p16^{INK4A} activity.

18. A method for sensitizing a tumor cell to the cytotoxic or cytostatic effect of a chemotherapeutic agent or radiation,-wherein the tumor cell comprises an oncogenically activated BRAF protein, and defective expression of a functional p16^{INK4A} protein, comprising:
(a) inhibiting expression or activity of BRAF in the tumor cell;
(b) restoring functional activity of p16^{INK4A} in the tumor cell; and
(c) contacting the cell with an effective amount of a chemotherapeutic agent.

19. The method of claim 18, wherein the inhibition of BRAF comprises inhibiting expression of a BRAF nucleic acid.

20. The method of claim 19, wherein the inhibition of BRAF comprises inhibiting expression of endogenous BRAF polypeptide.

21. The method of claim 19, wherein the inhibiting expression of BRAF nucleic acid is by RNA interference using an RNAi specific for BRAF.

22. The method of claim 21, wherein the oncogenically activated BRAF is a mutant BRAF.

23. The method of claim 22, wherein the inhibitory RNA is provided to the cell in a viral vector.

24. The method of claim 23, wherein the viral vector is a retroviral vector.

25. The method of claim 18, wherein restoring the expression and activity of a functional INK4A comprises introducing into the cell a nucleic acid sequence encoding a functional p16^{INK4A}.

26. The method of claim 25, wherein the nucleic acid sequence is cDNA.

27. The method of claim 26, wherein the cDNA is provided to the cell in a viral vector.

28. The method of claim 26, wherein the viral vector is a retroviral vector.

29. The method of claim 18, wherein restoring the functional activity of p16^{INK4A} comprises contacting the cell with a functional equivalent of p16^{INK4A}.

30. The method of claim 29, wherein the functional equivalent of p16^{INK4A} is a cyclin-dependent kinase inhibitor.

31. The method of claim 30, wherein the cyclin-dependent kinase inhibitor is flavopiridol.

32. The method of claim 18, wherein the tumor cell is a melanoma cell.

33. The method of claim 18, wherein the inhibition of oncogenically activated BRAF and restoration of p16^{INK4A} is simultaneous.

34. The method of claim 18, wherein the inhibition of oncogenically activated BRAF is performed prior to the restoration of p16^{INK4A}.

35. A method for treating cancer in an individual having a tumor comprising an oncogenically activated BRAF protein, and having defect in the expression or activity of a functional p16^{INK4A} protein, comprising:
(a) inhibiting expression or activity of oncogenically activated BRAF in the tumor cell; and
(b) restoring functional p16^{INK4A} activity in the tumor cell.

36. The method of claim 35, wherein the inhibition of oncogenically activated BRAF comprises inhibiting expression of the BRAF nucleic acid.

37. The method of claim 35, wherein the inhibition of oncogenically activated BRAF comprises inhibiting expression of endogenous BRAF polypeptide.

38. The method of claim 36, wherein the inhibiting expression of a BRAF nucleic acid is by RNA interference using an RNAi specific for BRAF.

39. The method of claim 38, wherein the oncogenically activated BRAF is a mutant BRAF.

40. The method of claim 35, wherein the RNAi is provided to the cell in a viral vector.

41. The method of claim 40, wherein the viral vector is a retroviral vector.

42. The method of claim 35, wherein restoring the activity of a functional p16^{INK4A} comprises introducing into the cell a nucleic acid sequence encoding a functional p16^{INK4A}.

43. The method of claim 33, wherein the nucleic acid sequence is cDNA.

44. The method of claim 43, wherein the cDNA is provided to the cell in a viral vector.

45. The method of claim 44, wherein the viral vector is a retroviral vector.

46. The method of claim 35, wherein restoring the functional activity of p16^{INK4A} comprises contacting the cell with a functional equivalent of p16^{INK4A}.

47. The method of claim 46, wherein the functional equivalent of p16^{INK4A} is a cyclin-dependent kinase inhibitor.

48. The method of claim 47, wherein the cyclin-dependent kinase inhibitor is flavopiridol.

49. The method of claim 35, wherein the tumor cell is a melanoma tumor cell.

50. The method of claim 49, wherein the melanoma tumor cell is 624Mel, WM35, or A375.

51. The method of claim 35, further comprising administering to the individual an effective amount of a chemotherapeutic agent.

52. The method of claim 35, wherein the inhibition of BRAF and restoration of p16^{INK4A} is simultaneous.

53. The method of claim 35, wherein the inhibition of BRAF is performed prior to the restoration of p16^{INK4A}.


SEQUENCE LISTING (33 sequences)

SEQ ID NO: 1 - DNA, 50000 bp, Mus musculus
cttttttgacagccaaatctttttttattgggggaacgggtctctagggggtaggcctaggccctcactgcacagcttgttcattggcactgcctccagaatcctgtggcttcatcacatctggaagctcgggagggctggagaagggctcaatgcggagagtttcgaaggtgtcatcttctcggaaggccaggcccactgtggctgtgctgtctggctagtgaagccacactcgcccagagttttgccatcatcaaggagctggtcatccttgtaaagcagccgctcctctggcggccgcttgaggatgtcctcgacgatgtgcttcaattcgaacacagtgctcaacttcttggcatccagaaagatggtggtcttgtggcaccggatcatgagaaacatgtccattctggcggctgcttctggcttgaggcgccagtgcagccccaattgtggctttctttgtttcttttttttttaaagctttatttatttattaattatatgtaagtacactgtagttgtcttcagacaccccagaaaaaggtaacatctcattacggatggttgtgagcaaccatgtggttgttgggatttgaactcaggacttccagaaaagcagtcagtgctcttaagtgctgagccatttctccagcccaattgtagctttctataatggtgtctgtctgtagcaaagaaaggcttctttttattgttattatatattgtatatataaaatttttctttttattgctattatgtattggatatataggattgtatatatatgcatatgtagttatatatttatatattacatacatacatatatatagttgttataattttattttatgtttatggatgttttgcctgcatgtatgtctgtaccgtgtgtgtgtgtgtgtgcgtgtgtgtgtgtgtgtgtgtgtgtgtgtgtgtgtatctggtgcctgaggaagtgagaagagggtgtcagatcccctggaactgtagttatatgttgcgcgcgctcaactggccaggaagaacgacgctgctacaggatccttctgcacacatttattcagtcctgtttcttctttctccatatatctcccttgtttatatctcccttgtttatatctcccttgtttatatctctcccttgtttatatttcccctgtttgtatctctccctcgtttatatctcccccttgtttatatttcccctgtttgtatctctccctcgtttatatctcccccgaaccctgggcctctcactctttttatactctctctcatccacgcactgcaggccacgccccctcgccagtcacgaggcttcagctaatcagggcagcaggggcaaatctccaccaaattggattcacctgtatcctggtacacctgcgcagcactcaagatgtttgtgtcttatatgaggaagtcaggtgcaagtcatatgacttagctgcagtccctggcgcctttaggactgccgccacacctgctcctaacaattcccccttttttcttttttggcagagagaatgcctgtagatagccccccgcagccatgccccttacccgtccttgggtgacaaacagcattggtttgatccctgtcttaggttggtgacatgcccagggagtcttatcactgactacctctctatcatgccaagccacacctggggagattgtgttttgcttgcggcaggtgcatcaaaaggccaggatgttaattaacctatggccagatcttaattgctgcaagcaagcctcatgggtgaaggtagaggtctgatccccagtgtgcaagcataggggccctacacaaaaccatcccttaggctcatcacccagagaggtcttggccagctcccgtgtcgtttttcctgggggaagggaactaggacactgaaccttcatgcaatcagacatgccttccacaggatgccaacagcaagctatcctctgtgcagtgcttagcctcgcaccccacggcataacgcagcataatttcttttagagcggcaaaccgaatctgaggagattgtcccgcctccactgcagcaaaagcctacgctaccatggcgaaagtgcgggtcgcacactctgcacctaacacatgtgccaaacacaaaacacacacacactataacaagcatacatcccagggctctcatccccactcatcttccctgaagcaagggatagatagccagaggggctatctctttaagaggaattcagggatcaaaaagcgtggaaaaatttgaatgtcatgtcgagctggtatcggcttctggaataccgaaaggatctcccatctctgctccatcctttgtgcttgtgggatcatccaccacatccacaggggcaactggatcaggagcctcattcttgcagtgtctcaccaatctctccggcacccaaagaggttccatctggtcctgtggaaacacacaaacagaccctctcgccctcgtcaacaccggatctgctcgatccccaggggagatggacacaatacctcgtggtaatgagaccataacctcaatcacctgtgtgttcatctggggagtcaatacgccaaatctccggacagaaggtccacctataggtggctgctgaggaggcatagggaggaggcacagaagctaattcgccttcctcctccacctcggctcttttattttgtcctttctgtccacctgataacactgtgtctcctttctttttcctttttctttttaagcccttctccttttccgacatactttcttgttgctctgtaaggattttctgacctgtcttgactgcctctacacacaaacagtaacagagtccatatatcagaacaaacaagaccaaaactatcagacctacccacaaagggtcaatgggagaaaaaagcataactacacagcgaggatctattgatcactacactgagcttatcatagttccttaacttgtcccaaagccaggaaccttaacttgtcccaaagccgggaacctttcgttaccttgtgcctgcttgctggcaactttatgctcacctctattttatcagaggtccttcccaaactcctgggttactttgtgcctacttcctggcaactttatgcttgcctctattttatccgaggtccttcccaaactcctggggttgcaagtttcactcaccgtgaacttaccctgcctgccggcaaccactgactgctgaaagttctgaactcggtgggggagtcggttccccgtacgggccaccaattgtcgcgcccactctcgaccagcaagaacgacgcgaccaccagtccttctaacagcagtttattcagtcttcatctctcttctttctcttcatcagtaccgttccccagctgaagagttctgaatccacgccggatccttctcaacagtctgtttcacgggaacctttattaaccgctccttccccgtgatgcagttctgaatcctccctgtagcagggggtcttcgctcatgcctgaagatgtttcttttcccgggtttcggcaccaactgttgcgcgcgctcaactggccaggaagaacgacgctgctacaggatccttctgcacacatttattcagtcctgtttcttctttctccatatatctcccttgtttatatctcccttgtttatatctcccttgtttatatctctcccttgtttatatttcccctgtttgtatctctccctcgtttatatctcccccttgtttatatttcccctgtttgtatctctccctcgtttatatctcccccgaaccctgggcctctcactctttttatactctctctcatccacgcactgcaggccacgccccctcgccagtcacgaggcttcagctaatcagggcagcaggggcaaatctccaccaaattggattcacctgtatcctggtacacctgcgcagcactcaagatgtttgtgtcttatatgaggaagtcaggtgcaagtcatatgacttagctgcagtccctggcgcctttaggactgccgccacacctgctcctaacagttatagatggttgtgagcctttgagtggggactgggaatcaaacttgtcctctagaagagtagccactgctcttagatacttagccatctttctagcctaaagaattttttttccctttggcttttcaatacacggtttcttggtgtagccctggctgtcgtgtcctgtacacacacatgcacatgcacacgcacacatgcacagtcatccatgatggagagggggactgagccccgggcctgagatgccaagcacacacactgtcattgaactgtacctttagtcactaaaaagccctggtctgacagccactgtgccgctgggcatggtggtgcaggcctttgactccagcacttggaactcagcgtctggctgacctctgtgagtttgctgcaagcttggtcaacatacagagttccaggccagccagggctacatagtgagtgtggctgtgtctcaaatgtaaacaaaaaggccttacacaaccaagtcaaactcaaccaccctttcttactgttttggtgtaagtgacaggacctcactttgtgagaagctgtcagctgttgccctaataattaaggttgaagtctatcattgtctgggtagccttctgggctccatgctaatggtgaactttctctgtcagactctcttcttaacctgggggatccctgtgatggtttgtatgttcttggctttatctcctgggattaaaggcatgcactaccttgcctgggcctaagcttttcatagctgctgtgcctcaagatctccatgtcaagatctaggtcagaaacttgtgtcttccagcctcaagatctggatcacatgtgagccctccaattctggattgtagttcattccagatatagtcaagttgacaaccaggaatagtcattacaatccaacccttgtcttgtttgtttttttgtttgtttgtttttgttgttcttgtttttttcggtttttcgagacagggtttctctgtgtagccctggctgtcctggaactcactctgtagaccaggctggcctcgaactcagaaatccacctgcctctgcctcccatgtgctgggattaaaggcatgcaccaccactgcccagctaatccaacccttgtcaatttgacacaaatatctcatgtccacatgaaacaataacaagatcataaatatgcctaacatgatataactattccttgtacaatcacaaaaacatttgtaaaattacagtggggcaatgtccctcgggaacattcttttagtatctcaacttaaatacagattgatgttaaaaaaaaaatgggagaaagcacaaatagctatacaaatgtgttcttaacaatataaaccagaagcattgatattactttataatcctcatttctgcaactggtcatgtggtcttagatggtatttataactacctccctctactacccattctgtattttctccatcctctgcaagcacctcagctggtcttcttggctcttttcctggaggagtgacccataccttcacccctgatgggtctgtgtcctttgtcatcctgcttggattaggctgttttgttttctattgactttaatcacaggacttggtagtactaggagacaccctaagggatctcctgcactccagacataatcccttttaccttcattgtggtagttgatccaatttccccatggtaatctggatctatcacccctcctaacactgttattcttttcttagcctgttggtttaagggcattagaagcccaaaatgacaagggggaagtttgagcttccagttaaatgaaatgttagttgtggctcctggcaggaacacactccactcctggtaccaatttgtattagtcaggatttgtcagagaccataccgtgagaaatcgagcttctcacaatctcccaccctgacaggccaaatggcctcgtgataggagccggttgtcactccctcctccctgttcccttcctggcacctgaggctgtgaaagctgaattatagtccccgcttccctatctcttcctgactccatgacatccaaggacatgagttacacctgagcccggcctgacacctcaaggctgttaaggaggatctatgttctggagataagatgcagagtgcccaccgcctggagcctgactttggcccttatgtcagcagatgtccacttgtttgttctttgttaaattcccccttgacccctccctattccccaagatgtatgctttaaaaccaggcatctcagtataatagatggagaccttgataggcacccttcttggtctccgcttctccttcccttcttcccattttcttccaggtttgcggtccctctcacgaataactgaatcctgcgggacgggataagtggcacccaacatgagggtgaggattgtatttccttccagtggaggttccagagaggtttgtcacgaccccaagaatttagaagtagtgaaggaccccttctgccgctcacggaagagtgagaagtccttggtgagttgagtcatctccacttcaggttatgggaaataagctttctaaagaggcagccttcatcaaaggcttaaagatagctctcagggaaagaggagtacgagttaaaaagaaagatttgataaacttttattttcatagaccaggtatgtccatggtttattatagatgaagcagagatacgttgtaaaaaatggtgaaaggtaggtagagacttaaatgataaactagctaatgagggtcccgatgcggtccctgcaactgtcttttcttattgaggagtaaatatcagaagctgccactgtgcctcctctcccttctctggcagaattcccagaagaaggagataaagaattagagtctgaacatgagaaagagaaaataagtttcagaaaagcagttatcccctgtttgggaccttttagcaaaaaagagggaaaatgaaaataagctatatcagagctctcctagggacagaaggaaaatgggagacgtcctgcttcctctctggctcctatggagatattcctagttctggttaggatgtctgggtccctttgagacatcaagttctattccctgtctgtctattgtctggttttggtgcaccaaaatgtgtccatatatctgtctatttatgtttgcttttgttttgttgtttgaatgatagttgtactgtgtttcatgttgaaaaaaatggttaaaattttatctgctggctgtccattctttgatttagtttaacttgtttaaaaagcagtctcaatttgcacagcagaaacagataactgtggctgggagtcaagtatagctggaagagccctgctgagagccgggacgggataaggattctctagagtcacagaatttatggtatgtctttctatattaagggaatttgttgtgatgacttacagtctgtggtatagctaccccaacaatggtcagctgtgaatgggaagtccaagaatttagtagttgcccactccctcaaggttagtgaggctagttgttgtagctgatcttctgtagaagtagattccaacagatgtgttggcaagtaaatgcaagcaggtgaaggagagcaaatcttccttcttccaatgtctttacgtaggtctccagcagaaggtgtggcccagattaaaggtgtgtaccaccatgcctggatgggatttgttttatcctaggatgatcttgaactcagagatctccttgctttagtctcctgggattaagggcgtgtactaccttgcctgggcctaagctttgctttgctttgcttttttttcttttttcttttttcttttggtttttcgagacagggtttctctgtgtagccctggctgtcctggaactcactctgtagaccaggctggcctcgaactcagaaattcgcttgcctctgcctcccaagtgctgggattaagggcctaagctttttcatagccactgtgcctcaagatttctatgtcaagatccaggtcagaaacttgcatcttctaccctcaagatctgaatcacagatgagacctccaattctggattgtagttcattccagatatagtcaaattgacatccaagattagccattacagtcccagacctcacactctatacctgcaaatggaatgccattccttggttaagatcataggttcagcttcctttctcctggtagaacccattaagctagaacctgagattcctagagtccttccctatggtaacgaggcatctcagtaccttaagccttcagctaatgacttttgcttaccctggatattcctaccccttgacctaaaactatataaaccttgaatcaccccaagttaagttgatctgtctccctgaagctggtcctggtgtgcccttattactcactgggcttctggacacctctcccaccctccacaccctttctaaccatcatctctgctcctgggaggggacaggtgcagggagggtcacatttagtcttcttgcctcaaccttttgaatgctgtccaccgcctgcctcaccacatctgtcatttcttcttttgcaatatatgtagttgaactcaaaatttccctgttagtacgactttcctgcacacacatttggattgctaggttttttttactttagtagttttatcattacttcgaagccctatgaagatatttatgttcttccttggcccctgggtcatctctcagccaggtccctaggtacaacctctatattataggacccaggctccctgtctggtttcactttcttaacctttgaagttatcattctaggtaatatttaaaaaaaatagtagtgcactaagaggctgggtgtggtggctcatacctgcaatcccaaccttcaggctaagacaggaggattactgtaaattcagagtcatctgtgggatacacagggaattccaggtcaatctggtctccagagcaggcaggcctacacagagaaactttgcatatatatatattttaaaagaaagaatggaaggaaggtcagaccacattttattagtgagcttgggaatctgcatgctttgtgactgctggatgagatgtgatagtgtatcctaaatggatggattttatcatcttttcttccagaacacaaacagctgcttttcatcttcctgcttgcctagcttctttgggctagatggttctttgtagctctctacctttaaactctaccccaggtaaagttccgagtagtggcctatctttatgttgatcaatgtttgatcatccctgaacagagagagggaatcccctctgtggtttgtttgtttgtttgtttgtttgtttgtttgtttttgttttacaagacagagtctctctgtgtagcactggctgtccaggaactcactttgtagaccaggctggccttgaactcaaagagatccgcctgcctctgcctccccaagcactgggattaaaggcgtgcgccactaccaccaccacccagctccctctgtgttgtaaaacagctgctcctgttgctttaaggctgaggcaaggcgctacactggggaagagaaagaggcaaaggtgaataaagcaagataaaactgccatagaatttttcaggccaactttttttttgtttgcctggttgctatggacttttatctatttttttttttgagtgcctacaaagtttgttctgacatctcagcttattttatcagtgtttctatggagagatactatcgtagagcttccttgtttactgaattcctgatgtcactcctgaatggcgtattttaaaaatcattatttactgatccttcgggaccacaagataagtgagaactccagatattcagtctctctgtctaaagcacaagaggtgggcaagactaggttagcacctcgactgtgcacgttgcatttaatgcagatgtctggaagcagatcacagagccgctgcctgggacatgcacggtggtcagcagagataatgttccctgcctttcacatagacctccaaactctgaatgctgctggagaacagagaggtcagttaagtgaactctcttcacaccccagggcctgcttgaacagctttcgttaaagaactaccaagcaaacaccctggtcccagggcactgccttgccccaaccccaaactgccccctgacatagtatgaaatgctgctctgggctgcagactgactacactgtagccagagataattgtatgaataataaaaacaaattaaaaagagatattatggctggctgtacagtttagtagtgtagcacagacttaccacgtgcaagtccagggttcaaccgtcagtaccaaaccctccctccacaaacccggaggtatcttcttatcataccgcagttgcttggttagtaaagcctggaaaatataatacataactataaaagtgtgatgagacactgttatgattaggtgagtatatgatgagacactgtgcttgctggcatcactgatgtctggcctgtgagagttgagactgacccttagctagataccagtaacagattctgataattgtctgataatcctcatttagcccaaggtatgaaattgctaactgtgcacttcagagtcataaatacatttaaaatcataggtttcaggtctgggaacacatttgaaggagacacattccaaaaataaaaaagaagagggggaggagagatgagggggagaaggagggagaagaggaagaggaaggagaaggaggacatgtcttagacataaaggggtcaatgggaacttatttataatcatgaaatcttggcaaccagcccgattttcaatgatgggagagtagacccctctgctgctccatggatgataattccaaataaacatggtgatcagagattgagggcaatggagttttaaaatcaagggaaaaacagcaagcgtgcatgcttgtgtcacttgttcgtgactgaactagtgtgccctggcaaggcctacagggacccccacacaggcagtgatgtgagaccaggtgaccctccagtgtgactgtgtgtttctcatgcttttgggggcttcagaaaagccctcagaccaaggatctggacctcatctctctggagtctggtctggggacagctggacagccctcgtgagaactgatgtggagaaggcagggctcaatgcccctcactggggctcttggggttttcatgtggcagcagtatctgtagaccaggctggctttgaactgcctgcctcttcctcctgagtgctaagattaaaggtgtgcaccaccaatgttcctattccaggaatgtcctcaatcaatgacttgtaagtgtggatggtgccacacccttatccactggtggggatcccctagggcgggtccccttatgtcctggaggtctcagggacaggtgatattcttgaatccatcttgatcatcacatctcatggttctcagtctgctcagccctctgtcgtcacctgaagtcatcttccaaataaccctgattttcatttgtcctaggatacttgtctcagggtctgcatctggagaaatcacatgagaacatttgggacaagacaagaagaggactgggtggcatccagggagcaacaagggaagcaggtgatgttgtgtggcccagggcccttctcctcagcctctcttgttccctgcctaagcttgggcggattcccctctgagcccacccgagcccctgggacactggtggaactcagtaggagcccctccctgcagctgtctcaacaggtagctgcatgagtggccttgaagcaattatcagcaattcagccctggcaatagaggccaaggtcctggcctgtcttggtgatagcaagagcccaaggaaagactggaagtttcctactggaaagaagcagaggatgaaccatgtacctgggcccaggttgggtgggacttgccactcagagcccctaaccagggttgttcagaggactaggccagggccaggaccaagaaagggatagaacgggcatgaggaggaagggtgaagggatccaaggaatctctggtcctgttccctgttaggacatttgtcatggaatcactctcgcttagtgtctctgttatctgggtgctaatagcaactattcagttgctaggatgttaggtgagtctgaacctacccttgatgttgatctgaagaggcgatgcgttagactgcaggttggaggccaagtccaggacagtgttgatattctggatctccaagaagcctccaaggccaaagccaggccagtgtctggtctcgcagaggaacagctctgcatctcttgcccggttggctctaactaccacattagacttcagttgcgtcaaaaaacgaggggaccccagcgccttcactaggaagttgacctcagaaggaggagatggaatggcaccatctgatgtaagggaagagaaaataaattattaaccagtacggcccagtcctattggccccatgacagacgagggttatcactaagaggaggaagctgccttaatgtgcaaactcaggggccagtcctcagcttccccggctgtctccaaggcctggtcctgcttttccttgatcacttcctggctctgggatggcagctgcctggcagggatggctgctctgggccctgctcctgaattcggtgagtctgttgcttgtggctactccttggtgcctcccattagggcaaagtgatacctgatacctgatactgggtacctgataacggggaagggtcccacggctgtgggagggttcctatgcccaaagataagtgctggtggaggggtctccaggtcaaggggttgaagggatagaggtcagagaggcaaagggatggggcctttgtctgaggttaaatggggaccaagtcaggtgctagaggtggatcccagtgaacagcgcctgaaatattctgggcttgggaggaggttttgctaccatccttgtttgctctcaggcgatagcattggccaatgcaggatgtaggagtggggggctcttatacagactcttgtacaaggaaccctgacctcggggtagagctcagcctggagactcaaactgacagcaataaaggtcgctatctcctactctcccctgcagcacgaccctttaaagccacactctattggatcacttccttttctgaatagccccctcactgtccattgggggagtgcccctccattggcaccctaagcatagcacgagcccccacaagcctcccgcagcactcccagccccttactgctggccttcttacccatagactccctagcctctcactctccagacagtccctggctgtgccaaccagccttagggcttatggatgctatcggttctttctgcaggcccagggtgagctctacacacccactcacaaagctggcttctgcaccttttatgaagagtgtgggaagaacccagagctttctggaggcctcacatcactatccaatatctcctgcttgtctaataccccagcccgccatgtcacaggtgaccacctggctcttctccagcgcgtctgtccccgcctatacaatggccccaatgacacctatgcctgttgctctaccaagcagctggtgtcattagacagtagcctgtctatcaccaaggccctccttacacgctgcccggcatgctctgaaaattttgtgagcatacactgtcataatacctgcagccctgaccagagcctcttcatcaatgttactcgcgtggttcagcgggaccctggacagcttcctgctgtggtggcctatgaggccttttatcaacgcagttttgcagagaaggcctatgagtcctgtagccgggtgcgcatccctgcagctgcctcgctggctgtgggcagcatgtgtggagtgtatggctctgccctctgcaatgctcagcgctggctcaacttccaaggagacacagggaatggcctggctccgctggacatcaccttccacctcttggagcctggccaggccctggcagatgggatgaagccactggatgggaagatcacaccctgcaatgagtcccagggtgaagactcggcagcctgttcctgccaggactgtgcagcatcctgccctgtcatccctccgcccccggccctgcgcccttctttctacatgggtcgaatgccaggctggctggctctcatcatcatcttcactgctgtctttgtattgctctctgttgtccttgtgtatctccgagtggcttccaacaggaacaagaacaagacagcaggctcccaggaagcccccaacctccctcgtaagcgcagattctcacctcacactgtccttggccggttcttcgagagctggggaacaagggtggcctcatggccactcactgtcttggcactgtccttcatagttgtgatagccttgtcagtaggcctgacctttatagaactcaccacagaccctgtggaactgtggtcggcccctaaaagccaagcccggaaagaaaaggctttccatgacgagcattttggccccttcttccgaaccaaccagatttttgtgacagctaagaacaggtccagctacaagtacgactccctgctgctagggcccaagaacttcagtgggatcctatccctggacttgctgcaggagctgttggagctacaggagagacttcgacacctgcaagtgtggtcccatgaggcacagcgcaacatctccctccaggacatctgctatgctcccctcaaaccgcataacaccagcctcactgactgctgtgtcaacagcctccttcaatacttccagaacaaccacacactcctgctgctcacagccaaccagactctgaatggccagacctccctggtggactggaaggaccatttcctctactgtgccaagtgagtagatctgaggggaacaggtgagagctgctatgcccccaggaaccaggccagaacctagctccacccttgggagccagggacagctcgtatgtgcacatatcagggccatggcctgtccaagtctatttaagtcccttcttggagctcactcccatcttattcctgcaggaattttgtcctaccagtctttccagctccaatccatatgatctttccatccatgatgctcctggtatcaacttaataatttttagaattactttaacttcacatgaatgaatattttgcttgtgcatatgtatacgcactgcttatatatgtgcctggtgctgaagaagccggaagaagttgctagattttcaggaactggagttgaggtcagtcatagcggccaggtgggtcctgggaaccgggctctggccctttgcagaaataccatgaaacgtctgcgtcctctctcctgccctcatgtagtcttagtttaaatctcaaagcgatgtctcaggtagtgtttgtgtttgtgatgcttccttttctggactctgtgtcttggtctgtagggactttggtagcctcacaactggctagaaatatgttcatctgggcttaggtggaactgtggttagtctccagtcccaggcatcagcacagttttttctacaaccttatgctgttgaggttctgcttctggctttgtccattttggctggcacaaacaggatgccagtgagctcatcagacagagggaaggttggtgagagggccagaggtagaggaggctcctggagaacatcatggagagtgaagtgcctcaaatggccttgtccactctagagcaggcgagggttacagcaggtaaccacagctgagtgttcttatgaaaacagttttgaccctgcaagccccagacttcatagtctttagagccatcagatgagagcagaaagcttttgctggctctcattgctactggctgtctatccccgtttgagtctccagtgcaagctacttcctagagtatccatgctgtcccctagatcggacagcagagaagggctgtggagaggcatcggggatcagccacgcaaaagacaatttaaaaaatattatttatttttatttatacaggtacactgtacctgtcttcagacacaccaggagagtacatcagatcccattacagatggttgtgagccaccgtgtggttgctgggaattgaactcaagacctctagaagagcagtcagtgctcttaacctctgagccatctctccagccttacagtgtagcttttgtttttgttgcttttatgttgtttttagacagagtcacactatttgagacggtcctcaaattcacgcccttgcctcagcctcctgagggctgtggctgcaagcctaagccatcatacttggcttgtactacctttattttgattttgaatgctcccgactcctggtgagtcagttatgttaattctatagactggaaacctgaggctcagagtggtatggtaagacgggcaaggccacacaggaatccagctctctttgacggctctgttgatcaatatactcacttgttcagacctcagaatgtgtataaagagcttggtgttggtagtctattcagtctccacagaggtgtgctctgtagaaagggtttagttgaagggcacaggcccctgtccaagggcattctctgggtctgtgagctccagggctcaactcaacatttaggggtgattctagctctgggaggggaaagtgaagaacagcattgagatctgtgagggagatgggcatggctcagttctgggctcatcacttaatggtgatgctcatttgacaggtctggaaggtttggctatgtgagggggcataggaagcatcacctgcccaagggaaccacattcagtggactaggggaccatatgagactaccttgtgaggagatagtcattttgaactctctgggcctggtattgtggagacactgctcctccaatagcggggagaggagctggggcagggaggggccaaagagtccaggcagggccaggaaaggttctttccctttgtggtttccccctagtgcccctctcacgtacaaagatggcacagccctggccctgagctgcatagctgactacggggcacctgtcttccccttccttgctgttgggggctaccaaggtaagtgaggtagctgggggggctactgaagggataattttggcacagagataataggtaggaggagggagaagccatggtgagtgtatccaggatctgggggcctggcataagggggctgcaggcaatgcttcctacctcactgctctcatctctcaatgctacccaggagctctggttttgtgcctttggctgggaaagggaaatgaagcatgggataaggctgttattggagtgaggaagcaatagaaggacaggaatgggagaaggttacaccctgaggggaggaggggaaaagggttcaacaggagggaggctcagggtttctcttcccagggacggactactcggaggcagaagccctgatcataaccttctctatcaataactaccccgctgatgatccccgcatggcccacgccaagctctgggaggaggctttcttgaaggaaatgcaatccttccagagaagcacagctgacaagttccagattgcgttctcagctgaggtaggggccctgcagagtccctggttctatgcttgcaatccctaatggtgtgggtctattccagtcaaatctacaaactggctctacttgttcctgactggccccgggcagtgaacacctgtgcctagctgtggcgcttgtgttagaggctcctgcagttcattcctagagtgtgtggccactcagtatgtggtccgtgagctggctgtgtgcttgcagcgttctctggaggacgagatcaatcgcactaccatccaggacctgcctgtctttgccatcagctaccttatcgtcttcctgtacatctccctggccctgggcagctactccagatggagccgagttgcggtgagagcaagagggacacagtgagagtgactcagagcctaggacacctccagaaggcttttcaaagcttcccgagtgtgggcacattaaaatagcaagttggacacatccagatggaatcccttgaagggtagcgtttcttgggtgtgttctatgttgaaaggctttcttcctgctctctaatatattccaactgtctacatgcaaagctaccatttaaaaggccatgcaatgcagttctgggaaggtgcagccaggtacccctgcattctttggttccatgggcttgcccctgagagcatggtttagcatagagacttagatgtgggttcttcattgaggtgggtggtgtgtgagcaccaatgatgcctgcccactcctcagccaccctggagagtacaaagggtctgggcaggtgtcctggtagccagccctcctcactgaattgcaggtggattccaaggctactctgggcctaggtggggtggctgttgtgctgggagcagtcgtggctgccatgggcttctactcctacctgggtgtcccctcctctctggtcatcattcaagtggtacctttcctggtgctggctgtgggagctgacaacatcttcatctttgttcttgagtaccaggtaagaagggaggggttcttcatactcaacatcctcattagacaaagttctgcacagactcactggaattctggtcaatttatacgtgtaggaaatagcctgggttggcacaaattcattcacactcattgagccatcttgaacttgcttccagttaaacccatacagcatccagtaagctttgtaatggattagaggtacctctttcctgcctttacattaccaggggcggcattccatggtataggcacaagccagagtccagatagtctctctttgctgtcaaacacttggcgtgacatgaacacttggtcgtttccacatctagaacgcaccagtggttctttacatcccaacatagaagcagagagcgtggctgtgagctgttagtaggctcttctgtccacggaaggtctggaagttcctcagatttggccaggaatccaaaccctaaccaccccaatgctgacctctaaagtttggtgaccttgggctggagaaatggctcagcagttaagagcactgactgctcttccaaaggttctgagttcaattcccagtaactacatggtggctcacaaccatctgtaaagggatgtgatgccctcttctggtgtatatggaaacagctacagtttactcatatacataaagtttggtgaccttggcacacccgtgtactctgtctctttgcccatgcagaggctgcctaggatgcccggggagcagcgagaggctcacattggccgcaccctgggtagtgtggcccccagcatgctgctgtgcagcctctctgaggccatctgcttctttctaggtgagcaagggctgtccttctccacccgggatgggatttgctaggttattctaagagggagcccaggctttcagaaggcagtgggtgttccctgctttaagctgtctgtgctggcatgtggcccatgatgccagaatgcccgacagaccctgtgccctcgacaggggccctgacctccatgccagctgtgaggacctttgccttgacctctggcttagcaatcatctttgacttcctgctccagatgacagcctttgtggccctgctctccctggatagcaagaggcaggaggtaagttcaactgggccaggacaagggacttaccctgccagtgtccctatattctctggaagatgtggcacagaggtagccagaagagtttgatgggaggcagggacagtattctgagagagaatgtttggggctctgtgctcaccaatttcctgtaaaaagagaatttctttttagttatttgtggtaacatcatcaacgcccctaaaagtatgtaaagtttacaaaataaattgtaaataaaaagttaacataaattttttgatgacggaaaattcagtatttgattaagacaggaagtaagctgggtgtggtggcccatgcctttaatcccagcacttgggaagcagaggcaggcggatctctgagttcgaggccagcctggcctataaagtgagttacaggacagccaaggctacacagaggaaccctgtcttgaaacaaacaaacaaacaaacaaacaaacaaacaaaaaccaaaaagacaggaagtaaaagcaacaaaaaactgcgtgggggctgtagagacggctcagtggttaagagcactggctgttcttccagagatcccgagttcaattcccagcaactacatggtggctcaccatccatactgggatctgatgccctcttctggcagcaggtgtccatacagttagaccactcatacaaaatcttctgggcttccttgaatgaggtggatcctgtagtctgccttggacccagtcttgagggcctgtcattctctaggcctctcgccccgacgtcgtgtgctgcttttcaagccgaaatctgcccccaccgaaacaaaaagaaggcctcttactttgcttcttccgcaagatatacactcccttcctgctgcacagattcatccgccctgttgtggtacgtgggctgaagggctgttccacttttgtaccactttgggagggaaaccgggcagagcatggtggcatgggaggctgcccaggcccggagcagacacttggagctagagcttgagcctgtccaactctaggacgtttcccaggatgcccaacaaagccattcaaatttgagggaagatgaaggctgtttggggagaggttctcacgtgccagtttttccctcagctgctgctctttctggtcctgtttggagcaaacctctacttaatgtgcaacatcagcgtggggctggaccaggatctggctctgcccaaggtgagcctggccttttctcagccctttgtcctgggaggggcagcagtgcccaataggtggagcggtggtggtggtggtggtggtggtggtggtggagcttgagagggggacatagcacaaggcttagccccatgcagagttgctctaagtggaccgtgagagagaaagcacatccatgttgtaagtgtgagcgctgagtgctggctcagggtcacagtagatgtcctgtgctggaggcctatccacatggccattcacacagggtggggcgccacttccttctatgtcagttcctcaccaatagctggtttcggatttattactttatctgtacgagtgttttgtctgcgtgtatgtttttgtgccatgtgggtgcctggtgcctgcctgcagaagtcaaaaggagggtgtcagatcccccgggactggaattacagatggctgtgagccaccctgtgggtgctgggaactgaacccgggcattctgccgagccaactctccaacctcagcacttgttatttttctgtgttttttttttttttttttttttttttttgtgtaggggaatcaaatctgggatctcccatttgtcttgtttcgatctcttgagagtcctagcaacaccgctgtctggctttatagtttcgatttgcattttctttctttttctttttaaagatttatttatttattatatgtaagtacactgtagctgatttcagacaccccagaagagggcatcagatctcattacaggtggttgtgagccaccatgtggatgctggaatttgaactcagaacctttggaagagcagccagtgctcttaactgctgagccatctctccaggcccctcaatttacattttcaacaattagaaatgttacataccttttcatgtacatgttgatcactatatatcttatttaagaagaaatgtgctgactttgctcggtttttgaattggctttttgttgttgctgagccttggagagttccctgtggattctggaggttggtgtcttctcagagacctgattatcaaatggttttctttttctgtgggctgctctgttattctagtggtggtgtgccttggtatgccaaatatttaagcatatccatggattctttttctcttttattgtctgaatttgatggcatattaaagacataattgataaacagaaagttattaagtttgtctggtttctattaaggttttttatgactttagaacttctgtttaagtctttgattcatttgagatttgctcatttgttttttgaaatagggtttgtctgtgtagccctagctgctctggagctcactctgtagatcaggctggccttgagttcagagatccacctgcctctgcctccaagtgctgggattataggtgtgtgccactaccccactttgaattgacctttatatatgatgttatgaaagtggacaaattttaattccatccagctttcccaggactgtactaagaagtacagctctcctccatccgatggtttggcagccctgccagaggtcattcaagcatgtctgtgcatgactctttattctgctccattgaaatttcatgccggcttccgtgtcagcagggccctgctttgattcatacggagttgcaaaccagaaaatgtgagacttgcaaatttgttctttgtcgatttctcttgggctatttgagttcttgtgagattacacttgaattttagcttgacatttttagattccttcaaaaaccatccttggcatttatgcagggattgcactgaatctgcagatggctttgccttgatagtactaataccgtcacaatatttgtcatccagcccatggacacacgatgtatttttttttcatttttttctttaatttcttctaagaaccacatctccaaatttttaattttttttttttgagacagggtctcactacgtagccctgcctcactgtgtactcacatgtagatcagactggccttgaacccacagacatcgcctggctctgcctctcaagtcctgggaccaaaggtgtgtgccaccacactaggtctgagccactggctttccacatgctaagcatgcactcttaccactcagatgcaccctgagccctcctctctgaaggatagttttgctgtaaataaggttttttcttccctttagtactttgaatacatgaaccgcagtctccaacggcagatgggaaaggtggaagcagctgcgttagtcctttgtgacaagccattttttgtgtgttgtccccagagctctctgaggttggcttttgacagctgtactacagcctgccttggtcaaggtttgagcttgtccttttagacgtcccaggaattcctttaaggttgatattcgtgcctctctttcatcgatttgggggagttttggctactgcttcttcaaagatcacatccagattctttcatttttcttccttttctgattttgtttttgtttttgttttttgtttttcgagacagggtttctctgtatagccctggctggaaactcactttgtagaccaggctggccttgaactcagaaatctgcctgcctctgcctcccgagtgctggaattaaaggcgtgtgccaccaggcctggcttttttctgaaattcttacagtgcatctgtgggcctattggcatcgcctggggccctctctgtgtgtatcttggtgtgcccttttatgtttgtatctgggcatgttcctcgaagtacatagtgctgtgtgtacctcagcctgtatcttgtatatatgtacacatgtagtctgtacatttctgagtaggtttctgagcatgtgtctctgagtgttctgagcatgcatctctgagtgttctgagcatgtgtctctgagtgttctgagcacgtgtatctgagtgcgtccctgagtctgcctccgagcatctcatctacctcgtgtacctctgagtgtgtcttctgcttccagatacatctgcatggacttctgagactgtgctctgacctggggcggacccactgtggatatttcctacactcacagatcctcttctttcccaggattcctacctgatagactacttcctctttctgaaccggtacttggaagtggggcctccagtgtactttgacaccacctcaggctacaacttttccaccgaggcaggcatgaacgccatttgctctagtgcaggctgtgagagcttctccctaacccagaaaatccagtatgccagtgaattccctaatcagtaagtggttggtctccccgacaccctggcttgttccttctctgctttctctctccattcctcttctctcttcctgcatgctctgtttctgcagctaacaaagccaggggaggctccagtgcaagggtaaggaaggagtccccagcagactcattggctccacctcctcctctccactgtctggcctcaggtcttatgtggctattgctgcatcctcctgggtagatgacttcatcgactggctgaccccatcctcctcctgctgccgcatttatacccgtggcccccataaagatgagttctgtccctcaacggatagtaagtttggggctacaggaggctcactgcccattacagcttagggaaactgaggcaggagaaaagaaaggctctcagtctcccatcaaacccatagggtccaggtggtttaggggttaggcactcacactatcagtgtcccctggagtattacacctttgtttgcagaacatgttggttgtgggcagtgggctatggagttggaagtggagctatggccctgcatatggagctgctgtgtttaacaagtgtgggagatcccatttcttgaccccacaactgggggtggcaggtgtaaacctcttagaactggggactttagatttgggcacagaatgggagtcaggacaggagctgccttgcctggtgtgtcactgcccagagtcctccctctctgcagcttccttcaactgtctcaaaaactgcatgaaccgcactctgggtcccgtgagacccacaacagaacagtttcataagtacctgccctggttcctgaatgatacgcccaacatcagatgtcctaaagggtaggttccgagggtggctcttgctggagactggggagactagtgggttctagaaatggtagacacagaggaggcaagagtgcctagccaagccctttctggggcacagtgagtggactgacaggacaaggtctcgttccctctaagcctctactctgtcctccactttgcaggggcctagcagcgtatagaacctctgtgaatttgagctcagatggccagattataggtaagtgtgatatggtttggggaggagatctcaagtcagtcagctgttttagagtcctctaagagcacccatgcatgtggctgacgtgtgtgtgtgtgtgtgtgtgtgtgtgtgtgtgtgtgtgtgtgtgtaagttagcagggtgtgagggtaggtgtataactgtcctgggcctgtaagcaccggttctccttctcaactccacgagctaatacttcacccattctttcaccccaagtccattgccactgtgaaatgtgtggcagcctttgtaacagctgacgtcattattgagagccacccacttcaacacatattcactctcgtgtgtatcatatgtccatgtcagcagcaacttctggctaaatgaagtaggatgcctttgtgtagtggaatctcaagaggcatcaacagtaactagggagtgactctgacaggtggggaggagcttaatccagcaggcagcagaaccaaccaatcatctgcatggagggagccagctgttgttagatctgtgcacaccactatgagtgcaggagctggcaagacgggtctgggtgctttggatgaattcagttttttacctaaataactccaagttggaatcactagctgtaaactgatggcagcacatctagtccaccctctaagctaactcttctatgaagttcctatcttccaagaagtcacaccaccttaccctaggcaacagaggcatgtggtcaaaagggatcggggtatggcagacagaggaatggattgtttcttggagggcaggtctgcatctgtcatggccctaagtatcccacagcagcgcttttctttcttttttaatttttaatttttttggtttttcgagacagggtttctctgtgtagccctggctgtcctggaactcactctgtagaccaggctggcctcgaactcagaaatccatcagcctctgcctcccaagtgctgggattaaaggcatgcgccaccactgcccggcagcagcacttttcaaaatgagagttcccctctctcctctaacagtatcagcattatcaggagatggctagccatgcccaccccttgcctagccatgcccaccccttgcctgagccatgccagaccatatctcggcacgaggatgaccatccttctgggtgtgagcaactagtaccttaagattacgcattggcgactatttctcatctgtctttgttttccctgtgtgtgcctagccctcctttctttagcggatgcaacactgctgaacacaattcacagttgtttattgtacttacaggcaggatcccaaagtcacagctttaataattcagtcatttttgtttgcctgtcttcctttctagtctgttcccacaggaataggcaaactgaaaattaatttttttgagacagagtctcatgtagcccacactagtctagaactttctatgtagtgctgaactcctgaatctccagcctccccaagtactgggaccacaaatatgaaacaacacactccaacaagaaagtaggctcaatttttgattaaaatccagtgcatattctgaaagcacacatagaaggatttggtttcaccagggagatattttagtactttagtttagttttttttttttcttttttctttttttgagtttctctgtgtagccctggctgtcctggaactcactctgcctcgaattcagaaatccgcctgcctctgcctcccaagtgctggcattaaaggcgtgcgtcaccactgcccggagagattttagtttttgctttgcttttgaaacagtatcttactctctagcccaagctggccttgaatttgaagtaatttccctacctgtctgctggctgctggaatttcaggtggtacataaggcatgatcattcaagcttcatctatcaaagtcaggccttggtctcagtggcagaagtagagtttgaggatgttgatgcaaaatgtaaatgcaatccttgaggctggagagatggctcagaggttccagaggacccagctctgactcctgcactggtgcagcagatcatgactgcctttaagtccagttacaggcgatcccatgccctcttctggcctctatgggcaccaggaatgcatgtggtacacagacatacatgcaggcaaaacactcatacatgttaaaaataaataaggaaatgcaggtctcttgtagagaatcgatcaggaatttcaagtgctgctggtggcaccctgaacccaatgagatcacatctgtgttaatttctccgggatacgccaatgggcactgatctttgactttcagcctcccagttcatggcctaccacaagcccttacggaactcacaggactttacagaagctctccgggcatcccggttgctagcagccaacatcacagctgaactacggaaggtgcctgggacagatcccaactttgaggtcttcccttacacgtgagaactagagggctgagtcaggggtgtgggaggaaacagccacacaagagatgttggggggactgtaggctttgagtgatcctgtgggatgaggaccaactttgtctcagctgtctcctggggtagcttgcggcctgtccatttcttacacaggtcagcacccaactcaaatctgggtcccttgttttccttgggagatgggatgctcatatcacctgaagggaggattgatgctttgaccacaccctgatctttggcaggatctccaatgtgttctaccagcaatacctgacggttctccctgagggaatcttcactcttgctctctgcttcgtgcccacctttgtggtctgctacctcctactgggcctggacatacgctcaggcatcctcaacctgctctccatcattatgatcctcgtggacaccatcggcctcatggctgtgtggggtatcagctacaatgctgtgtccctcatcaaccttgtcacggtaacccacagagcgggccttggaagttgacgatctacactcataagctaccctcattctatgtagaatctagacatccggctggcttgctttcttgtacccacaccccaccttctcccttgtttcctgaagttcatttctcttgcctgtaggcagtgggcatgtctgtggagttcgtgtcccacattacccggtcctttgctgtaagcaccaagcctacccggctggagagagccaaagatgctactatcttcatgggcagtgcggtgagtggggagggatggcctcaccctgcgatccacctgagcctttatgtcctcctgtgctgactcctggctgtgactcctgccaggtgtttgctggagtggccatgaccaacttcccgggcatcctcatcctgggctttgctcaggcccagcttatccagattttcttcttccgcctcaacctcctgatcaccttgctgggtctgctacacggcctggtcttcctgcccgttgtcctcagctatctgggtgagtacctgtgcacacccggccaagatgtcacaactgtgagcattgatcaaaatggtgcctgctcccctggaaaacttagagatttcaggctgagggttttaccatacatcctactttgggagcttttgttttacatttaatatgcagcaagcatctttctctgtgagttgattgtgtcttaaacactgtgtgggctgctgaacttttctgatagacgtttatgcacatacaaacacacacacaaatggacacacatgcacataaacacacatactcacatagacacacactcagacacacatgtacacattcacacagacacacatagacacacatatatacagagacacacagacacaagcatacactcatacacacagacacacatatagacactcacagacacactcacacaagcacagatacaaatacacacacacactcacaatttgaatgcacagacacacagatacagatacatacactcatactcacatataaacactcacacacagacacacaaaaacatgtacaggggctggagagatggctcagtggttaaaagcagtagctgcttgctcttccagaggtcctgagttcaattcccagcaatcaaatggtggctcacaactatctataatggtaaccaatgccctcttcaggtgtgtctaaagacagctacagtgtacttatataaatgaaataaataaatctttaaacacacacatacaaagacacatactcatatacacaaacacacacacacacatacagacacacgttcacagaagcacagacacacactcacagatacacacagacatgtacataaaagacacacacatacacagacacacacacacacacacacacacacacacacacacttctctgtagatggaacccagaatattgcacatgctaggcaagtactgtaccactgagtcacaccttagcacagaatacatattttacaatgagaattgatgagtgaggtccatgagattgtctgagtagattctgagcctcttgctcatatagtaagagaaggtgtattttggccaatcacagtatatatgtttggaatctcagggtcctcatggcatcaactgtagtcactgaggctctgttttggaccaattacagctttcctcttggcatcaatttcatcctggattgtgctttaggccaatcacagtcttgcctcttagcctcacaacagtctccagctacatcaccaggggtggtggtggtggtattcaacctacagctgatgaaggcctaagggccgtctggctgatagttcttcagggcagacagaacagcaaggccgagtcccacaggtggtgtttaggaaagcagattgccccatcctgcagcatcttagcttgcttacagggacatgggcatcaggagcgcttacaatataatctaaagagattattaggacgaataatgatcttaatatattgttaatggtgctgcacatgcttaactcaccaagtaccccaggaagaagtgtttcccttatctgcatcacttcctccactctctatttaagcagcctacaacttctggtcattagactatttctgatgctatactactgttgctagcactacagtaactgaccttgttgctgaatctttgaccttgtcatccagtattttcttagagtaaacctggagatgttgattattgatgtcggttgttaatactccccacaaagttcaatggtgatgataatggtggtggtggtggtggcattagagtcacctacaggaactcactgactatctttgtggagaagaatgtgtatgttggggacagtgagggaacagccctgggagatgttgccagcccagagcctcagagacacaggctggaagtttctagacctatatggggtggagagtactgaggactgcaagcctcccatccccagtgatgaagctgtagtcaagaataccctgaagctagggctatgcaagcagagtcccgaagggcatgtggtgagtatagagccctactccctggttgccttgtgccttgggtttgtagttacaatataaggtatgctttttagaggagagttgaccatggtgtcagtaccagattgtcccaggaacaaagggagagagagggtgtcagggatcctgttgagagagcatgccagctgaggcaggctggtgagggtggtggaaataccagctgaaacagctgttggtaatgtgaggcaagcgcaagcaggtagagccgggctctatctagatttgcataccgctgtgatacctgcgtcatctgtatgcctctcagaccatagatgtatgttctttctcttccacagggccagatgttaaccaagctctggtactggaggagaaactagccactgaggcagccatggtctcagagccttcttgcccacagtaccccttcccggctgatgcaaacaccagtgactatgttaactacggctttaatccagaatttatccctgaaattaatgctgctagcagctctctgcccaaaagtgaccaaaagttctaatggagtaggagcttgtccaggctccatggttcttgctgataaggggccacgagggtcttccctctggttgtttccaaggcctggggaaagttgttccagaaaaaaattgctggcattcttgtcctgaggcagccagcactggccactttgttgtcataggtccccgaggccatgatcagattacctcctctgtaaagagaatatcttgagtattgtatgggatgtatcacatgtcaattaaaaaggccatggcctatggcttaggcaggaaatagggtgtggaacatccaggagaagaaaggattctgggataaaggacacttgggaacgtgtggcagtggtacctgagcacaggtaattagccatgtggcgaaatgtagattaatataaatgcatatctaagttatgattctagtctagctatatggccaaggtatttataaatatatttcgagtctgagtcttatttctgggagcatggggctgggtgggaagaacagggcccaacaatcctccttcttgcccagggtcttgtagttgccgggaacatgtttgtatctctcacccagcatttcctccccttatcaaaactatttccagggctggagcacttgttcttagagagaacatgggttcagttctcagtggttcacaatcatctacaattccaatgtcaggaaatttgacaccttctgatgttcacagacaccaggcatgtggtgcacatatgtacaggcaagacactgatacacacaaaacaaacaaatacatctaaaaatgatttaaagaaaacatctttagggccagtgagatggctcagtggttaaaaggtgattggcatcaatcttgagtttgaccccctggaactcatatgatgggaggagggaaccaactcttggaagggctcctcttacctctacatccatgcattggcacccctagcccccagaaggtaacaacatattaataaagtctccgttctaggatggggctgtagctcagtgctggagcagcagcatggcagcctcatgtacatgcagtgtctgtcacctgccatcctcagtactgaaaaggacagagagcaagagccccgaccttgtccctagatgttaccacttccagtgacaataactgcctttgtttaccactgtccctgagtacatttaaaaaaaaaccctccattccatatcagcatgactgttaaatgactgttaatatttacctatagccctaggacagagtgtgacccaccctgggctgtaatgttttagaagagcagggaaggcaaaggggacctaatgtcttcctggcttgaggaggtcacagtacgctgggagtggttgacctcatctggaaaatggcattcagtttggcctccagtttcctcagctacagagcatgttgcaggcgctgtgtgtctgtctgaaggcagacagctctgggctgggcaggttttctggcatgggtcttatggctggagcacaacctgaatctggtgccttggttgcaacagagacagagaagaagataccttgtttgtgaagcacagactttgttgaatagtgtcgtagagagtgttttactgctgtgagcagacaccatagccaagacaactcctagtgtcttagagagggttttactgctgtgagcagacaccatagccaaggcaactcctagtgtcttagagagggttttactgctgtgagcagacaccatagccaaggcaactcctagtgtcttagagagggttttactgctgtgagcagacaccatagccaaggcaactcctagtgtcatagagagggttttactgctgtgagtagacaccatgaccaaggcaactcctagtgttgtagagagagttttactgctgtgagcagacaccatgaccaaggcaactcctagtgtcttagagagggttttactgctgtgagcagacaccatgaccaaggcaactcctagtgtcttagagagggttttactgctgtgagcagacaccatgaccaaggcaactcctagtgtcttagagagggttttactgctgtgagcagacaccatgaccaaggcaactcctttaaggacaacatctaattgggtttggcttacaggttcagaagttcagtccattatcatcaaggtgggaacatggcaaaatccaggcaggcatggtgcaggaggagctgagggttctacatcttcatctgaaggttgctagaagactggcttccaggcagctagaatgagggtcttaggctcacatctacagtgacacacctactccagcaaggccacgccctctacccccccaccctctcccgggcaggatacattcttggagctggaagtttactgaatgggtcccttgatcttgaatgcactccctgggccaagcatatgcaaagaaaaatgatgcttttatcacgtgtcctgctctggctgcctctgggttaaggataacttttgtacaggatacaatcacaatgacatgcacatcagggacatttatggaaatattgtttttgttctattcctttccattttcaagaggatagtcttgtgacatgttcaactgcctatgagagcctgtgatgggcaggggtactgtcctccactgtgaggcacagggttagaacatggggcctgtgaggcgggcacatttggagaatctactggagaatgccagagtgcattcaagatcaagggacccattcagtaaacgtccagctccaagaatgtattctgcccggtgtgtgtgtgtgtgtgtgtgtgtgtgtgtgtgagagagagagagagagagagagagagagagagagagagagagagagagtgatgggggacattccagatcccctttgagcctcttcactctctctgtccagctcctgtttggctgaagagcagctgtggttcctcctgtggaaggaggaaaggagctagcatctctggaaactgctgcctactttctagtctgccagccccctgtgtattattactagctggtctttacaagggtccctggaaggtcaggaagtatgtgaaatctggttaaagaagctctgcctcagaacagagggtgaatctcagctattccatgttaaatgacaccttgtcattagctgtcctgtgcatgttactggggaggaaagtgtttctcagtcctagccagctgtaaacctggcaagatatgtacacaggcgtaagggagacatgaatgttatggggccaaccaaccattttctaatttttgtaaacaagtcttttaatttgtttatttagtttgtaaatgtgtatgcacactcaacttgcatttatgtttttgcactgtgtgtatccctggtgcccgtgaatgccagacaaaaagtgtcaggtctcttggaactggagtacaggctgttgtgaaccaccatgtgaatttctcttcaaggaacagcaatgttcttttttttttttttttttaagatttatttattacatgtaagtacactgtagctgtcttcagacacaccagaagagggcgtcagatctcgttacagatggttgtgagccaccatgtggttgctgggatttgaactctggaccttcggaagagcagtcgggtgctcttacccactgagccatctcaccagcccaacagcaatgttcttgactgctgaaccttttctccagcctcccacaaaccactttctggttggacttgaagccagctccacaaggtagaacccatgcctggtaccattaacggggctaaaaactgtggctaactacattataggccatggggagaacctagtcttattatgttaaatggacatagtaaaagacttcccttcaagatctcatcttcatactcagagatcagttcattcctcaacttttatcagagaaccgtctttttgagtaggtggtgattgatacagcaactggtcaccatgcagagactaagactgtggacagctcagctctaaataggatgtctatgtcatgtgtcctccccacaaggctcagagagggaagagagggcagaaagtctgtaagagacagagggagtgaaccaatgcagtgagactgtgtttgccagacacgataggaccattgcatatgtgaactcacaggggctgggaaggcatgtacaagacctgcttgcctaagatcaagccaaccacaaccatagcaagataagtgagggcccaagaagtcccaccccatctgaggcactactgacagctgagggctacataatctcacccttctccagggatgcaggctgtggtggtttgactaggatcagctcccacagactcatgtgtttgaatgctttgctcataaggagtggcactattaggaggtgtggccttgtcggagtaggtgtggttttgaggtctttgcttaagccatacctagtgtggctcacagtcacttcagctgcctcttgatcaagatggagaactctcagctccttctccagaaccatgcctgcatgcatgctgccatgcttcccaccatgacaataatagactaaacctctgaaatgacaagtttggctccaattaaatgttttccttataagagttgctgtggttatgatgtttccttaaagcaatagaaacccacattaagagacggtccctgagaggctacccatgttccagtaaacaggtccacactgaatggatgaatggacagcaatgaatggactcagtgggcatcaaattgaaaagaaagggggtggggtagaaaacatttgtacatgaagttgggagggaaaaattgtgtggagctagggaggatctggaagggagaggatagggggtagattgaatccaaacacactatataatatttacgaatcataaaactaaacaacctcaagaagaacctaggggagcctgtataatctgggagggacagtttccccaagcatagatccagaagccgtaaactaaaagcaagggggccctggggaagtggggaagggaaaagacccacaccccgccagagttccacctactctctggtcagtcaggtgtgggaggggtgggcattcctctatcccactctttagggagtggccaggggcagccctacctggggaccctggagctactttgctaaagccaccagggttataggagagagggatgagggaagagattcccaacacctgtgagagtacatgcagccttgatggagcagagactctctatggtttaagagctttattatagaaaggcagggagagaggggggggctagaaagagtaagagggagagaggagagaagtcaaagagagaggagagaggagaagacaaagagagggtgagagagagggtgagagtgaggggtaagaagaagacaagtaagaggagtaagagagcgaggtggggctgaacagccctttttatggtcttcactgttgctaggtaactggggaggagtttagtctgaaggtcagaagcttgggccattgcctaagtgactactgaccatgcttctcttgttggggctgtgggggacagtagcttaggcaggagccagagttccaggagcataagggaacgcctaccgtgtcatgaaggtgaattatgactttggggttcagaactcagcttaactggagaccagcctatctttgtatagcccaatgccccacgcatattcaaataggaaccctctgtaatacaaaccaaaataaacaaaccaaaatccaaaagtaagctggaaaatgcatagtggtctgaaaaaacgtttgccctacgttcaacatacgaggactaaaatcaagaatacacaaagagacaaattagtgcagtgagtatgtggataactgtcccatgggaaacaacatctctggctaaaggaggctggggaggttgctcagttactaaagtgcttcctgttcaaacatgaggagctgagttcagatcctcagcatccatggaaaaagcctgtgtgacagcatatgcttatgatcccagtgctccagaggcagaggaagaggatcccagggcttgctctcattcagtggagccaaatccaagtgcagttgaaagacctgccccctactccccaacaaaccaaagccaaaacaaaataaaaaccaagctgggcagtggtggcacatgcttttaatcccaacacttgggaggctgaagcaggcggatctctgagtttgaggccatcctggtctacagagtgagttccaggacagccagggctacacagagaaacgctgtctccaaaccaaaccaaccaactaaccaaccaaccaaccaaccaaccaaccaaccaaccaaccaaccagtggagaataattgagagagacacctgatgtgacttctggcctccatctgtatgtgggaatgcacagtatcacaaatgtatttatttaacacacacacacatacacacacacacacagcagttaataaaaaagataagctccccttttactgttgcttgatagctcattccttcttgttgctaaatagtatttcctttcatgaaccattctcttggcaaactcttggctgcttctaatttttgcagttatgaggaaggcaatgaaggtttctttgtaggtttttgtatgatcacagttttcaaatgctgggcaaatatatggtagcatgtttgctatgctgtaaagttacctttagcttctcagtttttttaggtccttccttctttcttttcctttctctttccttccttccttccttccttccttccttccttccttcctttcttccttcctggtggtaagtgggactcactttgtagctcaggcttgtcttgatactcttcctgtctcagccttccaagtgctggaactttaaccataagccaccccaccagactactaactattatttattggtttgtttaattattgctttttttcttttcttttagacaaataactcactgtatttagcctcagtgggcctgccacttgctgtatagacctggctggccttgaactcacagaaatcagcctgtctctgcctcctaaatactagagttaaacctgtgtgccaccattcccagcttccactaattttatttactttttttttttttttttttccgagacagggtttctctgtgtagccctggctgtcctggaactcactttgtagaccaggctggcctctgcctcccaagtgctgggattaaaggcttgtgccacccctgcccggcactttatttactttttgagtgtgtaattaatgcaaatgcattcagtagtaccctttccttctattttgtaccctatttctccttccttgatgtccctattccagaggcagactctgttctttctctcttttttgtttttgttggtttgttttggagagagggtgtcatgtgattcagtctggccttaaagtctctgtgtagctactgttggcattcacgttctaatccttctgcctctgcatacaaatgctagcatgccaggtgtgtaccactatggctgatttctgctctcttccctgtgataccgtgtagatagtaaagaattattcaaagtggctgggaagattcctcggtggttaaagcacttgccatgcaagtgtgaggactagaacttggatccccaagaaccaatcaatgatcaatgggcgtggttgcctatacttccagcctcagcagagaaagccggctggcatgaccagctaaagcagcgaactttgtatttgactgagagacccttcctcaatgaatggtagaagagtgggcaaaggtgattcctgacgttagcaagtgctcttcaccagaaagccttttctttagcattcacattatttctttttaaaagtctgttgacagcaagcagcactgattcagtgaattacataaaaaaagtaaatgaggtcgaaggggctcatgttgggggtttggaatgtaggaattgtggttcatatggtcaagatacatcgtatatatgcattaaattgtgaaaaaatattcattttatatttttggttgtgagcctagcctttaatggctgagccatctctccagcccaaagatattcttttttttttttgtttttttttgtttttttgagacagaagatattcttttttaaaatatgttgcctgttgaggcctgctccttttaatatagcagtagccattttgtattctgtctccattttgctcctaaggtgaaatgaagttcaggttctcagactctgcttcccagaagtgagcatccagagctgacactaagtatgttactaataagccaaaaagttacggccgaatcacttgtccctgttatctcaatgttctgaaattccctgctcagtacctgtccaccacccttcttacctcagtcaggaccactcagcttacaggttggctaataatactttatctagttagacaaaactgctgtaccacttcactgcttgcctttgaacctttttttaagatatatttattatttatatgtaagtacactgtagctgtcttcagacgcaccagaaaagggtgtcaaatcttattacggatggttgtaagccaccatgtggttgctgggatttgaactcaggaccttccgaagagcagtcagtgctcttaaccgctgagccatctctccagccccatctttgaacttttgaacctggtttttcctataaaaagcctgccctgaggaccggctggtgccacagttaggtttttccttcttgtggacctagatgtccagtattatgctgtgtgttcaataaactattcctgtttaactgaaattggtgtacgtatggtttgtggcaagtctcagaccccgacactgacatgtgatgtgtatggttatttgcaaattaataaatttaagcattaactttcagtatagtaaataatgatgaataaaacataaaaactgtttggattgtcaacaaaattttctcatcgtctgtgtatgggtgttttgcctctaggcatgtatgtacttcatatgtgcatgcagtgtcctctaacgtcagaagagggtagcagattccctgggtttatagatgattgtgagccaccatgtgggtgctgggaatcaaatctgggtcctctggaatatcagctagtgttctttgtttttgtttttctgagacaggatttctccatgtagccctggctgtcctgaactctcttggtagaccaggctggccttgaattcatagagatctgcctacctcactggcattaaaggtgtgtgccaccaccaactgtctgagccatcgcggttgctccatgggttcttgaaacaaaatttaagatcataattttttgtttggttggttttttttgacacagggtttctctgtgtagctctggccgtcctggaacttactctgtagattaggctggcctcgaactcagaaatccgcctgcctctgccttccaagagcacgatcataaattctaagttgaaaaaatttacatcaatttatctgtatggctttacttaaaatttgctaaggcccaacactattaagttatttgttaagcctggaatgtctcttactggaaaagcattttcctaacatgttcaagaccctgagtttgtctcctagaactacaagaaaacaaaagtaaaatcagtattttgctctgtgtggtggcatatatctttacttctgcacttggcaggcagaaacaggcatatttctgtgaatttgaggacagtttggtctatacagcgagttccagggcagccaaggctgcagagtaagacaatgtcttttaaaaaagttaatttgtagatgttagttagtctagtagagatgaaattcattaaaatcttttttgttgttgttgttgttttttttccggtcaggatctccttgcagagcccaagttggtcttgaactggctatgtggatgaataaacatttctggttctgtcgccaccttcccagtgctaggtttacaggtatgggttactacacagtttatacaacattcaggacacattagtcacacatgtgggttactacacagtttatacaacactcaggacacattagtcacacactttaccaatttagctacattgaatgaaaaaaaaacaaaaaggaggacatgatggcttctaggtatggtggaggaggtttattgtagacaggagggagcagacagccagaagcagaggcatctgggagaattcagggtggaagtggctgtagaatgagctgggccatgtgagaagggttaggggagagggtagaagagacctggagtcaagaggccaggagaccaagaggccaaagggtaaaaaggacctcataaccaaaatggctgggttacataggaatcagagaagcttggggaggaaaaggccagctcagactctggactggagaagtttagggtagaggtcaggattagtatgccagccagaaggatcctgtaccagaaggtactgagggagactggtggccagagtctgctttgatattttattaggcatctcagccatttgtcttcggtttgtgacctagcatatgttcctaatctgtctgtctttcgtctccccccaaccctcttctgagactgggtttgactctatagcccaggttggccatgatttggatgcggggattacaggtataaaccacagattgattgtgtgtcaaccttacataaacgttttcttaaaatgtctatatgcacgtattagtaacggcacgtgtatacacacatgctatacatacggatgcacacacacatatttacagtatcatacttctttttttccttcactgagcttttcacacttttcttgtcctttcaagtcaccttgaaatctggtgcaaccttctgagtttcactcatagctctgttaataggaattccatacaattctacaatattccttttccgttccctccgctaacaaacaaaatgtggtattataagaaggcgcaccagacacctacgcaggaattcaatccagaaagaagaaaggctcccagaccacgtgacacctccagggactacgtcaagtggccgtcaccacaatgcttccgccctcttcaaacatggttggcaagcgctctccgcatcgtgaccatggttattcttgcatgtaggaaccgtactgagcgcataccaatctcctttaggcaagtgtcgcggcggaggagatccagcagagccgcaagaacgacgatcggttaccgccggtagtaacaagcgcggaccggaagttccgcgtcttcgctgtccggggggagccgttaggcgcgcacgccggaagtggccaatcagccggtgtgaggcggtgcccactgtgttcgcgtccctcgggcagcagagccatggagcccggggctgctgagctttatgaccaggccctgttgggcatcctgcagcacgtgggcaatgtccaggactttctgcgcgtgctcttcggctttctctaccgcaagaccgacttctaccgcctgctgcgccacccttcggaccgcatgggcttcccgcccggggccgcacaggccctggtgctgcaggtgaggtggagagaggcggcgggccgttggggtccagcaggtccttaccccagttccacctcccagcgccagaggtgccccggctcgcgtcctacgtctgggaactgcgccactccgtagcccacccttcagggtttgtgattctctctggggtgccaccaggtgatttgagtaaatggccaggcgttatctgacccacggatccgttggcaggaatgcgcttctttgggtacaggctgggtttgtgcgggagatgcttaggtgttggacctgtcagcctgggtttgagggcctcccagcgccctgtgggcatccccagaacggtgcaaggggcttgttaggcagggttcaaaccgtgcaggctgtaggaggaggactttcacagcgggagaaactagtagatttcagaattcccgggctcggaggtggcaggaatgactggtatgtattgattggtgggtgtggcctcttgccagtgacttactaagttggtggacaattgtaatgtttacgaaatttacatttggattaaaatttatatgtcctaggttatatttattgtttttgaagcgttactaatgactgactcaagttgtttggacacctttttaaaaactgttttctttcgagacaagatcttgctgtgtagccctggttgacttggaacttgccaggtagatcaggatggcttcgaacttatggtcctccttcccgacgctcccgcgctgggattgttgacattgttgagttagtaataagcaaacatttaatgaatatatttaacactctcagctgcggtatagactctgatggtatggatatttaaataatggtctgtctacttttgacactgtacagagagtaaagcacagacactaatagagagctctgctagttctctgtgtggtagagctctttggaaggaagtgatagaagtggtagttaggaaattgagacagttttgtaggagacgttgaagtaggttttgaggggcgtgtagagtctgattcacaaacctataaagtgcttcatgtcacctttgctgtttgttgacccctgcatcagccccagacagggctttcctgtgtagctctgaagtcctggaactcactctggcatcaacctcagatatctgcctaggattaaagactcgaattgccaccacctggcttaccttccatcttaatctgttctgtgggcttgtcaggggttactttcttcagttcctctcagtggaagaagccaaagcttagaatgttctagtaactcttaacataatgaccatgaggtgatcagtgggaaggtttgctactgagtttcagtttcagaatggaaaccgatgagtccctggggtagctcatccttatcagtggcccaagtgctgctggttgagtaattggagagggtgagaggtggcaccttgtgtttctttataatgaagtcttgccacctgatcgccatgctctctgaatctgacgtagtagttcaacaaggttagatgacaacaagaacttcactctgtttcctgcaactcaggacttcagttttctaattccaaatctttttgccctttttgtcatttagccactccatagtatggtgagaacttttgttttggttatgaaaggaggaaagagtatcccagtggctggctggcatttgaatttcttctgtataacatcttattttagctttaacacaatttgagaggttggttcttgtttgtctgatgaactgataaaggcaagatagatgcacttatcgtacaactttataaaacagctacctctgaaaggttaagatagctctataggttacagtgtggtgcccatgctatgggaggtagggagccgaggaagggacagggtcaaccaaatatttcaaggttccaaatctgggtaactggaagagtgaggtcattaactgtattgtcacttggatttggggggtgggaaagttttagataagttgaatttgagatcgcagacaggttctggcttcagagctgttggaatggtaggactggatcttgcaggagatggaaggactgtgaagtctgaagaagagcccagtgtagaggacagctggggaacggtcaccgtaaggggcattgggttctaccgcagccaaggttacagtcccaagtacaggtttaagagttgtttgttcttaccctttctccaaccccaggtggttctcaaccatcagccttgaccgagagcccttttatcttctcagtttctttcttttgtggggtgggaggttgaagacaaggtttctctgtgtagctttggctatcctgaaacttgctttgtagtccaagctggccttgaactcacagagatccgcttgcctctgccttctgagcgccggcattaaataaaggcatgtgccaccactgcctggctcagtttctattttcaaacaagtatttattgagtctccactataatatacactgatttgggccatgagaaacagaggcttataagttgttgtggggttttggattttttttttattctgagaaaaggtctcaccatgtagccttgactggcttggaacctactatgtagatcaggctggtctcgtattcaaagacatctgcctgcctctgcttcttgagtgctgggactaaaggcgtgcgccaccacatccagccaaccaagagactttttactatgtatacacttgaatactattttcatcaggtagatcatagtaagtcccccgaggatctgcttttttttttttttttttaatttactgaaagcctttgcaagaggcttgtaagctacatttagtattggtaagggttttggtatcttttctactcatttactgctttttctgttcttgattcagtgatcctaagactccttcctgtgacttcctctctcaaagtgtggttgctgcccagggtctgtgaacaggccggagtggtttgggtggtttctgtggcgcttgggagctccctaccgatgtcttggggagtagctgctgctgtgtgtgcttctttatgtcaactgaatccaaaccgagaccaaggcgtctagaaagacataatctggggctggtgagatggctcagtgggtaagagcacccgactgctcttccgaaggtccgaagttcaaatccctgcaaccacatggtggctcacaaccatccacaacaagatctgactccctcttctggagggtctgaagacagctacagtgtacttacatgtaataaataaataaataaacctttaaaaaaaaaaaagacataatctcagcccaggacttgccttcatcagatgattgatgtgggagggcccactgtgggcagtgccatccctgggcaggtggtatggggtgtataagaaagcaggctgcgtgagttagtaggtgtcattcctccgtagtctctgctttagttcttgcttcaagttcctgccttggcttcacctgatgatggactggatcctttaagccatgtaaaccctttcctccccaggttgtttgtgatcatggtgtgttttacacagccacagaaagcacacaagtgcagtgctgctgacagaggctggctgtctgtttcccttctcagggtttagagcatgagtgaagtgagtaaagattttgttctgttcattagtattagaaaatgtttttgttttgttttgtttttttttgttttttgtttttcgagacagggtttctctgtacagccctggctgtcctggaactcactctgtagaccaggctggcctcgaactcagaaatccacctgcccctgcccctgccccccccccccccccccagtgctgggattaaaggcgtgcactgccacgcccggctagtattagaaaatgttaagaacaaaagtagtctagtcagtcaatgactaaacagacaatgactcctggagtcagtgtgaaaagcatggctaagtggaagtcttccttataggagtcaggagccacctgtttctcacttctcttgtaacttattggaaaatcttagaggagtcagttcccctcttgtcttctgagatggcatattgaaggcaatggacttctattccaacaaggacactgcctctagggtgagtctgtgaacataggcagctggagctctggagtgttgtgagaactgggtagtgtagatgggcaggtggaaggggagtcgcaggaaggctgagtgaggactgtcaggccttggtttggagcaccctgaggttacaggagagtctttactgcctgacttcttgtagttaagttgaagatttaaggtgttagagatagagtgcttttgagtacaaaggactcagacactggtagcatggatgctagagagagtcaaaccttcaagctaccagcagattagaagtggtttttgactgtgttttgttttgttgttgttgtcgtcgtccttgtcgtcgtcatcatcttcttcttctaaagatttatttatttattttatgtgtatgagtacactgtagctgtacagatggttgtgagccatcatgtggttgctgaaaattgaactcaggacctctgcttgctccagccccacttgatcctgccccgcttgctcctgtcctaagatttattatatgtaagcacactgtagctgtcttcagccacaccagaagagggtgtcagatctcattacggatggttgtgaaccaccatgtggttgctgggatttgaactctgctcttccgaaggtcttgagtgctctgagccatctctccagccctgtcttttttttttttttttgaaacagggtctgactgtacaccctggctgggctggaactcactatatatcaatcacaatttatatcaggctagcctctgcctcctgggtgctgaattaaagatgtgtgctaccatacctggcctttgtctccagttttacatcttttaggattctgtctgtctgtctgtccatttattttggctagattgacttttattgtttgttgtgtcttgtgtaacttctctgaatctgcattttcttcatttgaggatgttggtggtgctcttcacagggtttttgtgagttttgaaactgtagaagcacacagtatagctagcactgtgttttgtcttccgagttgtgctcagacatgttagtgagtactcagtgccccagccatgtcccagcttacttcctcaagccttattacctctgcctagtgcaggggtcttgctctttcttggtgatactgcttcttggagctctgctgggcacactccttcattaaggaccacttgagaacccgatccttcttcctctgtagatttctttctctagaaatgttctcaccacagtctgccagggcatcagggttccatgatgccccacctgtatagacttctatcagagtgagtctgtacttgtgcagtgttgaagataaaacccactctcctgtcatgaccagccaagctgacctacaccccaagccctatagctgtatggaccatccatgatagtctcagcacctttctggatgttaagggtgtgtcttcataccaaaggaggactgtcaggccccagtcttggaaaagcctgtgttagaagtcccaggtaataggagtgagtttgtattcttttgctttttagagtttttattccttgcacactgtaggcccagggtgggtgtttatggagtcaagtactcactcctacatcgtatcctcagctgtcagttggggaagtggtggcaagaatctgataagcctgagtgcatcttgtagattttcatctttcactttttaaacctgaattgctggcaccttccggaatccacagcctgagtgtgttcttcacattgccagcaaggttggcaaaagtaatgacaactctggtgtcggcctttaatctcagcactgggaagacagaggcaggcagatcgctgaggccagcctggtctacagagcaaattccaggacatccagggctacacagagaaactctgtcttgctacaacctcccccttctcctaccttggtcccaaaaagtaatgacaactaaagctgtctagttttgcatcctctaggtttgtacatacagttagatttgacttattttgagtttattcattttggaaacttcttgaggaagagcaattcctaccagcttttggtgaagttgtagagtgtttccattttgctttggtttactgttatttaattttatacttagaagattttgttatatttctgtggtggtgtggtctgatagggtgcagatgaatttatttatttatttatttatttatttatttatttatttatttatttttgagacagggtttctctgtgtagccctggctgtcctggaactcactctgtagaccaggctggcctcgaactcagaaatccatctgcctctgcctcctgagtgctgggattaaaggtgtgagccaacactgcccagctgcagatgttgtattgatgtttgtttcatttttaggtctttaaaacatttgatcacatggcccgccaggatgatgagaaaaggaagaaagaactagaagagaaaataagaaaaaaggaggaagaggccaaggccttgccagctgctgaaactgagaaggtagcggtgccggtcccagtgcaggaggtagagatcgatgctgctgcagacttgagtgggcctcaggaagtagagaaggaggagcccccaggctcccaggaccccgagcacacagtgacccatggcctggagaaggcggaagctccaggaacagttagcagtgctgctgaaggccctaaggaccctcctgtgctccccaggtaggagcatctcctgcagtgtcgtcctctctgctgtgcttaagtttgcctatgagtggtttttgttttgtgtggtttgtaaaaaaatatcagctctgttttggtgggcagtggttaactatagaaattcatttcttaatagttctggaggctggaaaccccagattaaagtatgatctgggttgtttgtttgaaggcttctatcagtggtttgcagacagccatcttcctgtgtctcgtcacattcctttgttcttgcctgtgtcttattctcctattctcaagcagcactcaaacaccttagtgagccagattgccttccatcctggtgcttcagggacctcctttcggaactcagtgttgaattc

SEQ ID NO: 2 - DNA, 4002 bp, Mus musculus
atggcagctgcctggcagggatggctgctctgggccctgctcctgaattcggcccagggtgagctctacacacccactcacaaagctggcttctgcaccttttatgaagagtgtgggaagaacccagagctttctggaggcctcacatcactatccaatatctcctgcttgtctaataccccagcccgccatgtcacaggtgaccacctggctcttctccagcgcgtctgtccccgcctatacaatggccccaatgacacctatgcctgttgctctaccaagcagctggtgtcattagacagtagcctgtctatcaccaaggccctccttacacgctgcccggcatgctctgaaaattttgtgagcatacactgtcataatacctgcagccctgaccagagcctcttcatcaatgttactcgcgtggttcagcgggaccctggacagcttcctgctgtggtggcctatgaggccttttatcaacgcagttttgcagagaaggcctatgagtcctgtagccgggtgcgcatccctgcagctgcctcgctggctgtgggcagcatgtgtggagtgtatggctctgccctctgcaatgctcagcgctggctcaacttccaaggagacacagggaatggcctggctccgctggacatcaccttccacctcttggagcctggccaggccctggcagatgggatgaagccactggatgggaagatcacaccctgcaatgagtcccagggtgaagactcggcagcctgttcctgccaggactgtgcagcatcctgccctgtcatccctccgcccccggccctgcgcccttctttctacatgggtcgaatgccaggctggctggctctcatcatcatcttcactgctgtctttgtattgctctctgttgtccttgtgtatctccgagtggcttccaacaggaacaagaacaagacagcaggctcccaggaagcccccaacctccctcgtaagcgcagattctcacctcacactgtccttggccggttcttcgagagctggggaacaagggtggcctcatggccactcactgtcttggcactgtccttcatagttgtgatagccttgtcagtaggcctgacctttatagaactcaccacagaccctgtggaactgtggtcggcccctaaaagccaagcccggaaagaaaaggctttccatgacgagcattttggccccttcttccgaaccaaccagatttttgtgacagctaagaacaggtccagctacaagtacgactccctgctgctagggcccaagaacttcagtgggatcctatccctggacttgctgcaggagctgttggagctacaggagagacttcgacacctgcaagtgtggtcccatgaggcacagcgcaacatctccctccaggacatctgctatgctcccctcaacccgcataacaccagcctcactgactgctgtgtcaacagcctccttcaatacttccagaacaaccacacactcctgctgctcacagccaatcagactctgaatggccagacctccctggtggactggaaggaccatttcctctactgtgccaatgcccctctcacgtacaaagatggcacagccctggccctgagctgcatagctgactacggggcacctgtcttccccttccttgctgttgggggctaccaagggacggactactcggaggcagaagccctgatcataaccttctctatcaataactaccccgctgatgatccccgcatggcccacgccaagctctgggaggaggctttcttgaaggaaatgcaatccttccagagaagcacagctgacaagttccagattgcgttctcagctgagcgttctctggaggacgagatcaatcgcactaccatccaggacctgcctgtctttgccatcagctaccttatcgtcttcctgtacatctccctggccctgggcagctactccagatggagccgagttgcggtggattccaaggctactctgggcctaggtggggtggctgttgtgctgggagcagtcgtcgctgccatgggcttctactcctacctgggtgtcccctcctctctggtcatcattcaagtggtacctttcctggtgctggctgtgggagctgacaacatcttcatctttgttcttgagtaccagaggctgcctaggatgcccggggagcagcgagaggctcacattggccgcaccctgggtagtgtggcccccagcatgctgctgtgcagcctctctgaggccatctgcttctttctaggggccctgacctccatgccagctgtgaggacctttgccttgacctctggcttagcaatcatctttgacttcctgctccagatgacagcctttgtggccctgctctccctggatagcaagaggcaggaggcctctcgccccgacgtcgtgtgctgcttttcaagccgaaatctgcccccaccgaaacaaaaagaaggcctcttactttgcttcttccgcaagatatacactcccttcctgctgcacagattcatccgccctgttgtgctgctgctctttctggtcctgtttggagcaaacctctacttaatgtgcaacatcagcgtggggctggaccaggatctggctctgcccaaggattcctacctgatagactacttcctctttctgaaccggtacttggaagtggggcctccagtgtactttgacaccacctcaggctacaacttttccaccgaggcaggcatgaacgccatttgctctagtgcaggctgtgagagcttctccctaacccagaaaatccagtatgccagtgaattccctaatcagtcttatgtggctattgctgcatcctcctgggtagatgacttcatcgactggctgaccccatcctcctcctgctgccgcatttatacccgtggcccccataaagatgagttctgtccctcaacggatacttccttcaactgtctcaaaaactgcatgaaccgcactctgggtcccgtgagacccacaacagaacagtttcataagtacctgccctggttcctgaatgatacgcccaacatcagatgtcctaaagggggcctagcagcgtatagaacctctgtgaatttgagctcagatggccagattatagcctcccagttcatggcctaccacaagcccttacggaactcacaggactttacagaagctctccgggcatcccggttgctagcagccaacatcacagctgaactacggaaggtgcctgggacagatcccaactttgaggtcttcccttacacgatctccaatgtgttctaccagcaatacctgacggttctccctgagggaatcttcactcttgctctctgcttcgtgcccacctttgtggtctgctacctcctactgggcctggacatacgctcaggcatcctcaacctgctctccatcattatgatcctcgtggacaccatcggcctcatggctgtgtggggtatcagctacaatgctgtgtccctcatcaaccttgtcacggcagtgggcatgtctgtggagttcgtgtcccacattacccggtcctttgctgtaagcaccaagcctacccggctggagagagccaaagatgctactatcttcatgggcagtgcggtgtttgctggagtggccatgaccaacttcccgggcatcctcatcctgggctttgctcaggcccagcttatccagattttcttcttccgcctcaacctcctgatcaccttgctgggtctgctacacggcctggtcttcctgcccgttgtcctcagctatctggggccagatgttaaccaagctctggtactggaggagaaactagccactgaggcagccatggtctcagagccttcttgcccacagtaccccttcccggctgatgcaaacaccagtgactatgttaactacggctttaatccagaatttatccctgaaattaatgctgctagcagctctctgcccaaaagtgaccaaaagttctaa

SEQ ID NO: 3 - protein, 1333 aa, Mus musculus
MAAAWQGWLLWALLLNSAQGELYTPTHKAGFCTFYEECGKNPELSGGLTSLSNISCLSNTPARHVTGDHLALLQRVCPRLYNGPNDTYACCSTKQLVSLDSSLSITKALLTRCPACSENFVSIHCHNTCSPDQSLFINVTRVVQRDPGQLPAVVAYEAFYQRSFAEKAYESCSRVRIPAAASLAVGSMCGVYGSALCNAQRWLNFQGDTGNGLAPLDITFHLLEPGQALADGMKPLDGKITPCNESQGEDSAACSCQDCAASCPVIPPPPALRPSFYMGRMPGWLALIIIFTAVFVLLSVVLVYLRVASNRNKNKTAGSQEAPNLPRKRRFSPHTVLGRFFESWGTRVASWPLTVLALSFIVVIALSVGLTFIELTTDPVELWSAPKSQARKEKAFHDEHFGPFFRTNQIFVTAKNRSSYKYDSLLLGPKNFSGILSLDLLQELLELQERLRHLQVWSHEAQRNISLQDICYAPLNPHNTSLTDCCVNSLLQYFQNNHTLLLLTANQTLNGQTSLVDWKDHFLYCANAPLTYKDGTALALSCIADYGAPVFPFLAVGGYQGTDYSEAEALIITFSINNYPADDPRMAHAKLWEEAFLKEMQSFQRSTADKFQIAFSAERSLEDEINRTTIQDLPVFAISYLIVFLYISLALGSYSRWSRVAVDSKATLGLGGVAVVLGAVVAAMGFYSYLGVPSSLVIIQVVPFLVLAVGADNIFIFVLEYQRLPRMPGEQREAHIGRTLGSVAPSMLLCSLSEAICFFLGALTSMPAVRTFALTSGLAIIFDFLLQMTAFVALLSLDSKRQEASRPDVVCCFSSRNLPPPKQKEGLLLCFFRKIYTPFLLHRFIRPVVLLLFLVLFGANLYLMCNISVGLDQDLALPKDSYLIDYFLFLNRYLEVGPPVYFDTTSGYNFSTEAGMNAICSSAGCESFSLTQKIQYASEFPNQSYVAIAASSWVDDFIDWLTPSSSCCRIYTRGPHKDEFCPSTDTSFNCLKNCMNRTLGPVRPTTEQFHKYLPWFLNDTPNIRCPKGGLAAYRTSVNLSSDGQIIASQFMAYHKPLRNSQDFTEALRASRLLAANITAELRKVPGTDPNFEVFPYTISNVFYQQYLTVLPEGIFTLALCFVPTFVVCYLLLGLDIRSGILNLLSIIMILVDTIGLMAVWGISYNAVSLINLVTAVGMSVEFVSHITRSFAVSTKPTRLERAKDATIFMGSAVFAGVAMTNFPGILILGFAQAQLIQIFFFRLNLLITLLGLLHGLVFLPVVLSYLGPDVNQALVLEEKLATEAAMVSEPSCPQYPFPADANTSDYVNYGFNPEFIPEINAASSSLPKSDQKF

SEQ ID NO: 4 - DNA, 30 bp, artificial
  primer
gcgggatccgaaccggtccagctacaggta

SEQ ID NO: 5 - DNA, 32 bp, artificial
  primer
gcggaattcctcgaggatgggcaggtcttcag

SEQ ID NO: 6 - DNA, 26 bp, artificial
  primer
gcttcttccgcaagatatacactccc

SEQ ID NO: 7 - DNA, 27 bp, artificial
  primer
gaggatgcagcaatagccacataagac

SEQ ID NO: 8 - DNA, 24 bp, artificial
  primer
tatcttccctggttcctgaacgac

SEQ ID NO: 9 - DNA, 22 bp, artificial
  primer
ccgcagagcttctgtgtaatcc

SEQ ID NO: 10 - DNA, 25 bp, artificial
  primer
cctccctattccccaagatgtatgc

SEQ ID NO: 11 - DNA, 22 bp, artificial
  primer
ggagaggctattcggctatgac

SEQ ID NO: 12 - DNA, 25 bp, artificial
  primer
ctgggctccctcttagaataaccta

SEQ ID NO: 13 - DNA, 22 bp, artificial
  primer
ggagaggctattcggctatgac

SEQ ID NO: 14 - DNA, 21 bp, artificial
  primer
ctctgagcccagaaagcgaag

SEQ ID NO: 15 - DNA, 23 bp, artificial
  primer
gaccagagcctcttcatcaatgt

SEQ ID NO: 16 - DNA, 22 bp, artificial
  primer
gagaatctgcgcttacgaggga

SEQ ID NO: 17 - DNA, 32 bp, artificial
  primer
gcgaattctatgtctgggggcaaatacgtaga

SEQ ID NO: 18 - DNA, 37 bp, artificial
  primer
gcggatccttatatttctttctgcaagttgatgcgga

SEQ ID NO: 19 - DNA, 57 bp, artificial
  synthetic sequence
ttggggtcattgtcgggcattggggtcattgtcgggcattggggtcattgtcgggca

SEQ ID NO: 20 - DNA, 88029 bp, Homo sapiens
gatcatgaggttaggagttcgagaccagcctggctgatatggtgaaacgccgtctctactaaaaatacaaaaattagctgggcgttgtggcaggtgcctgtaatcctagctacttgggaggctgaggcaggagaattgtttgaacccaggaggcggaggttacagtgagccgagatcacgccattgtactccagcctgggcgacaagagtgaaactcccatctcaaaaaaaaaaaaaaaaaaaaaaaaaaagacatgtattctctctctcagtcacggacggcagaagtccgaagtgaggagtgggcagggctgcacttcctaggctctcggggagactttttttcctgtcccttccagtttctggtggctccaggcatgccttggcttatggcagcattattattccagtgtctgcctctgtgatcatagtgcctccttttcttttttttttttttacattttttttttgtatttagagaaaaaaacacttaacataaaatttaccatcttaaccttttttttgagactctgttgcccaggctggaatgcagtgttacaatcacagctcactgcagcctcaacctcctgggctcgtgacatcctcccatctgactctcccaagtaactggggaccactggcatgtgccaccacacttggctaatttttacattttttgtagagacagggtttctctatgttgcctaggctggtctcaaactcctcagctcaagcaatcctcctgccttggcctcccaaagtgctgggattataggcgtgagccaccacgcctggccatgttaaccatttttaggtgtgcagttcagtatgttaaatatattcacattgttatgaaacagatgtccagaactttttcattttgctaatctgaaactctgtacccattagacaacagctccccccgcaggtaaccattctactttttgcttctatgattttgactactttagacactttatctaaatggaatcatatggcaattgtctttctgtgattgacttatactacttagcataatgttaagtttcatccatgttgtagcatgaatcagaatttcatccctttttatggcttgataatactgcattgtatgtatataccacattttgcggtaggtacaatgtatatttacattgcttccacctcttggctactgtgaataatgctgctatgaaaatgggcgtgtaggtatcttttccagatcctgactttacttcctttggataaatacttacaggtgggactgctggggtatatgattgttctacttttaattatttaacactcttctacaatttattttctgtttttgttgttctaatagtagttattattaggtgaggtatttcttatctcttataaggacacctgtcattggatttagggtccacctggttaatccaggattatcaagtctcaaaatcctgaattacatctgcaaagactctttttccacataaggtcacattcacaggttccagtgattcaaacatggacatgtcttctggtcccccattatgtccactatactctcttttttttttttttttttaagatggagtctcgctgtgtcgcccaggctggagcgcagtggcgcgatcttggcttcctgcaagcccacctcccaggttcacgccattctcctgcctcagcctcccgagtagctgggactacagacacccgccaccacgcccagctaatttttttgtatttttttagtagagacggggtttcaccatgttagctaggatggtcttgatctcctgacctcgtgatccccctgcctcagcctcccaaagtgtcgggattacaggcgtgagccactgcgcctggcctaagtccactatactttcttcttccctgccttatttttattcttgatacttatctccatctgacatgctctatatttctttatttatcttgtttggcagacgacaatcaagataaagccatggagacaaggatttttgttgctgttgttcttgttttttgagacagagcctcactctcacccaggcctagagtgcagtggcacaatctcggttcactgcaacctctgcctcccaggctgaagtgatcctcccacctcagcctccagagaagctgggactacaggtgcttgccacatgcctggctaattttttgtatttttggtagagacggggttttgccatgttgtccaggctagtcttgaacttctgagttcagatgatccacccaaagtgctgggattacatgcgtgagccactgcgcctggcctagacaaggatttttgttttggtcacctgtgttttcccattagaacagtggctggcacaaatggctgcacagcacatactggttgaacaaatgaaggacggggtggctggtctagacaaagagcctagacaaacatcggcagaaattgcttcatggcttctgagcagaaaaatctctcatctggggaattagactccctaagttaaattttctttcttttttggagatggggtctcactctgttgcccaggctggagtgcagcagcaccatcacagctcactgcagcctcaacctcctgggggtcaagcaatcctcccacctcagcctcccgagtagttgggactacaggcccatgccaccatgcccagctaattttttttttggtagagacagggtgtcaccatgtagcccagactggtcttgaacttctggactcaagcgatcttcctgcctcggcctcccaaatgctgggattacaggcatgagccacagtgcctcacctcctaagttaaattttctgcagtggagaatacaatctctttaatattatctctcagttaagacaaatttcaggatcctccttaaaaaaaaaaaaaaaaagaaagaaaataagtttgccaatacaaataccatttctcactaaagtgaattagggttccttggagaaatggttggttttgtttctgggcagtaaatgtataaaacggaaagcaaggaagtccagggtgtccaatcttttggctttcctgggccacactggaagaagaagaattgtcttgggccacacataaaatacactaacaatagctgatgagctaaaaaaaaaaaaatctcataatgtttttagaaagtttatgaatttgtgttgggctgcatgggccacaggttggacaagcttgacctaaagactactaggattgtggattactaggattgtgccagaaggacacagcagcaactaaatatttgatgagacaatctgaacatttaaaaaaggacaatgactgtaatggattaaagcacatcaaatatctaaacatccatcaattcatgatagcactgccccttctccccaaagaacccaaagtggtcacagttagaggttgctggggcatccatccatccatctttattattattactattatttgagacaaggtctcactcagtcacccatgctagagtgctgtcgtcccatcacggctcactgtatcctcaacctcctgggctccagccatttccctgcctcagcctcctaagtagcgggaattacaggcatgcatcaccatgcctgtctaatttttacatattttgtaaagatcttgccatttcctgggctcaagcagtcctcctgtcttggccccccaaagtgctgggattataggcagagccactgtgcgtgggaaagcatcaagcatacatcctggctatcccgaatggattgtatttcaaagtaaccagagagatgagggaatgctcacctttgtagaagaatctcatcttataaatgcaggagaaatgagagcatttgaaattaccactttgcacacctaaggaacatcataaaactacactagggtttctcaacctgggtactactgacattttgagctggataattcttcgctgtgggggggaggtgtgctctgtgaattatacaatgtttagcagtattccttgcattcattttctagataacagcagtaccacccgccaccccccacccagttgcaacaatccaaaatatctccagacattgccaaatttccccttggtaggacagggcagaatcaaccctcgttgagaaccaatggtctaatgatcatcaacgtttgctagactattagaagaaaggctgatggtaaacttcatggataatcaggatgacaacccccaaattgagagatgaattacaatattactaagagacaaacccgccttgtgcctcagtagaagtacatagtgccagccacgaagcgttattgcaaacaaaacaaaacaaaaaaacccaaacctcaacattacacctaaacctaatgaagcttctagccaggggcaaatccaagctttgtggggccttaaactatacaaatttcacagtcctctttaagaaaaagacacaaaattataaatgcgaaattaggtacgggggtctatgcaagggagggcctgaagattaagcttcattagtttcactgtaaacctcccctgactctagaattaactgtgattacaggacataccagggacaaaaaacgttaaatgacacctgaagatacaatcagcaaaacccagaaagtggaaaattctgttggtcaaatgacccagtttcttcaataagtaaatgccatgaataacaaacaacaaaaagagaggggaaatttatatatataatatatatataatatgtataatatatatatatgttgttatatggtttgtttttttttttttggacacgagtctctctctcacccaggctggagtgcagtggcatgatctcggctcactgcaacctctgcctcctgggttcaagcgattcttctgcctcagtctcccaagtagctgggactacaggtgagcaccaccacacccagctaatttttgtatttttggtagaggtagggtttcaccatattggccaggctggtctcgaactcctgacctcgtgatctgcccaccttggcctcccaaagtgctgggattacaggtgtgagccactgcgcccggccctgtttttgtttgttttgagatagaatctcactctgttgcctaggctggagtacagtggcatcatctcagcccactgcaacctccacctccctgattctagcaattctcctgcctcagcctcccaagtagctgagattacaggtgtgcaccaccacacctggctaatttttgtatttttagtagaaacagggtttcaccatgttggccaggctggtctcaaactcctgacctcaagtgatccttccacctcagcctcccaaagtgctgggattacaggcatgagccactgtgcccagccaaaattgttatatattaagagacatatatttgtatgaaatgcagtaagtaaaccttgtttggaccctaaatatctaatgtacaaaattttttaaggcaatggggaaaattaaacacatactaggtattaagtgatgttaaataatttttaaaattttggtgggtgtgataatagtataaagtccttatctgttagagacacacactgacgtatttataggtgaaatgacatgatgtccaggatttgctttaatatacagcacttcaaaaaaaaatgcagaaagggatacatgaaatgagaaaggcagcaaactgttgttgaagttggatgatgagtacagccctccgttattatccaagggggataattcctggacccctacggataccaaaatccaggtatgctcaagttctttatgaaagttcattgtaattattctattatataaaagtttcgaaattctgttgataaaatgtatttttagtagagacgggggtttcaccttgttggcccaactggtctcgaatttctaacctcagatgatccaccagccttggccgcccaaagtgttagcattacaggcgtgagccaccgcgcccgcctggccttgataaaaacagttttaaccttccgttgcttcgattccatgcccactaagtaacattccagtttgtttttcactttcaaaaggatgtgctgtaactaggggatgtaaacaagctccatgaccctacttattccaagttttcgttccactctcccaccttttttttaagacaaggtatcaccctcggtcgcccaggctggagcgcgatcactgcttaatgcatcctcgacctcctgggttcaagcgattctcctgcctcagcctcccaagtagctgggactacatgcgcacaccaccacaccggttaattttttgtagagacgggggtttcaccatgttgcccaggctggtcccgaactcctgggctcaagggatccgcccgcctcagcctcccagagtgctgggattacaggtgccagccaccgcgcccggccccagcttcttaaaagaatgatccgaaactatggcagcactgggcttttggtccccacccaagaaatgcccgctcgcagaggctcgccgcggcaggctctcccgacgtgacagagtgtgggtctggattcagcctcggttcttacgagtcagataggtggacacgcaaagcaaaacatcacagggctttttgtatttagcacagaaaacacttgtgagcccgagctgagaacccaaaaggcacgcttcaggccatcgtagccaccaagcctggtcagattccgtccaccgtctccttggtgctccgagacccaaatcgctgactggggccgagggcgggcgtgactgcgcaggcgtgcctcccctgcgagatgccggaggtaagctgcggggtaaggggcgagaaattaagggcgaacgtcattgcgcatgcgccctctactctcgttgcgggggtaggcgggcgccgggctgtgtgagggggcggggcgcggcagtgttcggtacggatggagttgcaggagacggcgagtacatatcactgcgcaggcgtcctcttcccctaactctcagggtcgctagggtggcgcgcaggcgcagagcgatgcgcaaatgtgcgcaggcgcttaggggctgaggcgcgatggcaggtgtcggggctgggcctctgcgggcgatggggcggcaggccctgctgcttctcgcgctgtgcgccacaggcgcccaggggctctacttccacatcggcgagaccgagaagcgctgtttcatcgaggaaatccccgacgagaccatggtcatcggtcaggcgggctgagggtggggaggccctttgtacccagctcagccctcggcggcgctccctcctcccgagcccagccgggtcgctggctcccccagtacctagcctgagggtgccccgaggacgccaggccccctgcctagagctccgggccgcacgtcggagggggccgggcggagaggcggcccactagggccggtcgtgactatgtgtctgccccgcaggcaactatcgtacccagatgtgggataagcagaaggaggtcttcctgccctcgacccctggcctgggcatgcacgtggaagtgaaggaccccgacggcaaggtaaggctggcgttggcccacgcagccgttcttcagtggagctcccgtggggtgtaaagcactgcctggaggaggcctcaagggacaggaacttgcacttggagagcctgcggtataaaggtggggccttcactcacatatgttgcaggtggtgctgtcccggcagtacggctcggagggccgcttcacgttcacctcccacacgcccggtgaccatcaaatctgtctgcactccaattctaccaggatggctctcttcgctggtggcaaactggtaagaggattttctctttggcttcagcttagaatctctcacttgtttccaaattttgatttatcaagattgtgaaactttgtagcacagtcagaattggggagacagatgttgccttctgctccacagccagggacaatagtgggttccataccctggaacagacaactggaggccccaccactcatacattccatgtttccttgtagcgggtgcatctcgacatccaggttggggagcatgccaacaactaccctgagattgctgcaaaagataagctgacggagctacagctccgcgcccgccagttgcttgatcaggtggaacagattcagaaggagcaggattaccaaagggcaagtgcatatctccttgtaatttgagagggcagttgacctttatacccactatacctactcaagtttctgcttgggagatcagctctgcagagaatggaatgagaagtattggtttagataggttgtttgtttgttgtttttgagacggagtttcactcttgttgcccatgctggagtgcaatgccatgatcttggctcactgcaacctccgcctccccaggttcaagcgattctcctgcctcagcctcctgagtagctgggattacaggcatgcgccaccatgcctggctaattttgtacttttagtagagacgggggtttctccacgttggtcaggctggtctcgaactcccgacatcaagtgatccgcccgcctcagcctcccaaagtgctgggattacaggtgtgagctaccgcgccctgcctgttttgcttttttatcaaaacattttattgtggtaaaatataacaccaaatgtgtcattttaactgtctatatagttcagtggtattaagtgccttcataatgttgtgctaccaacaccatcatccagctccagaactttttcatcttctcaaactaaaaatctgtacttattttgttttgtttttgagatggagtctcgctctgttgcccaggctggagcgcagtggcgccatctcggctcactgcaccctccgcctcccaggttcaagcgattctcctgcctcagcctcccaagtagctgggattacaggcaagtgccaccatgcgtggctcatttttgtgtttttagtagagactgggtttcaccatgttggccaggctggtcttgaactcctggcctcaggcaatccactgccgcagcctcccaaagtgttgggattacaggcgtgagccactgcacccagcaaatctgtacttattataaacaataacttcccgtttccttttgtcctgacacccaccattctactttctgtctctatgatcctgactaccctatctcatataagtggaatcattcagtatttgtccttttgtgactggcttatttcactgagtataatgttctcacagttcatccatgttatagcatgtgtcagaatttcttaaggctaatattccattgtatgcatgtgccacatttcgctttcagtagtcatttttaagctctataaaataaaatgaagaaaggacagttcacaatctagtaatagccattgcctacctgtttttcttggactcttgttggaaatggtaggatcatgatttcagtcctaacagagatgcttgtggagggacagcctgtccctttcttggggcagcctcagtggggagaccatagcactcctaatggagtcacagatagtattccaaaaggagtttggtcctggagttgagtaattacacgcagggagggacctcacaacagccagactgtttctcctgctcacttaaccctgtgttgccccacacagtatcgtgaagagcgcttccgactgacgagcgagagcaccaaccagagggtcctatggtggtccattgctcagactgtcatcctcatcctcactggcatctggcagatgcgtcacctcaagagcttctttgaggccaagaagctggtgtagtgccctctttgtatgacccttcctttttacctcatttatttggtactttccccacacagtcctttatccacctggatttttagggaaaaaaatgaaaaagaataagtcacattggttccatggccacaaaccattcagatcagccacttgctgaccctggttcttaaggacacatgacattagtccaatctttcaaaatcttgtcttagggcttgtgaggaatcagaactaacccaggactcagtcctgcttcttttgcctcgagtgattttcctctgtttttcactaaataagcaaatgaaaactctctccattaccttctgctttctctttgtccacttacgcagtaggtgactggcatgtgccacagagcaggccctgcctcactgtctgctggtcagttctgggttcacttaatggctttgtgaatgtaaataaggggcaggtcttggccctagaggattgagatgtttttctaaatcttagaactatttttggataaattatatattttccttcctagtagaagtgttactgcctgtaactagctcaaaataccaatgcagtttctgcattctgggttttgtttttccttttttttttttttttttttttttgagttttgctcttgtcgcccaggctggagtgcaatggcgtgatctcagctcactggcaacatctgcctcccgggttcaaatgattctcctgcctcagtctcctgagtagctgggattacaggtgcccgccaccacgctcagctaatttttgtatttttagtagagatggggttttaccatgttggccaggctggtcttagactcctgacctcagttgatccacctgcctcagcctctgcattcagtttattcacatatttttggtaactcccatggcagctcctaggatttcagcggtctgtgggccagaaagcaggcaccagggctgacctcaaggccgtatcagagggccaagcagagttcttttggatacctgcttttcatcccacagggccttagagtcagaggtaaggtagcaacagagctagaatggggcaatgcactcttaccctccttctcaacttttatttaagctgtgctaaatgttttcttcaagggaaccagatttagttctttacagaattttccagtgaaataaaacatgttgtaatagctgtgtttgagatgaaataagaggttgtgggtagaggggaggcacctaaaggaaaagaggaaaggtgcctgggctacctatgcagataacctggagtggacttcactgtggactcgtggtactaaggcttggcctggacaggcagtctagggggtatgggaatacacggtgtggttgttcaactatttgcaaaggtcaaccaaatagaccacatgttcgcaaagtatcatctgaggaaattaagtaccttcttagccctctcagtcataaatttgaacaaattttaatacacttccctcatgcccttctatataaaacttaataccattagttccccattcttgacattttatttcagtttttattatatatttatttgaaatatttattaaattatctgacctacagaactaaattcttctccttttgttatttcttatgtcctataccatatatgtacctatttatatatatatttatgtatttttaaaatttttatttattttattttttgagacagtcttgctctgtcgcccaggctggagtgcagtggcatgatcttggctcactgcaacctctgcctcccgggttcaagcagttctgcctcaacttctgagtagctgggattacagacacccaccactacacccggctaatttttgtatttttattttatcttattcatttatttatttttgagatggagtctcactctgtcgcccaggctggagtgcagtggtgcaatcttggctcactgcaacctccacctcctgagttcaagagattctcctgcctcagactcccgagtagctgggattataggcgcccgccaccatgcccagctaatttttgtatttttagtagagacagggtttcaccatgttgaccaggctggtcttgaattcctgaccgcaggtgacccgtctcgcctcccaaagtgctgggattacaggtgtgagctggccgggcacaggtgatggggtcttgctctgtccccaaggctggagtgcagtggtgccatcacagctcaaagcaaccttgagctcccaggttcaagtgatcctcctaccttaccctcccaagtagctggtactacaggtatactccactgtgcctggctatttttactcttaaaaatacatgtgggctgggcacggtggctcacgcctgtaatcctagcactttgggaagccaaggtgggtggatccctatagcccaggagttcgagaccagcctgggcaacatggcgaaatcttgtctctgcaaaaaatacaaaaaatttagctggtggcacatgcctatagtcccagctacttgagaggctgaggtggaaagatcacttgagcccgggaggtcaaggctgcggtgagccatgatcgtgccactgcactccagtctgggcaacagtgatcccatctgaaaaaaaaaaacaaaaaaaaaaatgcaatttagggccaggtggggtggctcacgcctataatcccagcactttgggaggccaaggcagggggatcgcctgaggtcagcagtttgagaccaggctggccaacatggtgaaaacccctctctactaaaagtataaaaattagccaggcatggtagtgtgtgcctgtaatcccagctattcaggaagctaaggcaggagaatcgcttgaacccgggaggaggttgcagtgagcagaaatcgagccactgcactccagcctggggggcagagggagactctgtctcagaaaaaaaaaaaaaaatgcaatttagttctctaggcttttccatttaatagttttatatcctcctgtttctaaatctggatgacagtgtaacactccagtaaggtgaattgtgaattgctgaaattcttcagatgtttaaaagagttttcagtattcctcatgttagaattaatgcagagaaaaattttatcctttgaactagttacatgttgtggacttctggcctgaggctcttggggattatgtgacatattgggaagggacacatttctgctctgtggctgttactagaaatctagccagcaaatcagactacgtttgtgagaagacaggaaggcacagattagggttgagccagccttcaacaggtttggctggcagtagacacagtggagcacatcttaactattttggtaggtcctgggtttctcttggtagtttttgatagaaaggggaatggtgtgaggaaaaagtgggcatacatttcacctttccactgataaggcaggtggaattgggatagtcagtggatgggccaatagctggtggctgtgagaagaataaggatttccatactggtgtgtcatatttacagataggttgtgacctaaaaagttttttaaaaaacagcagttagggcctgggcgcggtggctcacgcctgtaatcccagcactttgggaggccgaggcgggcggatcacaaggtcaggagatcgagaccatcctggctaacaacggtgaaaccctgtctctactaaaaatacaaaaaattagccgggcgtggtggcaggtgcctgtagtcccagctactcgggaggctgaggcaagagaatggcgtgaactcgggaggtggagcttgcagtgagctgagatcatgccactgcactccagcctgggcgacagagtgagactccttctaaaaaacaaaaacaaaaccaaaacagtagttagggtacacacacacaaattctagtgattttccccccaatactacccttgacttttgaaattcttgctttctcagagtttacaacatccttaccaaacagccttctccctccttaccacaaaaaaagaaaaaaaagttctggggttgaggggacactccattcttaacatcctctattatcccagcccaattccccagctctcactgggactagttgtacctatcttcatcatttggtcccagcatgactacctgttggtgcatgagctgatctctcctaacctaacagccagatgctagtctctggtactcagatgctgggctgcatcagataggatgcacaggatcatcctggaagcttgttgacatagattcctgtgcaacactcagatatagtcttaatgtagatttgtgttgggtggtatggtaggtagaataatggcctaccactctgaaacatatgaatatgttacctaacatgacagaagagaattaagttgctaatcagatgactgtaaaataaattatcctggatcatctggatgggcctaatgtaatcacaaaggttgtttccttgccttttccagcttggctctggctcccttcctccagcaagggtgggttgagctctcacatggcaccactttgacctcttctgcttccctcttctacactgaaagacttatgggccaggagcagtggctcgcacctgcaatcccagcactttgggaggccgaggaaggcagatcgcttggccccaggagttcaaaaccgtcctgggcaacgtggcgaaaccccatctagaaagaaaagaagagaaggggaggggggaggggaggaggagttacatatatacacatacacacacacacacacgtacgtacatacatacatacacgctaactggacgtggtggtgcgtgcctgtagtcttagctttccaggagactgaggtgggaggaccacttgagcctgagatcgcgccagcctgggtgacagtcagaccatgtctcaaaaaaaaaaaaagatttgtgattaggattcttagtcctcacctgtattattttcctattgctactgtaacaaattaccacaaatttactggcttaaaacgacgcaagtctgtaggtcagaagtctgacacgggtcttaactggtgacccgagtcagatttgggacacaaagaacagaaaccaagctgtgcaggtttctgacaggcagtccggttagggagccctacagcaacccgccggtcctctctctcaggcagttgctgccatggctcattattccaaccggttctcctcagcccagtctatctcagtggctccattcatagggtgatgtgcccggcgggacactaaccctaaccaagcagagagacggtcatgcccgtcacgacctcggccctcgccccggccgaggcttctcctgcaggtcgcgagaatcaggtgcgtcagcggcgtccgggaacgccggaagagccagtggagcggctctgtagtccaaagtaccccgtcgaccccagcacggccgctccaccgcctcctactagacccagtcctagggactgcgcagtcgcagagctccgtccgagtaccggaagcctaggccgccagcacttccgggaagtgacttcgtctccgaagccgattggttgttgctttgctcccgctcgcgtcggtggcgtttttcctgcagcgcgtgcgtgctgcgctactgagcagcgccatggaggactctgaagcactgggcttcgaacacatgggcctcgatccccggctccttcaggtacacgcgagggctggggagccggcttacgggctctgcggggcgcgccatcgctcttcacgccgcttaaaccgcactcctggtctcctaggctgtcaccgatctgggctggtcgcgacctacgctgatccaggagaaggccatcccactggccctagaagggaaggacctcctggctcgggcccgcacgggctccgggaagacggccgcttatgctattccgatgctgcagctgttgctccataggaaggcggtgggtaacgagagagctgaggggaggaaggaggcaagctccaaaagcctgggaagggcggttcccgtttgtctgaggttttctcttggccctgtacccgtgcaggccggcctgagaacctggtgctgttgtggcaaacactctgggctggagttcaggttacctggatccttgtccggccctgctaccaccaacctttgcgtaatcttcgacaaagcactttcttttctttcttacataaaaagggagcacatctatcttttctacttacagaattattgtgagaatttagcttcataactagtatatttaaagtagcttcataaacatcagagtacgttattctttttgagggtcagtgcctggggaaagaactctccactctgcattctgaggcgggcagagtgatagatgatcaaagtactgctaagtagtgttgcagcagatgggtcaggtaggctggaaggggtagagacacgtggacacagtgatgtgcactgctggctaaagtctttaattcatattcttacagacaggtccggtggtagaacaggcagtgagaggccttgttcttgttcctaccaaggagctggcacggcaagcacagtccatgattcagcagctggctacctactgtgctcgggatgtccgagtggccaatgtctcagctgctgaagactcagtctctcagaggtgggtaaaagcagcaaagctgtacctgaatgaagctacacagtgttgtggggttgggtttgtgtgtggcaaaaaagagagcaaatccagggtgagatcccagctgctacattctgcctgatactgatgtcttgtccacctccagagctgtgctgatggagaagccagatgtggtagtagggaccccatctcgcatattaagccacttgcagcaagacagcctgaaacttcgtgactccctggagcttttggtggtggacgaagctgaccttcttttttcctttggctttgaagaagagctcaagagtctcctctggtaaggcagaggtgggtgtgattcctagtggaaacatctgtgagtaggagttgggacgagagcggggtggctggaagccagttactacaattagcggcccttggagctggaatctgattggattctttcatttcagtcacttgccccggatttaccaggcttttctcatgtcagctacttttaacgaggacgtacaagcactcaaggagctgatattacataacccggtaagaggcaccatggaagtgtctggagctgcagacatgggggcactcaaagatcttgatgctccttcttaggggattctttggtgttttgggtgggacagttgtcacttagtgtctcatccctggtcctgaggcactaaaagccagtggtctaaaatcactatatatttccaagtgtccacaagggatgtctcccatttcaggccatgctttgcctaaaatcctgagcaaggacctcccctaaggggcagctttgagcagcagagccaaaattctaaggccaaggttctcatcttaagtaaactttacctttcagaaggcctgttgctgtaggccttcccttctcaatgtagtcctttattgatgtgtttctctttgttctgtgcttggaagtattttatatatggtttatatggtatactctatataccacaacaataagggcattttggggttttaggttacaaaactggaggagagttagggtgccaggaatccttaaatgcatctctgccctgcactaaaatgttgatgctttggttggtgagtaagtggccatacatctctgtgttcttttcctttctgaccacaggcctgttttctcccccaggttacccttaagttacaggagtcccagctgcctgggccagaccagttacagcagtttcaggtggtctgtgagactgaggaagacaaattcctcctgctgtatgccctgctcaagctgtcattgattcggggcaagtctctgctctttgtcaacactctagaacggagttaccggctacgcctgttcttggaacagttcagcatccccacctgtgtgctcaatggagagcttccactgcgctccaggtctgccacagccaacatcttggttgaaataagttgaagatagagatggaaaggggacccagttaatgttctgtttcttaagcacttagtaggggccaggttctagatgtgactgatactgacttctcccaactccaaaatacctatcatggccgggcaccatggcttatgcctgctgtaatctcagcactttgggaggccgaggtgggcggatcgcctgaggtcgggagttcaagaccagcctggccagcatggtgaaaccccgtctctactaaaaatacaaaaattagctggacatggtggcaggcacctgtaatcccagctactcaggaagctgagataggagaattgcttgagcccgggaggtggaggttgcagtgagccaagatcgtgccattgcactccagcctgggcaacaggagtgaaactctgtctcaaaaaaacaaaaccctataattatttccagctgaggaaactgaggcacaatgattaagtagggaaagagattaagaagaggaaaaaggaaagggtgatggttactgtgatactagggatggcagaggggccttgagcttgctctgctgagctgattctctgtccgctcttggctgcaggtgccacatcatctcacagttcaaccaaggcttctacgactgtgtcatagcaactgatgctgaagtcctgggggccccagtcaagggcaagcgtcggggccgagggcccaaaggggacaagtgagtccatgcctctttttccatccctccccagaaatgcctgtgtttttagctttttggaagactaaaaccagagtgcacagagcagggagccaaaccttccaggcctggctggtagtgtagcccagagagccccacaggttcttgctcagctgcctggatatagagaagggagtggatggtgcacactgcacatgcaccacgaagggcaaaactgccggggttgttggcatgcagagccctgcaggggagatggcccatcctgcattggtggtatggctgtgacttgcagggagcatatttctgaagggaaaaggaaccccccaactctccagtctctgtccagctgaaggcttgactagctcagagttggttttcagatcaccatgtagggcaatgagttctgctgttgtcccagaacagaggtcaggccgagatttgggtacatgtcaaagctccaggctgccccaggaaaccctgactcctggaacggttccattgttggagagtcctctgtatgtcagggtcttatgatctacaggcatttagaggaagttttgctgattcagcgtgtgaatacgtgcccagaggagaggaagggtccggctgacattgagttatctctgcagggcctctgatccggaagcaggtgtggcccggggcatagacttccaccatgtgtctgctgtgctcaactttgatcttcccccaacccctgaggcctacatccatcgagctggcaggtagtagtgtgacggcccaggcatctgcatggtaggcacactgagggacttggggtgtgctggacagagcctgcgggttggagatgcaagctgcactgtcttcccttgcaggacagcacgcgctaacaacccaggcatagtcttaacctttgtgcttcccacggagcagttccacttaggcaagattgaggagcttctcagtggaggtaagagcctggctcttgtggtcctgggccagggtcaggcttcttccacaatgctttaaaactccatgataatgatgacagaggtcacaacatagtgtgacaggccacttccaccatccatccttgttctgccctgagtggcaggcactgtcccccttgagagataaacaaattgaggtaatttgtccaaagttgtgtttactgtctgcctcatgagcgttgagtgacctgacaggctgctgtgacagctcaggacagcacctgaccccagggtgctgggtggtcctggactgctctctgtggccgtcgtcatgggggtaccttgactcccaaggaataccatggggtactccttgggagaggagaagagagtgggtgacgggttcttgggcttggggccacacaggccacccccatccacacacggggacagatgggtcatcactgtaagaggcccaggtgcagctaacctgcatgttcggcatcccaggaaggcggtgggtcccctgctgctttcccccaagggggaggtgcaggaggcctccaatgaagaccctatcctaaggcctcagcctgtgggaccctcgctgctttcttctccacagagaacaggggccccattctgctcccctaccagttccggatggaggagatcgagggcttccgctatcgctgcagggtgagctgctgtggtggggaggggaatgagaggggaggggctgtggcccagggattgcaccgtcttgctgagcatccaggtgtgaagggaggatttggggcagcctcactgtcttgaccttcagtgtccacccccaggatgccatgcgctcagtgactaagcaggccattcgggaggcaagattgaaggagatcaaggaagagcttctgcattctgagaagcttaaggtgagtggatgggaggtgagaaggggatagatcttagacggctgccctttttggagactggctgagctccgagtggtgagaagcagagaactgggcagttttctggcctttggcacggaaggggaggaaatggacccagaatcatggaaggaagccagtctgttctgcttggtggtaaattggcacaaccttatggtggacactgtccagcagaattacgagctcatgtgtcctttcatccgaaattccacttctggaacttaatcctggtcacgcttgtgaatgtgcacagtcaagcatgtgcctgcattcatccatccatggcattatcatggaaccaaaagatggaaacagcctggggccaccatagggggcttgctaggtaaactcaggtgcattcagagccgaaggttacatgggaaggaatgaggttggttgcgtgtccatatggaacagtctgtaagatgatgcccagcaaaaaggggtacagggtactgccatgtgtgtcatggagaagggaaaatggaaacatccactcccgggaggttctgagaaatgcacagaagcagctgcctcatgccttttgaaacacatgagtgtgttatcctttgaaaagctaggtctgtgaagtcacagaagaaagatgctcactctgtggctctccctcttcccccggcagacatactttgaagacaaccctagggacctccagctgctgcggcatgacctacctttgcaccccgcagtggtgaagccccacctgggccatgttcctgactacctgggtgagtgtggcctgacagggcaggaggcagcaggctggggaagtggcattaatttctccactgctgggtcagcccctgtgcttggtgctggggatgctcaggcagaatagaacctggagaccctggcagcacgcgggcatgtaaacaggcacacccctgtgtttctaaacttgtttgcttggtcccacgggttagctgttgctgtctccattttagagatgaggaaattgaggtagtgcagggtgggtggcagacccagcatttcaggccaggtcgtctccagagctgggccaaatggccatccatgggtcgaagggagtgaacaggtttgggagagagtcacgggcaggaggcagagagagccacctgtgctgcaaaagactcaagattagcagctgctgaagaggcatctgtggagtctctgggtaagaacagtcagcagggagacagactctgtaaggcctaaaccgacaaggtagcaagagaagagccagtggtggtgcgagggatgcaggggttggcaggcatgaggtcaggaccctgggattggtttctgtagtgcagcccaggctagagctttatgtggccattaatactgggcacctctcctcatctttggcaggctctgggtaatgacttctttcagtgtctcataggaggtgtttttggtaatgagtatgtgtgacttttatgcctaaaatggattgaaggaggagagtggtggagaggaggctgtgggcagcaagtgcaggacccttcccaatgccacagggtctgctcagcctggacctgcagccacccagcgggtgtggtgttgctgctatggaggtgacaaagggtggagatggaatgttccagggcaggaaaagcctgggcactgggaaaggaaggatccagaagagatgggaacatgaaaatgccagagagagcggtgggggccgggttcccatgggacagtgagctggaggagaccccagtccaggtcctggcctgagatgtgaggaggggagttgggagggtgggtagggagggagaaggtaaggctagaactttggcctcaggaacccagtctgctcgtatagcggagtcatttgccaaggtgtggccaggaggtttagaagggccaggagaaggtggaaaggtgtcaggatgtgggatgtttgacatttgaaggggagggcccaggtgtggttggcctgggggagtccatggggtgggcgaggtgaagatagagccaagatcaggtgcagctgggatgcggggccccctgtatcggtagtaatgggccacaggtgaagaaactacctgttgacttttatttcagctgcattttctttctttaaggatgtctgtctttttctttcttgttacatgtttgttgtaacaaatctaaacaatataggagagtgatttaaatagtggaagtctaaggtgctcacattctcctggccctgtgcagatgtggtagtgaatagatgtatgtcataggctgccagttgggtcagaattggagaatttgctgcagaatcagcgggagggcagggatgggagcagtagcggtgagcccactgctcaggcaagcatctcttccagttcctcctgctctccgtggcctggtgcgccctcacaagaagcggaagaagctgtcttcctcttgtaggaaggccaaggtacggctcctggggactgcggacagccccaggactcctcccaacctgctcttttgtcatcaccagaatgtggaggcgccttgccctagggaggggaagagagggtgccctagggaggggaagagggggcaccctagaaccgggccccaaaaatctggtgtgggataggggtacttttgcagccgcctgcaggccctgcttttctttccccagctgcctttccccatttccttatctgcagcaccttctggtcgtgttggccagttgccggcacggctccctttgtgtctttctcagttgggtgggtgggtgggtggattgtctgtcggcctgattcccccaactaacctgtgactttgcctccttagagagcaaagtcccagaacccactgcgcagcttcaagcacaaaggaaagaaattcagacccacagccaagccctcctgaggttgttgggcctctctggagctgagcacattgtggagcacaggcttacacccttcgtggacaggcgaggctctggtgcttactgcacagcctgaacagacagttctggggccggcagtgctgggccctttagctccttggcacttccaagctggcatcttgccccttgacaacagaataaaaattttagctgccccagtttgtgcctccagcatatgaaaaggactatttgaatccccaaaacatcaggagtcgggaaacttcggaagacagctgtgcctggctctgtggctgcatgcagtgcttcacttggccagcagaggtcagctgtgccgagctgccccagccatgagaagagaagcctgcccttgctggcaggtggctatggccggcccagagccttcctgcccagctcctgcagccctgctgcctgggatcaggctgggagatgggccttcctgaccgccagccttcctctccccgagcacacgcacatgtagattcggggggaagctgcctgctcttccttagaggagccggggcagctatctgctggtccctttctgaacaactgttgatgtgtgagctgtgtctgtgtgttatgtgcataagcggtggtgtgacatacacacatgtgtactgtcccttatgccctggcctgagctctccagctgccttctcagcctgaaggctgggcttctctgctggcttggggtcctagattgcatgtcacctgcttaccaggcgtcacaaggccatgctgggggcatgaggaggttggggcagcaggagagtggggagaaactaggagagtgcctgagtattttagaaagaaccaagttttttctcggcaaaagcttatacagagacgaaggagtctgtgtctttggtcatggtaggactgaagctagcaggacccgagatttggggcctccatgatccctgctcctcttctgttaacacccaaggatttccacgaagccagtgtgtatgatgggggcaggacagtggtactttctgggcaggtgtgaactagagctgctaaggagctgcagacgatattcttgcagtttggtggttagcagtattcagaaggacaaagagttaatggaactggagataaagagcaaccatttgagcatctgctgggagacatctgtcaactgcacagaccctatcagtgggcatcgctgccacctcttggaagacaagacagggcagagagtgcctgcagtgctgaggcctggtccttgccctaggttggccttcccacctggcttcatggagtgctgaggctggtcctggggacagtgagtgctctggatgttttagccaagctgtgttctaaagtgatgcacagtctgtctccactatgtttatctctctgaccctgtcacttccaagcacaccctaccaagagcttgtatcccagagccaccctgatggagaggagattggtttccccagtgatttccttctttggggggtggggtagaggaacatggagccagccttatgctgtattcgtgcctggggatagcagggtctgggcccggagcagaggagcttgggtaaagatatggaggctgtttgttcaaagtgtacattccttcctcctaacggcatccctgggggaagctatttctatgttttaggtggggaagatgaggcttagaagttgcctggtgaatgaggttctttgcaagatttgggttctggcctgtccaccctggtggagtagctggtaccacgggggcttttgctgtggggttaggcaccatgtgggcgctctggggccagggcattggaaagaatgggaggattgcttgagcccagaagttcgaggctgtatgatcacgcaactgcactccatcctgggcaacatactgagacactctctctctttttttttgagacagagtctcactttgttgcccaggctggagtgtggtggtgcaatctcagctcactgcaacctctgcctcctgggttcaagcaattcttcccgcctcaacctcctgagtagctgggattacaggtggccgccaccacgcctggctaagttttttatatttttagtagagacagagtttcaccatgttggtcaggctggtcttgaacttctgacctcaggtgatccacccacctcggcctcccaaagtgctgggattacaggcgtgagccaccatgcctggccgtgagactctatctttaaaaaataaagaacaggaaggtccatcttcgtgtcctgagactacagagagaaagtaagtataaatggctcgttcaacaccccacctgggaggcaggtaccatgtgcccatttacgtgtgaacaaacaggcactcagggttggcctcttggacttagtctggccaaagcctgtgccctttgcacaaatgtgcaaatcaggactggggcaggccttggatgagggtatgtgtgctatgggcaaatgaacctagggctgtccagggccaaacagcacagagggcatgtgggcctggaagggaggaaggaggtgtggcacatgctgcgtggaagcctaaggcttcactaaacagcagagaagcttggatggttttcaggctggtgacgccctgggctgaagcaggaaggtcaggagaatgcagtggcctctccactctgggctggcacagttttgcccacatgtatacctgaatgggtgcctggctgtgtggactgtgctatggtctggaatcagatggacaaggcacagtctatgaggcaaggagcagagatggtcagccaatgcagactgctcaatagtcatgttgggagttcagggtactggagggctataaggggccctcacccagttggagagaatgctgccttccttgagaaagtgaggtttatgctgagatgggaagggtgggagggaacagcaatcctagcaggggagacagcatgtgcaaattccctggggtgggagggatccctgcacatttgagggtgaaaagaccagagggttgttaccaaaatatgcaatgggggctgaaaatttgattttttaaaaaaatgtaatagtcacatattaaaaattcaaaggatacagaagatggagggtttttgaaaacaagggcattctttttttttttctttcttttttttttttttttttttgagatggagtcttgttctgtcacccaggctagagtgcagtggcgccatctcggcccagtacaacctccgcctcctgggttcaagcgattctcccgcctcagcctcctgagtagctgggattacaggcacccaccatcatgcctggctaatttttgtatctttgtggagatgggatttcaccatgttggccaggatgaactcctgacctcgggtaatccacccgcctcggcctcccaaagttctgggattacaggcgtgagccaccacacccggccaacagcattctgatgatatagctggtgatagcagatgggaggacagtggtgtgcccagaaacctctggacctggagcacaaaaggctcagggtgcattcccagcccagcggattctctgcctgcatctgctaaggatgaatgactcagctttgggggaattagttgtgagactttggacttcaggggcggggccccaagaggcaatggtgataaagttgaatttgagcagggaaggtgccccgtcagctgtcatccttttccccaggaacatcattatgtaagactcctgccttgtggaacaggctgtgagttgctgctcttccattcctcacagccatgtttacaagggtaaggaagggaaggagtggttcatcattgcagagaggaaggtgccttggccaagcagacctgctctgtgccaggcatgacactgggcaaatgcacagtatttagtttatttatcctgaaatgctttcagaactcaatgcatccagtgtcttgttatccctccagcctatccgcaaacctgctgagatgcagtaggtttggcgtagaatgcactgagggtatctgtggcaacagtgggctaaagaacaaggcacatcaagggggtcttccgacgaacaccccaagggttgcctccaccacgcagcatcctgctgtggctcgcctcaatgtcccaggtgctctgtgggcacagtggccaggtcagaccatgatggccactttctgactgtatggtctcatacagggaaggcatgtcacttttcttggccttctctaggttctcacctgtaagtgggggtaaaatgtcccctccaagggttcctgtaggggccgaagtggctcaggcacctggcacgcctgttgcagcccagctctgtgctagcacctcccaatgcctgttgaatccaccattgccttctgggacgtgtctccatacttccacgagaatacgtctagggcacagcctggggttctgcatttgagttctcacctccgtcccaggtgagcccaaaggtgctggtctctagtccacatttgagaggcaaggctttaatatccaccacacacatttattttgcagattggcaaaagcttagattaactagccagcccaatgtcacttggctaagtggtagaggggaggtgctgtgtctgtcagactctagtctggaattggaggtgggatacctaggttcaagtcctggatatgaaacttccctgtcacatcccttctctgagcccaacacttaatccgaaagtcatggtgacgtgggaggccaggtaaagtaggagatgtctagctagattggaatttcaaataatgaggaattttacagcataaccgtgtcctgtccaatatttgggacatatgctaaaacagatattcactgtttctctgaaattccagtatagctgggcatcctgctttttatttgctaaatgtgcaaccctaggttgtgagacctctctgacccacgcgtggcctcctccaggaagtcttcctgagcgccagcctgggctgggcatcctcctctgtgtgtctgatgctgctctgacctcatgaagctctaattgctggggcctgtccccaccttgatgtgggagctcttcggaggcaaggaccatgccaagttgcatatctgtgtgttcctgagtccagtggttcattaaaagcttttccctgagagtatccttaatgccccggaggtaattctcttttcacaacctttctactgcctgaggctcttgaggactaattccagttaaaagcagagggaaggatgtggtaggaatagcaccgcatggagctggactctgtgccccccgtgcagcaggcaggaccctccctctgtgtcacctccatgactcagggctcagacaggaagccctcatcctcgtcctccacgggtctcatgtttgtcaaggccaggggtatcaggcgtagtaggcaggggcccatgcctgctcccatctgggagaccctgcttcaggcccctctcatggccccttttgttccccacagctcactgtacaacttgttggttcaaggttagaaaatgcagttttgtgttgagggggaccatcacagaagacaaagggtccaggatgaaggctacccatcagcttgcagggctgggacgaatgtgagaagcaactgatgcttgtacagtagagagtaagcatgtagggccagtccccagaccttgcctcccctcagccttgacatgtgatctccatttctggtggctacccctgctagtggtctggcttcaccatcttagccctgcccaggctaagaccctcttccatcagaacctgcagctgggatgctgggagcaacagtcagggcagagctgcccatcctcccattcagggagccctcaggaagtacttgggaccccccgaccctttatagattcagcctgcctcatcccctccatggaccaacacgcccttctcctcagcagtgggctgggggaccaggctcctgaactgcttgtggctgttccagcagtggggagatggagggtcacacagtcctgagtctatggctttgacagcaacgggtcctgactgcagctgtattcgtgaagcgaagtacctaatacaatcaccgaaatgtacaaattggacccctataggttcaaggattcttggtgtaggaggtatggcccccgccccgggaaccaggacctcagcttttagaagcaaaatgcatgaatgcagtgatggttaggccaagtgccaagggagacagccaacccctgctatcatggccagaggagggagagaccactcaggcctgggtggtggtagaaatcctcactgccaaggagattgtatgcgcaggcgttgggaaggcccggagaagcctgagagacaggcttggcttggttatagcagagctgggtggagggagcagattagttggcttagcacaggcttcctgcagggtggtggttcttggccagatttgccccagtggggcttttgggaaagtatagaaatattgttgtttttttttttttgagacagagtcttgctccgttgcccaggctggagtgcagtggtgtgttcttggctcactacaacctccatccacctcccgggttcaagtgattctcctgcctcagcctgccaagtagttgggactacaggcatgtgccaccacacccagctaatctttgtatttttaggagatggggtttcaccatgttggccaggctggtcatgaactcctaacctcaagtgatctgcccgcctcagcctcccaaagtgctgggattacaggtgtgagccactgtgccagccaagaaacattttaggttatcatatctgggcaatggatgctactggctttaggtgggaagaggccggggatactgttaaaatcatgcaatgtacaaggcagcccccacaagagagttctgtggttgaaaatgtccgtagtgttgaggttgaggactctgctgtggggcaacagtaggagaaggggtgctaatagtcaggtggtggacagcagggaattacaggtacatcagttaggagtgtatacagctgcaagtaagagaccaccagatggcaggaacagtgggaacagaatggtttatctttttcatgtgtcaagataagtggtgctaaagtcaggcatggtggccctcacttataatcccagcaattcaggaggctgaagagtgaggatcacttgaggccaggagttcaagaccagcctgggcaacacagtgaaatgccatctctaaaaataaaaattaaaaaaattagccaggggctgggtgcagtggctcacgcctgtaatcccagcactttgggaggctaaggcgggtggatcacctgaggtcaggagttcgagaccagcctggccaacatggtgaaaccctgtctctactaaaaatataaaattagctgggcatggtggcacacctgtaattctacttactcgggaggctgaggcaagggaatcacttagaactggggaggcgtaagttgcagtgagctgagtcacgccattgcactccagcctgggcgacagatcaagaccctgtcaaggaaaggaaaggagaggggaggggaggggaggggggaggggggaggggggaggagaaggagggagggggaagattagctaggcatggtgatgagcacctgtagtccctcctagctactccgaaggctacggtgggagaactgcttgagcctgggaggtcaaggctgcagttagtgatgatcgtgtcactgcactccagcctgggtgaaaaagtgagaccctgtctcaaaaagaaaaaagacagataggaagaaagaaagaagaaaagaaagaaaaagagagagagagagagagggagggagggagggaaaggaaaggaaggaaggaaatgcatctgatttttgtgtattgattttgtatcctataattttgccaagttcatttattagttctagtaattttttttattaaaaaaaattttcaagatagggtctcaccctgttgtctaggctggagtgcagtggcacggttatagctcactgcagtctccattgccaggactcaaacagtcctcctgcctcagcctcctgaatagctgggactacaggcatgccagcatgcctggctaattattttattttttgtagagatggggtctcacgttgttgcccaggctggtcttaaactcctgggctcaagtgattgtcctgcctcagtctcccaaagtgctgggattataggcatgctccaccacactcagacaagttataatacattttcagtggcgtatttactgttttagaatataaaatctatctgcaaatagagataagtttaatttttttctaatttggacactcttttccttcctccctccccttcccctttcccttccccttcccttttccctccctccctctctccctctctttctctctctctctttctctcttttctcttttcttttcatttcatttttgccaaattgctctggttagaacttttaacactatgttgaatagaagtggtgacagtatctcattcctagacacttttcgaaagaagacatacatgcaaccaacaaacatttgaaaaaaaactcaatatcactgatcattaaagaaacagaaatcaaaaccacaatgagataccatctcacatcagccagaatggctattattaaaaagtaaaaaaaagaaaaaataacatgctggcaaggtcatagagaaaagggaacacttgtacagtgttggtgggagtgtaaattaggtcaaccattgtggaaagcagcgtggcaattcctcagagacctaaaggcagaactaccattcgacccagcaatcccattactgggtatatacccaaaggaatgtaagttgttctgccataaggacacatgcacacgtctgttcattgcagcactattcacaatagtaaagacatggaatcaacctaaatgcccatcaatgacagattggataaagaaaatgtacatatatgtcatggaatactattcagacataaaaaaagacatgtgattatgtcctttgcaggaacatggatggagctggaggccattatccttagcaaactaacgcaggaacagaaaacaaaataccacatattctcacttatattctctcactaaatgatgagaactcataggcactaagaggggaacgacagatgctggaacccagtggagggtgggaggagggagaggagcaggaaaaataactattgggtactagacttagtacctgggcgagaaaataatctgtacaacaaacccccgtgacacaagtttacctatataacaaacctgcatatgtacaacttaatgtaaaataaaagttaaaaaacaaggccaggcatggtagctcatgcctgtaatcccagccttttgggaggccgaggtgggcggatcacttgaggtcaggagtttgagaccagcctgcccaacttggtgaaaccctgtctctaccaaaaaaggaaaaaaaaaaaaaaagccaggtgtggtggtccatgcctgtaatcccagctactcaggaggctgaggcaggagaattgcttgaacttgggaggcagagtttgcagtaggctgagatcgctccactgcactccagtttgggtgacaaagcgagactctgtctcaaaaaaacaaaaaacaaagttaaaaaacaaaacatcggacaccacacaccacatggcaggatccaggatccaatcagatcaagctctggcatcaccccacggcaggatccagtcagatattaccttccagcatcacctcattgtgagatccaattagatcatgcctcattattaccctgtgcttataaaacccaacccaacccctagctcaggaaaagagattgagcattccctccttccttgccagttgactttaaataaagcttttcttatctcaaaatataaaaaagaaagtatctcccctgggcatggtgggctcgtgccggtaatcccagcactctgagaggcagaagtgggcagatcaactgaggtcaggagttcaagaccagcctggccaacatagcaaaaccctgtctctactaaaaatacaaaaattagccaggtgtggtgcctggctaatttccacgcccggctaatttttgcatttttagtagagacggggttttgccatgttggttaggctggccttgaacttctgaccttgtgatccacccacctcagcttcccaaagtgctagcattacaggcatgagccaccacccccagccctcttctgcctgatcttagaggaaaaaccttcagtctttcatcattaaaaaaaaaaattatttttcgagacagagtcttcctctgttttccaggctggagtgcagtgatgtaatcgtggttcacagcagcctcaaactcctgggctcaagtgatcctcctgtctgagcctcctgagtaactaggactacaggcatgcaccactacaccaagattttttttggtagggtcttgctttgaccttcctttgaccttgctttgacccttgatttgaccttgctttgacagtgtcttgtaatgttgcccaggcttctcttgaactcctgggctccagtgattctaccacattggcctcccaaagcagtgggattatgagcatgaatcattgagcctgccagccttctgtcactgaggatgatataaactgtggggttttttggttgtttttgtttttgagacgaaatctcactctgtcgcccaggctggagtgcaatggcacaatctcagctgactgcaacctctgcctcctgagttcaagtgattctcctgtctcagcctcccgagtagatgggattacaggcgtgtgcaaccacgcctggctaattttttgtatttttagtagagatggggtatcaccatgttggccaggctggtctcttaactcctgacctcaagtgatctacccgcctcagcctcccaaagtgctgagattacaggcatgagccaccacacctggacattttttttcatacatggcctttatcatgttgagagagttacctgtattccttgttttctgagtggttttattatgaaaggatgtcggatattgtcagatgtcttttctgcatcggttgagagaatcatgtgattttttcccttcatcctgttaatctggtatagttcattaattgatttccatatgttgaaccatccttatattccaggaataaagtctacctggtcatgatgtatactctttttttgttttgtttttttttggagagggagtcttgctctgtggcccaggctggagtccagttgcatgatctcagctcattgcaacctctgcctcccaggtccaagtgattcttctgcctcagcctcctaagtagctgggactacaggcatgtaccaccacagccggctagtttttgtatttttagtagagacgaggtttcaccatgttggccaggctggtctcgaactcctgacctcaagtgatctgcctgcctcggcctcccaaagtactgggattacaggcttgagccactgcgcctggccaatgtgtataatctttttaatacgatgttcagcttggttcgctagtacttttactcagtattcatgtatattttattcaatatttatgagatctgtagttttcttgtagtgcctttggttttgatatcagtataccataggatcaggatactatgaacatgccctcatagaataagttaggaagtgttctttcctcttcaatttagggaagaatttgaggaggattgatattatttctttttctttttctttttctttcttttttttttttgagatggagttttgctcttgttgcccaggctggagtgcaatagcgtgatcttggctcacagcaacctctgccaactgggttcaagcgattctcctgccccagcttcctgagtagctgggattacagacatgtgccaccatgcccagctaattttgtatttttagtagagacggggtttctccatgttggtcaggctggtctcaaattccgacctcaggtgatccgcctgcctcagcctcctaaagtgctgggattacaggcgttgagccaccatgcccagctgatattaattctgctttaaatgtttgctagaattcgccagtgaagccatctgatcctgggcttttcttttgggggagtttaaaaattactgattcaatttccttactagttatatgtctatttagattttctgcttcttcatgaatcagttttggtatgcaatgtctagcaatttgtccatttcttctagattatcgtttgttatacagtcatttatagtattatattgtattttttatttctgtaaaattgtaaagttcccactttcatttgtgattttggtaatatgagtcttctctttttcttagtcaccttacctaaaggtttgtcaattttgttgatttttttcttttaaaatttatttttctgtattattttatttgtattatagattgctccttcagaatatttttttcaagaaatcaactttttgtttcatagctgttctctatagttttctattccctatttcacttatctcagttgtagtctttattatttttaaaaattctagctttgagtttagtttttctttttctagctccttaaggtgtgcagttagaaaatttcaaatctttcttcttctcttctcctccccctcctccttcttcctcctcgcccttcctcctcctccccctcctcctgttggggtgatcagacccaacaccaggtcgtgggggtgacaaagtccggtggagtcaaaggattgagacaaagacagtttgagagataaaggtgggacaccaaggggccatcgtgatcatggaggctgcgaaagccctgcgctctgggagtccacagtatttattggtaatccaacaaagaaacaggtggtgaggcatgttctcactcataggtgggaattgaacaatgagaacacttggacacaggaaggggaacatcacacaccggggcccgttgtggcgtgaggggagggatagcattaggagatatacttaatgtaaatgacgacttaatgggtgcagcacaccaacatggcacatgtatacatatgtaacaaacctgcacgttgtgcacatgtaccctagaacttaaagtattaaaaaaaaaaaaaagaaacaggtggtgagaatgtggaggtcaaaagggcaggcgcatgatctacagctgtgacagtttagcatttatatggaacatgttctgctacttgagataatgggaataggagcctaggagggctagaagcaaggagccagcaagtctagacacattccagaggacattatgcaagtcctgcctcagtttccctcccaacactcagctttttcccaacatcctcctcctccttcttctttttttctttctcttcctcttcctttctttcctccttctctcttttttgtagagatggggttttgctatgttaatccaggttggtcttgaactcctggcctaatatgattctcctgccatggactcccaaagtgttgagattacaggcatgagccaccacacctggccctttctttaaaaaaattttttttttttaatttttaaaaatttttttgagacaaggtcttgctctgctgcccaagctggagtacagtgctgtaatctcagctcaccgtagcctcgacctcttgggctcaagtgattctcatccctcagccttccaagttactgggactacaggcacgtgccaccatgcctggcgaatttttcctatctttcttgtagagacagggtttagccatgttgcccgagctggtctccctcaatcctgcccccttggcctcccaaagcactgggactacaggcatgatccaccgcgccagggtgctttcttcttttttgattgtgtttatggctaaaaattttcctcttagcacagctttgctgcatcccataagttttggtatgttgtgttttcattttcatttgtctcaaggtatttttatatttcctttgtgatttttgctttgatccattggttgttaagcatgtgttgtttaatttccaaatatcatgaattttcagggtttttttcctgtaatttatttactttttttttttttgagccaggatggagtgcagtggtgtgatcatggctcactgaagcactgatctcctgggttcaagtagttctctcgcctcagccttctgagtagctggtaccataggtgtgtgccaccatgcctggctaatttttgttttgaaacagggtctcactctgtggcccaggctggagtgcagtggtgcgatcatggctcactgcagccttgatctcctaggttcaggtgatcctcccacctcagcctcctgggaagctgggactacaggtgcacaccactacaccagctaattttttgtatttttagtagggatgggatgtcaccatgttgcccaggaagttctgaactcttgggctcaagcagttcatttccctcagcctcccaaagtattgggattacaggtgtgagccaccacacccaacttatttttatttttagagatggggtttctctatgtttcccaggctgatcttgaacccctgggcccaagagatcctcccaacttctcctcccaaagtgctgtgattacaggtgtgagtcaccgtgctcagccccttctattatcgagttctagtttcattccattgtgactggaaaatatactttctatgattttaattatttaaaatataacaaggcttgttttgtcgcctaacgtactgtctgtcctgaggaatattccatatgcacttgaaagaaatgtgtatcctgctgttatggagtggaatgttgtatatatacaagtgtccaagtgttttataaatgttcaagacttctatttccttactggtcttgtggctagttgttccatcaattattgaaaatggagtattgaagtctccaactacttattgttgcattgtctatttctcctttcaatgatgtaatgtttgctttacatattttaaggtcacattgtttggtgcatatatattattacttattcttgatgaattgacccttttagtaatgtataatgtcatttttgtcttttgtaacaattctttatttaaattctattttgtggtcaggtgcaatggctcatgcctgtaatcccagcactttgggaagctgaggtgggcagatcacttgaggtcaggagttcaagaccagtctgtccaacatggcaaaaccccgtctctactaaaaatacaaaaaattagctgggtgtggtgggacacgcctgtaatcccagctgcttgggaggctgaggcacaagaatagcttgaacccgggagacagaggttgcagtgagccaagattgtggcactgcactccagcctggacaacagtgagaccctgtctccaaaaataaataaaataaaaattctattttgtcagatattagtgtagcaactccagctctcttttggtgactatttgcgtggaatatctttttctattcttttattttcaaactatttgtgtcctcagatctaaagtgagtgtcttagacatcatatagttggatcctatctctaaaacaatgtattctgcattctccaactttgactacagagttgaatccatttaaatttgaagtaattactgataaggatttatgccattttaccttttcttttctgtatgtctcatagatttttgtctttcattttcttcattattgacttctgtatttatttatttatttgctttttgttttattttattaattttttgtagagacagaatctcactatgttgcccaggctggtcttgaactcctggcctcaaatgatcctcctgcctcagcctcccaaagtgctgggattatagacatgagtcaccttgcttggctgggttttaaaaattgtttttgtagtgacacattttgattctcttttcatttccttttgcatatattctatgtattattcttcgttgttaccctggggattacaaataaaatcctagagttataaaaatctaatttgaattgataccaacttaacagcatacaaaactctactcctatacagctttgtccctgctttaggttattggtgtcaaaaattccatctttacacattgtttgctcaaaaatatagaattatgtttttttggctgggtgtggtggttcacacatgcaatctcagtgctttggaaggttgaggtgggaggattgctcgaggccaggagtttgagaccagcctgggcaacataacaagatcccagctttacaaaaaagggaaaaagaaagagtgacttggcaggcatggtggcttagacctgtaatgccagtactttgaaagtctgaggtgggagaattgcttgcctccaggagtttgggaccagcctgggcaacacagtgagaccccacctctacaaaaaatacaagattttgccaagcgtggtggcatgtgcctgtaatgggattccagctatttgggaggctgaaatgggaggatcagttgagcccagaggtcgaggctgcagtgagctgtgattgcactaatgcactccagtctgtctcaaaacaaacaaaaaacaccccaaaaaaaccccaaagttaaaataattctggcttttatatttacctatgtaaatacctttattgaggatttttatttcttcaaacatctttgagttactgtctagcatcctttaatttcaacctgaaagagtccccttagcatttcttataaggcgggtctagtggtaatgaactctctcagctattatgtatctgaaaatgtcttaatttctcacttatttttgaaggatagttttgctgaaataggatttttggttgataattttgtttcagcgctttaaatatatcatgctcactgccttttgacctccaatgtttcttatgagtaatcagctataatcagctgataatcttattgaggacaccttgtatgtgatgagtcacttttctcttgttttcaatattctctcttagtatttgcctttcaactgtttgattataatatggctcaatataagtctctttgtatttatattccttggcgtttattggacttttcagatatttaatattcatgtctttcatcaaatttggaaagttttaggccattatttcttcaaataatctgtctcattctccctttcttctccttattgaactcccataacacccacgttattttgtttcatggtgtaccataagtagcagtctctgttcacttttcctcactctttttcatgtctgttcctcagacctgatgatttcaattgtcctaccttcaggttcacagattctttcttctgctttctcaaatctgctcttgagcccctctagtgaagttttttatttcagttattatccttttcaggtccagaatttctgtgtggttcttttttataatttctctttattgatatcctcattttgttcatgcatagttttcctaatttactttagtcctcatccatttttgctcttagctctttaagatagctattttaaagttttttgtctaataagtgtaatgttgggctgccttggacacagtttttgtcaacttttttttttttttcctttgaataggccatcttttcccatttgtctgacttgtgattttgctgttgctgttgaaaactggacatttgactattataatgtgataagtctggaaatcagattctctctcttcctcagcatttttttttaatttctgaagactgtagtaatgtttgtttttatactttcccaagctatttttgcaaagactattcattgtttttttgtggtcaccaaagtgtctgtttcttcagcttgtgtttagccagtgttttgacagagatttccttgaatgccaggagctaaaaaacaacaccaacacacacacacacacacacacacacacacacacgtacacacacaagcatacctctcctatcttttgcaaattggggttgggactcttttaacacttagctaggcttgttctgagcctaggatcagcctgcgacaaaagtttcagggcttttctgaacatgtgttttgccttgtacatgcatgcggcattctcaatttcctgtatacatagccgttttatttttgtttgagttggatctcactctgtcgtctaggctggtgtgcagtgacatgatcatggctcactgcagccttgaactcctgggctcaggtgatcttcttgtttctgtttctcgagtggctgggactacaggaatgcaccaccatgcccagctaagtttcccttcccttcccttttctcctctccccttcctttcccttcctcctttcttttctcttttcttttctctctctttctttccttttctttcttctttctttctttcctttctttttctttctttctttctttctttctttctctctctctcctccctcccttccttccttctttccttccttcctttccttttttcttttgcttttttctttcctcttctttcttcttttctttctttctttctttctttctttctttctttctttctttctttctttctttctttctttctttctttctctttctctctctctgtctcctccctgccaccctcccttccttccttcctttccttttttcttttgcttttttctttcctcttctttctttctttttctttttctctttctttttctctctctctttccttctctccctccctccttcccttccccctccctccccctctcctcccctcccctccccatcctgtccttgtgtgaacatagctcacagcagccttaaccttgagggctcaagtgatcttcctgtgtctcttccaagtagctgggacagcaggtgcctaacctccgtctaattatttatttttttctgctcatcctctgtgggttggacccactgccgaaccagtcccaatgagatgaactgggtacctcagttggaaatgcagaaatcacccaccttctgcactggtctcactggaagctgcagatgggaactgttcctattcggccatcttggccccttccaattatttatttttttgtagagacagggtctcatcatgtttcccaggctggtttcaaactcctgggatcagggcaggatcttcccacctcaacctcccaaattgctgagattacaggtgtgagccaccatgcccagcctgcttttctattttgttgtagagacaggctctcactaccttggccaggcttgtctcaaactcctggcctcaagcagtcctcttgccttggtcttccaaactgctgtgattacaggcatgagccactgcacctggcggcttcttcttcttctttttttttttcttttgagtcaatgtccagcctggagtgcaatggtgcggtatggcttactgcagcctcaaacccctaaactcagatgatcctcccacctcagcctcccaaatagctgggactacaggtacatgccaccatgccagctaactttttttacattttattttttgtagagatgggggtcttgcaattattgcccaggctggtctcaaactcctggcctcaagtgatcctcccaccttggcctccaaaagcattgggattacaggcatgagccactgtgcttggctcaaagctgctttaaaaatttatgtacatatatatattttaagacagagacttgctctactgcactggctgtagtgcagtggcacaatcatggctcactgcagtctcaaacttctgggctaaagcaatcctcccgcttcagcctcccaagtagctgggactacagttgcatgccaccacccccagctaatttttaaattttttgtagagacagggtcttgctatgttgtccagactggtctcaaactcctgggctcaagcaatctgcctgcttcagcatccgcaagtgttggggttacagatgtaagccactgcgcccacgagttgctgctgaatatccaaattgtctaagcttctcctctgggtttaaaatggtctatggcatgtctctacctataacctcttgccccaggcatcttttctgagcaatgtcctgattttaggtaagagatacagcatcttgcatcagttcttccaggatcccccagacaagaacagatgcacgtaatagtttgcaaataaggcctgctctctttggaggagggagctgagaactgtactactgttgtctcaattccaaaactgttgactgagtgcagtggctcacgcctgtaatcccaacactttgggaggccaaggcaggaggatcacttgaggccaggagtttgagaccagcccagacaacatagtgagaccctatctctacaaacaatttaaaacactagctgggtgtggtggcacatacatgtaattctagcttctcaggagacggaggttggaggattgcttgagcccaggagtttgaggctgcagtaagccatgattgtaccaatacattccagcctgggctacagaatgagaccctgcttcaaaaagaaaaaaaaaaaaaaaagaccaagactgctgccatgctggggaaggggtggggcaagactaagtaaaaacaccacaaaactttgctactgttttgaagatggccttttttaaattgagtgtttgcctggttgctgtaggcctttgttttctagagtgacaacaaagttggttctgacagtttggcttgtttattcagtgtttcagtttggaaatgagagcttggagcttcctaggccaccattttgctgatgtcatttccaatggcattttttgcatctcgactttttcctcgcgttcaatgcttcaggaccacaagatggttgctacagctctagaccttccatctgtctagtgtggcgaaaagtggggaaggctagaatatcatgccagctgcatacctcccctttcatgagggaagaaaaagccttcccacggggatcacagggcccctgctagctgcaaaggggtctgggagaacagggagagcctctctcacctgagcagtggacacaatccttcaccaaagagtgcaggttctgatggcaagaaagacaaaggggccaccggcaggctcgttaccccaaagagcgagaagtaggggatgtgattacttacatctgtaccagttagagtgttgtacacatatccagccaaggtacctgtggcccaggtcaggtgactggcttagcaatttcacctaccttcctctcagcccagatccccaaattctttgaatgctgttgggatgcagaacagcaagtcagcgagtgattttttttaatttaatttttatgagtacacagtagattatatatttatggggtacatcagatattttgatacagatatacaatgtgtcataatcacatcaggttgtaaatggagtgaccgtcacctcaagcatttgtcacttctctgttacaaacattttaattacacccttttagttattttaaaatgtactgctgattgtaattaccctgttatgctatcaaataccagctcttattcattctatctaattatatttttgtacccaccaaccatctccgcttccccctacctccccactactcttcccagcctctggtaaccatcgttctacctactgtctattgccatgcgtttgttttcatttttagctcctataaatgagtgaaaacacatgaagtttgtctttctgtgcctggcttatttcagtgagtgatcctcatgtctccagggcttgtctgtacatgactcacctggggcagcctctgccaggtgtcaccccggagccagcaacaaagggctgctctgctgatggctgcctcacccccggctgctccctcagtgaactggcacagctctgggcccctctcgggaccttctcagagtagccacatttcagacctgtcttatgattctaacatcaaacttataatatcaatcttactaataccaatagaaagtggaaaatgaggtattatctggcagtcattaaattagtaagttctaatgacaaacataatacacgatgaaggtgagactgtgggaagatggtgcctttgcgagttgcccatgtcagtggtaagagtcacggccctcgggaaatcaccgagtcttcattacccaagactggcatcaacccttcaccaaattccaataactgagaatctgataattacccaataaatcctagattagcctgaggaaagaaatgagctgtccacgtaagagtcgtaaacattgggccggggctggtggctcatgcctgtaatcccagcactttgggaggccgaggcaggcggatcacgagatcaggagatcgagaccatcctggctaacatggtcaaaccctgtctctactaaaaatacaaaaatgaaccaggcatggtggcacatgcctgtagtcccagctactcgggaggctgaggcaggagaatcatttgaacccaagaggcagaagttgcagtgagccgagattgcgccactgcactccagcctggcaacagagtgagactctgtctcaaaaaaaaaaaaaaaaaaaaaagaatggtaaacattgtactctgactcacaaatctcatctaggggaacttgttttaaggaaataaattcaaagaaggaggaaacattgttaggtgcaaagaagtcaaccagaaacttatttatcaaaaatgaactattgggaaccggctcgacagtcagcaccagaagaggagaagatccacgcgttctgtggaccataacctagtcacggacgtgctgatcagagattgaaggcaacagggaggatttatgtgaaaagtcaagagaaaaagcaggatgcatgtacatatcatatggttacagctcggcacgtgtgtccagaggcaccggcagctgggttgggagatcgggtgtgaaattttcactgtcattccgagccggattgtgccgctgttatgctgcgtgtgtttcacaaatgaccccaggagaccacatagctggactctatctctctgtggtgctagactgggcacagctgggctccaggggcttagcctagacagcccccatgggaagaaacatatgaaaggcagggtgggcctttcatatctttgttctgacacagctctgtgcatgccgacagtgtcttcttgtcgcaagtgcccacggccctgcctaaggccctttgacactgaaggtgcccgccacgtgctggggcgaaatcttccaggaatgtcctctaccagtgacagatgaatgtggtggaaagctgtctgtgtccttattccttggaggggaccttcttgggcacgtccccaccagttcccggaggtccctgggggcaggagcaagctcttggatgcattctggtcagctttcttccatcccctggctcattccccattcaccgactgctgtcatctggggtcatctccccaataaactctttgcactgggatccttgtttcaggatctgtttctggaggaactagatgacaacaccgggaacagaggacctagagaggcagcttcatgggtggtggggtgtccgcctctgccggccagggacttgggagcagtgctgggaaggtgctggatggagctgtcactcacaggggcaggtccttggctgctgactgtcttcctctccactatggctgtcttgagaacttaggggtcagcctgaccctgccttggcccccttcctctcagcctctgtcttctcctgcatgaggctgggtggctcccctgtgaatcaggcaggggtccacagaacactagagacaggtcccttcctgcagctgtctccagtaggtggccacgcaggagatgttcccaacaagctgcccttatctgcagctcagctttggtaatgggggcccattaccaaatgggggtaaaggtcatggcccatcctggtgatagtgagaacccaaggtaggccttgaagattcctatcaggagggagcagaaagtgtgtaccacacccctgggcccaggtggagcagggctgctgctcaaggctcccagccatgctctgtcccttgctaggggtgaccggtgggacaggcctgggcaagggacaagagggagaaggtcggggggaagaggggatgaagagcaaagtgagcaaaggagagtcttccactatctggggtctctgtcaactgtcaggccctagagtgagctgttctttccctttgcttcctggaggaggggacttttgtcactgcgtcactccaccctgcctgcccctccgttatcaggctgttaatattaattaacaacagttgctagggatgacagtgcagagggttcctctgagcccattgctggccctggtcccaagagggggtagggcagagctggggtctgaggctgagccagggagggtgcggaggttcctcggccatgctgagctcctgaggccgggtcccagccagtgcctggtcccatctgtgcctccaggccctggcaccaactccagcagtgttaggggctaatagcgtggtctctcccctagctgactcagccctctggcttcggtcgctttgggaagtgagtggagaccctagcacctgcgtgatgaggctcatctaaagcgggggcctgtggactggggccaaacagtgggagtggtggatcattaaccagcagggctcagcctcattggtccctaacccagtcaggccagggttgtcatcgaaggggaggaggctgccttaatgtgtgttcagcccttggctgttcctgaggcctggcctggctccccgctgaccccttcccagacctgggatggcggaggccggcctgaggggctggctgctgtgggccctgctcctgcgcttggtgagtcccagggcttggctccacctcccctgcggcctccagttagggaccctggggccagccgtgtaccaggcgagcgttactgggtgacagcaagggagcctcagggcctgcgggctgggcaagtctctggacacatgagggatgccaggccccacagaggaggggtgcaggtggagggtttccaggttacaggcttgaatgcacacaggggtgaaagaggctgctggactggggtgctccaagtccctcctgtcactggccctactgtggggtccaggcctgcagttgagggaggtctgaggcaaggaggtgctgggatggggttacctggtgagcatcacctagggaggactgagcactctggaggctgggagaagatccagcgctggcacctcttaagttcctcgcttactttgtgtctgggaggtgggtgacagcttttggcctcaagcaggtggtggtagtggtggtgggagtcggggggcctcctgaacagactctccatgagagaccctggcctctggatgtggtgtacagtgtggggactcaggctgactttgacgtgggcagagcccgggaccttggagtcagctttgcctccttacccatctctggcctctccagcatgactttcctaagctgcaggtctatcaggccacccccaggaagaaaggccagtgttgtcactccaacactggctggctggcacatgcctccaggaggcttcctactccccacactccccgcttccctgcccctgctccatgtccttcttaccctcacaccctccctggctgcctgctgcctggatggcacccagctgtgtcagggcccacgcgtgatgttgctgtgctctgcaggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaagtgagtccatggtggggcccaagcgaggagtgggctggggctggggctgggctgccatggcctcctgggaacctggccgggcatacagctggtcctgaaggaccagaggtagctattcctacggctctggcctggggccgcccagatgattatctctgcccctcgtccggccgccatttcctttggtcagagttcctgctcatggctgcaggtttgtgcgtggccatcgctggcccttcaaccccgagtccactctgtctttctgcagatttcttgacatgtgggagctccctgccacactcttgctttaagtctgacagaggagcccgattggcagagtacatatttatatttgctatgttttgcttcttgtttctgtgccaggggccgtagggccatcagtaacccatgaggtaccatggtatgcattggaaaaggtgccctcaggccagaggtcgtggctggtctcaggcacctgggccgggtgtcctggggtaggccacagccacacacacttctattgattggggttcggtctttggttctgtccactctggtgtgctgccaacaagatgccaacaacgctgctgggccaagggggccaagagccaagggcagcagcagggccttggcagtggaggctccttgaggttggagtagagcagaggtcctcaagatgaacgtttagtactccatactccagagcaaatgagagttaaaaggggcaaatagcatcttagtgttattatgaaaacagttctgaccttacagaccctggaaagggtctccaggacgcctaagggccccaggccacactttgagaaccactggattggaagagagtgccgacactttctgtcccctgctacctggctctgcatccctcagctgggccccaagtttgggctgcttcccagagtgtctgtgccaggaacccaagggctctctcttggaaatagcaggaacgagaggagccattgtttgctctggggaggcatcatggtctgacctcagactcatgtctgacggtagctttatagtccattatagggtattatctttattttgacttcggatgctcacaacaactctcgggtggtccaattatctccattttacagacaggaaaactgaggttcagaggggtgtggtaagctgctcaaggtcacacagcaaccagcactcgcttgctgagatctgagagaggggggtagagagctttgctcaggtgtcccactgcatcttcgcaatgacgggctttgcagaaagggctaagctgaaggacctacagacttgcctgagggcaccagtctagtaaactgtgaaaacattggctgctgggctccagggttccaaatctaacctcaatacctaaagggtttcgggggccctaggcaggagaaggaggctgagagggcaacgtttgagacagcccatgccagaccccatggctcaaatcccagctcttccaccctcacgggacttcaggtgtgacgctcaatccagagtcagataatgtcagagccaggaaggtcaggccagtgtgtggagacatgagaggctcagagggacaggtcccggagcagcccctgcctgccacagagaaggcactcagggcagctccaactcactccgtgggtgggggcctgcaggagatcttgctggatgggagccatttaggacccactcggctgggtcctaaatagctaaatggcctaaatgcagatagctgggctatctgcagccagtgtcccccaccccaccagctcaccctccatagtgctgtgggtctggggtgggaggggaagggaggggccatagggactgggcagggccaggaaaggccctttccctttgcggtcatctccctctagtgccccgctcaccttcaaggatggcacagccctggccctgagctgcatggctgactacggggcccctgtcttccccttccttgccattggggggtacaaaggtaagctaagtgggccctgagaggaagccaaggaagatgcagtattggggcaggaaccatagacgggagggtgggagtggtgctggggattctcgcggcctgggggtagcctggcttctggaagctgtaggccaaccctgtcctgtttcctctctctgccatctcctttatcttctagtagtgttactcaggcactgtggtttttctgcctgggcccaaaggtctcgcctttggctgagagaagtggggtgtaggaggtaaggccatgtatcagatgaggaaggagtgggggagaaggagcaaggggtgatgggaggggtgcagctagatagggggagggaatataggggtgcagctggagggggagggaggcacgggtgcagcaggaagggtctgagtatttcttatcccaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgaggtaggggctgcagggtccctggctctgggggtgcaacccaggtggtcttgggtcagttcctgtgtccccatcctggccctggcccttcctaagtgaccctgggcagtggctgcctgctcagaacggggtgattgtgatggctgttcttatagcctcacctgcgattatagggggccatcaggccctatgacacaacacacaattagtgcccagtgaccgagctattgagagctggcctggctgaagcaggcacggtcagtgggggctggtcgggtgtgtgtccacagcgctctctggaagacgagatcaatcgcaccacagctgaagacctgcccatctttgccaccagctacattgtcatattcctgtacatctctctggccctgggcagctattccagctggagccgagtgatggtgagaagcgggagggacacagctaagtgggctagcccaggaccccaggcatcttcagtaggccttctacaactttcctaaccacagcacctcagaacagcaaagtggacacacccaagtggctgccccaaagggtaatacctcttgcaagtgttctgtgctgaaaggtcaagagcaattttcttttcttttcctttctttttcttctcttttctttgcttttcttttctcttctcttttccctcctaccctctctttctctttcttttctttctctctctgtttctctttttctctctttctttcttttgagacagggtcttgctctgttgcccaggctggagtgcagtggcatgatcttagctcactgcaacctcaaaactcctgggcacaagtgatcctcctgcttcagcctcccaagtagttgggactataggcacttgccattgtgcccagctattttttttttttttttgagacagagctttgctcttgttgcccaggctgcagtgtaacggcgcgatctcggctcactgcaacctccgcctcctgggttcaacaattgtcctgcctcagcctcccgagtagctggcattacaggcatgtgtcaccacgcctggctaattttgtgtttttagtagagatggggtttctccatgttggtcagactggtcttgaactcctggcctcaggtgatccgcccacccaaagtgctgggattacatgcgtgagctaccacgtccggccattttttttgttttgtagtttttgtagagatggggtctcgctttttgcctaggctggtctcaaactcctgggctcaagtgattcttcctcatcagcctcccaaaatgttgagattacaggtgtgagccagcacacctggcctaagagcagttttctgtctgttacatgccataccctcacttgcccaaatgcaaagctaagacttaaaatctcttgcaatgcatgctcaaggaagatggagtaggctcacccatgcctttgggtttcctggacctccccttgggaggatggctctgcagaggggctttaatgtgagatgtgagctcctcaccactgggggcagtatcgggcacctgcaggcactgagggtgcctgccggctactttgtctggcctagctgaggctggtgggcatactgggtaggtgctaagtggctagggggctgagcctgtttgcattgcaggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccaggtaagaagggaggagctctccacacccccaactgcccactcttctcccaacctcacctcctggcctgatgggactctggcgtgaatttgctgggtctccctgcagactctttctgttcatcgacacgcatgtttacaatatctgtagaaactagagtgtgttgacataaatgacttcatcctgcctctaccatctggaattagctttctgttaaccccttgcaatgtctagtaaaacctctccatgttagtacattacagcctcctcctgtctttatgctgctaggtagcattccatggtaaggataaatcagagtcgatttcacctctccctgttggtgaacaattagggttccaacagtgcttggaacagggatgctatagacatctcaaatgcaccaaccatttctcccagccagaccctggaagaagaatattggccatggagagtatgagagtctctgatgattcaggaaggtcagagcagctcctcaggcctggctgcagctctgggcacttgccaactccctgctggcctttgaggggcggtgcccttggagggccctggctcttatccctgctgttcccacacagaggctgccccggaggcctggggagccacgagaggtccacattgggcgagccctaggcagggtggctcccagcatgctgttgtgcagcctctctgaggccatctgcttcttcctaggtgagcctgggtgagacctccccactcggcattaggcttgctgggttagtgccggggcctaggagttcccagagggcagtgggtatagtgcagattcccttccccctgcaccctgtcaatgtcggctaccactctgcccttgaagccagggtgccctgacagccctctgctccctcacaggggccctgacccccatgccagctgtgcggacctttgccctgacctctggccttgcagtgatccttgacttcctcctgcagatgtcagcctttgtggccctgctctccctggacagcaagaggcaggaggtaggggcagctgggccagtactgagggacctgcccctgggttcccaccatggcagggagatggggtggctttaccaccacagagatggcccagagaatggggtgggggacaggggcattgtgccaggagagtaatatttaggccatgtattctccaatttcctacagaaaaataaatttgttttgacaattttttaaatataatcaaacctcctaaagtgcatgatgttgagaaataaaatacagttgacccttgaacaatgtggagattagggcaccgactgtctaagcagttgaaaatctgcatgtaactttttttttttttgagacggagtttcactctgtcacccaggctggagtgcaatggcgtgatatcagctcaccacaacctctgcctcccgggttcaagcgattctcctgcctcagcctcccaattactgggattacaggccccctcctcctgcacgcctggctaatttttgtgtttttaatagagatggggtttcaccatgttggtcaggttggtctcgaactcctgacctcaggtgatctgcccaccttggcctcccaaagtgctggcgtgagccaccatgcctggtctgcatgtaacatttgacccttctaaacttaattcctactagcctactattgactggaagccttaatgataacataaatagtcgataacacatcttttgaatgttatatgtattataaactgtattcttacaataaaggaagcaagaaaaaagaaaatgttagtaagaaaatcataaggaagagaaaatctatttactattcacgaagtgaaagtggatcatcatgagggtcttcatcctcgtcgtcttcaggttgagtaggctgaggaagaggaggaagaggagggcttgatcttgctgtttcaggggcggcagaggtggaagagaatccagggataagtgagcccaggcagttcaaactcgtgttgttcaagggtcagctgtataaatgagaggtcgacaggagttgatctgttggttcccatgatggtgtaaaatttaaagatattttatcaagattaaaataaaagcaaagaaaacagcacactggtatgtctccatgagggcactggcacgggccacccacagaaggtgacactccctgggggcaagaaggtggtccctggggccttgtctgctctgggactaccttgagggggtgcctcccactccaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtggtgagtgggcctcgaaccacacgagagcaggggcactaggtggggacctcgcctcagggagagcagggttggaggtggggaggttgcctaggcccaaatgctgatacttggggctggcacgcaagtctgctcaactccagaatgttgcccatgacaccctgactgacttaaatttgtggggagatgggggacggctgttgggcagggtggtctcatgcagcaggtcccttctcagctgctgctgtttctcgccctgttcggagtgagcctctactccatgtgccacatcagcgtgggactggaccaggagctggccctgcccaaggtgagcccaggcccttctcaacccttaggcccctgggatttggggaggggcagtagcaaccagcagggatgggttggggggtcctccggccaggggcttggccagaggtgcagaattgttcattactctggaggcacctccagcagtcctggggagtgaagccacattcgtgtatgaacagcacaacagccaggtgccagccccaggccacagtaagagagatggcccaggcatcggagggctgtccatgtgagatggcaggccacaaagaatgactgccactttgctgagtgcctgcccagtgtccagccctgcgaattctctgggcctgaagcccggggagggcaggggttcaggggaaggaaagccccgtggttggaggggacctccaaggtcacataggatttgcagaggaaagtgatgacagactcgccagtgggaggctagggtgagcccaggtgtgtttcctgggcgtggcagcgactgtgggggtgggatgagctggaggccaagggcatggtcggggagagtgctgattgcccagcctggaccagtaagtgtgcgggccaacaggcacaatgcatcagccaaggctggggacccggctcctctggatatgcatcagcggtggccatgggctggtggccaagaggaagcagccacagacaacaaagtctgagacacatggtcagactgcatgagcaagctctagggagagggaaggcatcgaggggactcgatgtctaggtcccatctggggaactgtgatggaggtttgggcaagggtctgggtactggcaggagccccagtggaagcagccaggcctgagcccacaacagggctgagtggggtgcggctggggtaggtgtgttaggcagtactggcctggggtcctggaagccaggtgagggaggacaagagcagatggctcaggactgtactttgggtgactttatggagggagagcaggtgaggagtcacagaatgaacctgccacctgcagaagccctgggggctatgtcacagggctgaggtgaagagggtctctagtgccccaagagcaagaaggaaggatgtgatgggctgccagaccctgctgaggttttatgttgatgtcttttgtttatttttctgttggggacatttgtttcttactgcttttaaaaattttatcattttttttccgttttttattgtggtaaaatacacataatagaaaattaccattataaccatttttaagtgtacagttcagtgatattaagtacactcatactattcaactatcaccaccatccatctccaaaactctttcctttttgcaaaattgaaactttacccaacaaacagtgactccccattctcccctcccctcagcccctgacacaaccaccttttatttatttatttattttgaaacagagtttcactcttgttgcccaggctggagtgcaatggtgtgatctcggctcaccgcaatctccgcctcccgggttcaagtgattctcctgcctcagcctcccaagtaactgggattacaggtggccgccaccacgcccagctaatttttgtatttttagtagagacagggtttcaccatgttggcctggctggtcttgaacttctgacctcaggtgatccaccagccctggcctcccaaagtgctgggattacaggtgtgagccaccgcacctggcctctactttttctttttttttttgagatggagtcttgctctgtcacccaggctggagtgcaatggtgcagactcggcttactgcagcctccacctcccaggttcaagcgattctcctgactcaacctcctgagtagctgggactacagccgtgtgccaccactcccagctaatttttgtacttttagtagagacagggtttcgccatgttggccaggctggtctcgaactctggaccttgtgatctgcctgctttgccctcccaaagtgctgggattacaggcatgaaccactgtgcccggcccatttactttctgttctatgagtttgaccactctaggcacctcaggtaagtgaactcatacaatatttatttttttggctgggagtggtggctcactcctgtaatcccagcactttgggaggctgaggcaggcagatcacctgaggtcaggagtttgagaccagcttgaccaacatggagaaaccccatctctactaaaaatacaaagttaactgggcatggtggcacatgcctgtaatcccagctactcaggaggctgaggcaggagaatcacttgaacctgggaggcagaggttgtggtaaactgagatcacgccattgcactccagcctgggtaacagagtgaggattcgtctcaaaaaaaaaaaaaaagtatattttgtctgatcttagtatagctacccctattctcttttggttactatttacatggaatatcttttttctgttcttccactttcaatctatttgtgtttttggacctaaggtgagtctcttggagacagcatatagttagatcacgttttgctgttttttagcagatgggggctgcctagggcacagtatgctgactctcacaatctcgatcgtgtgtgtgtgtgtgtgtgtgtgtgtgtgtgtgtgtgtgtgtgtgtgtgtgtgtttaattcattctaccactcttttttttcttttttttttttgagatggagcctcagtctgtcacccaggctggagtgcagtggagcgatctcagctcactgcaacttacacctcccgggttcaagcaattctcctgcctcagcctcctgagtagctgggattataggtgcatgccaccatgcctggctaatttttttgtattttttgtagagacagggtttcaccatgttggccaggctggcctcaaactcctgactttgagtaatccacccacctcggcttcccaaagtgctgggattacaggcgtgagccaccatgcctggtcctactactctcttttgattggagagtttaatccatttacatttacagtaattattgataaggagggatttacttctgtcattttgctatttgttttctatatgccttgtagattttttgtttctcatttcctgcattactgacttattttgtgcttagttgattgctactagtgaaattttacattttccttctcattttcttttgtgcatagtctacagctaattttatttgtgattaccatggggattatcttaaatgtgctgaagttataacactctaaatttatgccaactttgtttccatagcatacaaaaactctgccctataacaactccatcttacctccctttcagttattgatgtcacaaaattatatcttgagctagccatggtggcttatgcctgtaatcccaatgctttgagaggtggaggcaagaggattgcttgaggccaggaatttgaggccagcctagccaacacagtgagatcccatctctagaaaaaatttaaaatttagctgggcaagatggcacgtgcctgtagtcccagctatgtgggaggcttgcttgagtccaggaattcaagtatgcagtcagctatgatcatgccactgtactccagcctgagcaacagagagacaccttgtctcaaaaaattttatttttcagctgggtgtagtggctcatggctgcaatcccagcactttgtgaggtggttggatcacttgaggccaggaggtcaagattagcctggccaacatgacaaaaccccatctctactaaaaatacaaaaattagccaggcatggtggcacacaactgtaaacctagctacttgggaggctgagacatgagaattgcttgaatctaggaggtagaggttgcagtgagctgggatcgtaccactgtactccagcttgggcgacagagcgagactatgtctcaaaaacttttgtatttttatgcattatgtatccaaaatcataggctaatgattttttttgcatgagtctcttaaatcatgtacaaaaaggtggagttataaatcataacatttataactgcccatttatttacctttgccagggatttatttatttatttaaagaggcagagtcttgctctgttgcccaggctgagatgcagtggtgtgatcatagctcactataacctcaaactcctggcctcaaaagatcctctcacctcagccacctgaagtactgggattacaggtgtaagccactatgcctagccaagggatttttatttcttcatacatctttgagttactgctgatgtctttttttttttttttttttttttttgagaagttgttttgctcttgttgcccacccaggctggagtgcagtggcatgatctcagctcaccgcaacctctgcctcccgagttcaagcgattctcctgcctcagccccccgagtactgggattacaggcatgtgccaccacgccaggctaattttgtatttttagtagagatggggtttctccatgttggtcaggctggtcttgaactcccaacctcaggtgatctgcccgccttggcctcccaaagtgctgggattacaggcatgagccaccatgcctggcctgtctaatgtctttttatttcaacctacaggattccttttagcatttctttcagggaaggtctagtgataacgaattccttcagcttttgtttatctgagaatgtcttaatttcaccctcattttaattttttaaaattttttatttattttgagatggagtttcactcttgtcgcccaggctggagtacaatggtgtgatctcagctcactgcaacctctgcctcctgggttcaagtgattctcctgcctcagcctcctgagtagctgagattacaggtgcatgccaccatgccaggctaatttttgtatttttaatagagacggggttttaccatgttggccaggctggtcatgaactcctgacctcaggtgatccacccaccttggcctcccaaagtgctaggattacaggtgtgagccactgtgcccggcccatttttattttttaattaaaacaatttttttgagatgggggtctcactgtgttactcaggctggtctcgaacttttgggctcaggtgatcctcgtgtctcagcctcccaaagtattgggattataggacgaatcacctcatctggcatctccctcatttttatttttaatttttagttttttttttttttttttgagatggagtctcactgtcacccagattggagtgcggtggtgtgatctcggctcactgcaacctccacctcccaggttcaagagattctactacctcagcctccaaagtagctgggattacaggtgcatgcctccacgcctggctaatttttgtatttttagcagagatggggtctcaccatgttagtcaagctggtctcaaactcctggcctcaaataatctgtctgcctcggcctcccaaagtgctgggattacaggcatgagccaccatgcctggccttctccctcatttttaagtgacagttttgctggaattaggattcttcattgacaattgttttttcttcagcacttgttttttgttgttgttgtttgtttttgagacagagtctcactctgtcatccaggctggagtgcagtggcatgatctcagctcactgcaacctctgcttctcaggttcaagtgattctcctgcttcatcctcctgagtagctgggattacaggtttgtgccaccatgcctggctaatttttgtattttcagtagagatggggttttgccatgttggccaggctggtctcaaactcctgacctcaggtgatccacctgcctcagcctcctgaagtgctgggattacaggcatgagccatcatgcccagcattcttcagcactttcaatctacaaacccactgccatctgggcttcaaggtttctgatgagaaatatgctgataatctttttgaggatcttttgtatatgccaagtcacttcttttttttcaatattttctattttttaaaaaacttattttattttactttttatttttattttttagaggcagggtcttgctatgttgcctagaatggacttgaaaccctgggctcaagcaatcctcccacctcagcctcttgagtagctgggactacaggtatatgccaccatgcctggcttgtctttggtttttgacagctaaattataatatccagctgggtgcagtggcttatgcctgtaatcccagcactttgggaggccaaggtgggtggatcacaaggtcaagagatcaagaccatcctggccaacatggtgaaaccccatctctactaaaaatacaaaaattagctgggcatggttgtgcgcacttgtagtccaagatacttgggaggctgaggcaggagaatcacttgaacccaggaggcagaggttgcagtgagccgagattgtgccactgcactccagcctggcaacagagcaagactccatctcaaaaaaaaaaaattataatatccgttggtgtgggtttctttagtttatcctattggagtttattgagtttcttgaatgtttatattcatgtctttcatcaaatttggggagttctggccataattttttcaaataatctcacttcccctttctcttttcttctggaattcttacaattcatattttggtctatttgatgatgatgatgtctgacaggtcccttaggctctgctctgttcactttcgttattttttttcctttctcttcttcagactcagtaatttcaatggtcttatcttcagtttgctaattctttcttctgactgcttttgaatccctctagtgaatttttcatttaagttactgtactttttagctccagagtttttttgctcttttttatgtttcctcctcattgatatttccattttgttcataaatttttccttgactttgttttcttttagctctttgagcaactttaaggcaattgttttattcatttattttattatttatttatttattttttgagacagagtcttgctctatcacccaggctggagtgcaatggtgtgatctcgggtcactgcaacctctgcctcctggggttaagcctcagcctcccaagtagctgggattacaggtgcctgccaccatgcttggctaatttttgtatttttagtagagacagggtttcaccatattagccaggctggtctcgaactcctggcctcatgtgatctgcctgccttggccttctgatgttgtgggattacaggcatcagccactgtgcctggctgagacaattgttttgaagtctttgtctagtaagtctgctgtctggtcttacccaggaacagtttctgttggttaatattttccctttgaatgggccatgtttttctttttcttggtgtgtttttggttgaaaaatggacatttgattcttataatgtggtagctctggagatcagattctccttctttcccagggcttgctttattttatttattgctgttggtgtttctgtgctggggatcagccaaaggcacagagttaatgtcttctcaggtatttttgagactgcatttttctctgagcatttatgcagtgtggtgactgtctaaatatccctatatttatggttgcttttgaatgtccttgtccttatatgtatggttcccaaaaggagaaaaagggaaaaatgaaggtgtcggggataggtgcttactctttaaatctcctggaagtcactttagtaagatgtggaggtggttgcaacaacggtggtgggagttgcattagtggctgcctgcctgtgtatctgtaccaccaatatcagaagtaatgatcaattatcagaactcagatccttgatatttgaacttatttatttatttattagagacagggtctggctctcttgctgaggctgaagtgcagtggtgcaatcataggtcactgcagcagcaaacttccaggctcaaatgattctcctatttcagcctcctgagtagctaggactacaggcatgtgccaccacacccagctaacttttgtatttttttttgtagagacagggtgtcgctatgtgcccagatcggtctcccactcttgggctcaagtgaccctcctgcttgccctcccaaagttctgaaattacaagtgtaagccatcatgcccagctgatatttggtggatggtgtccttgcctacctggctcctgcaagctgtgtacaagctgcttctggaaagcatacacagctgcatgccttgaggctgggagtggcaaatgggtagctgctactgtactaaagctgagattgcctgaaattaaccacaatttactgtccaagccttatcctggaagcttccagccctcaatagactccagagttccaaaatcgttacactagggccggtgtggtggctcatgcctgtaatcccagccatttgggaggccgagacgggtggatcacttgaggtcaggagtttgagacaagcctggccaacatggtgaaaccccatctcttttaaaaatacaaaaatcagctgggagcggtggcacatgcctgtaatcctagctactcaggaggctgaggcacaagaatcgcttgaacccaggaggcggaggttgcagtgagcagagatcgcgccactgcactccagcccagaagactccatccatctcaaaacaaaacaaaacaaaacaaaaacaaaatagttataccagacaaattgttgtctagctggggagagggattcctgacacttcctactgtgccattttccctaatgtcactctgagcctttatgttatagaagggagcagaccatgaggatgcctggtgcatggctttgagggtgtgcacactgacatttatatgtgcacacaaatatgggccgttgtcacaggccagcttgttagacggtggctgtgccatattgggggtgataggaaggggtacaattatgtgtctgtgcatgtttgtgtgtgtcagtgtgtgttcatgtgaggtgataggtgttgctctgtgtttgtacctgcataagtgtacttctgtttgcacctgtgattatacctattctgtgaaccttggagtatgttcatctgggggtacacctaaaactgtgttccggtgtaactgtacagtgcacatacatcttgagggtacccctgagtgtgtgtgtctgtgcatgtccttctctatatgtaccttgtgtgtgacctctgagcatgtacatctctgtgtatattttgtgtacttgtgtgcatgtacctctgtgtacctctaagcatgtatctacgtgtatatctctgagtgtcccactgagcacatccctttgagtgtgtaactgcatgtgtgtctctgaacatgttcctctgtgtgttcctctgatcatggacctctgaacatgtgccttttagcatgtacctctgtgtgtaccttcgagagtgtgagctggattgagccctttaggggtgtgcatagcgaaccaaagctcactgaccctcctccactcctaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtgagttcctggcccgccccaaaccccagcctactccctgtttgagtccctccagtcctctccagtcccctcttcctgatgttctatccctgtcctgctgccctgctgccttgctgccgtatgcctggggagggctgcgtgggggttgggccacgagaaggacccaccaccctgcccagctggccttttcacccttcctcccacctgccccttaggtcttacctggccatccctgcctcctcctgggtggatgacttcattgactggctgaccccgtcctcctgctgccgcctttatatatctggccccaataaggacaagttctgcccctcgaccgtcagtgagtgtggggccatggggactcactgtccaccacagctcgggcaaactgaggcaacagaaaggagaggactggagaggctccctcaacctctcccacgcatcctgcagggtctgtcgggggcatgggtgcagatgtggcctgagggacaggcactctgtgagaagcacctgtgtgggtgaccgtgctggcccgtgggcatcacacatgtatactgctgtgtactgtgcccccattttcagagcacatggtgctcccgggtggcagggcagtggggagtcaggaggggagagctgctgaggttagcacatggccctgccgcccaaagcagtggcatttgtaggtggagaggcctttgtggggcctgtttttctgccccaaacttcctttccccttctgcctgtaggtgcccacagtttctatagccaagaggagaacttctcccacaaatgacaaatgcaaatccccctagaagcgactggttgaggctggagtgcccaggacctttgatgggattgttggggaaggaggggcacaaagcaggagctgctggccctggggtgtcactgcccagacccctgctttctctgcagactctctgaactgcctaaagaactgcatgagcatcacgatgggctctgtgaggccctcggtggagcagttccataagtatcttccctggttcctgaacgaccggcccaacatcaaatgtcccaaagggtaagcttgggagggccttctgctggggaggacagacatgtgggacacaggatggggttgaatatagagaggcaggaggaggctatcaggggcctctctggggtggctgtgggctgggcagatgaaagaagcttcgtccctggctaagcctttgccctgaccttcttgcagcggcctggcagcatacagcacctctgtgaacttgacttcagatggccaggttttaggtaagcatggccttgcctggaggggaggacataaatcggttgctctggagggcccccgaaaaccccagggaacagcctgtcacatgttgtctccctcctttgtcaggaggttctcactgcgctggccctgtcagcaggggtcttgtttcccagctccacatctcagacttcaccccttctctcactcccaagtccatggtcagtgctaagtttgtggaattgattcagcagttgataccatacttgggagttctccacaccctggctaagcacctttcttaccagcacaaattacacccaaagggcagctggttaaatgaattaggatgcttggcacagcacaatcctagcagtcatttaaagtaacaagaggctgggcgcctgtaatcttagcactctaggaggccaaggcgagaggatctcttgaatccaggagtttgagaccagcccaggcaacagtagggagaccctcttttttttttttcgagacggagtctcgctttgttgcccaggctggagtgcagtggtgcaatctcggctcactgcaacctccactttccgggttcaagcgattctcctgcctcagcctcctgagtagctgggactataggagcataccatcatgtctgggtaatttttgtattttcagcagagatggaattgcaccacgttggccaagctggtctcaaactcctgacctcaggtgatatgcctgccttggcctcccaaagtgctagtattacaggcatgagccactgtgcccggcctcctctacaaagtaaaatttaaaaaattgcccgggtgtggtggcgtgtgcctgtagttccagctattcagaaggctgggcgggaagaatgcctgagtctgggaggttgaggctgtagtgaactgtgatcgcaacactgcactccagcctgggcaacaaagtgagaccctctctcaaaaaaaaaaagaaagaaaaaagtaacaagagagatgcagttggactgacaggaaaaggacccacaacatgctgtcagcttatacagcagatggcagaacaagacagccatctgtgtaaaggagctggccatagctccgtgcagacatgctcggtgtaggggccctaagggagctcgtgctggagatggacatgggggtcgtcggtgggtgggggagtttttgaaggatgatctcactttgtactgaaataattcatagtttgaactgctggctgaaagctgcctcaagttcgctcaccccacccttccagctatgaagttcccatgtttccagaagggcaatgcaccctgcccagccctggtagctgagcacaacaggctctgtgaggccagtgtggtggggctggtgtggacagatgggagtggatgtgtcagtcagggaatgaggagcagggcctggaaggagcacacagtagagccaagcccccataaccgggggcaagtctgcaccatctctgacctttgtcttcttgtgtgtgcactaggttagtctagagcagcacttcccaaaatgaggtcccccagccagcagcatcagcataacctggaaattgttcaaaatgaagttccagctaggtgctgcagctcacgcctataatcccagtacgttgggaggccaaggtgggaggatcacttgagcccaggagtctagtctgtctgagaccagcctgggcaaaaaagccagatattgaaagaaaagaagagagaagaaaaggaaagaaaagaaaagaaaagaaagaaagggagaaagagagagagagaaagagagagaaagagaaagaaagaaagaaggaaggaaggaaggaagaaaaagaaagaaagaaagaaaaagaagaaacgcaagttctcagccctcacccaagactttgcagaccccgaattgctgggctgggctgggcatttgtgtgtgaactaccctccaggtggtcagaggcctggtgggaagttctccaggcacctcccctgctctgagattgtatgtatccaagaacatttctcttcttttttctccacacctatgtagcactattgtttctttttcagatacacatgctcactgtacacaataaagaaataactttttttttttttttgagacacagttgccattctgtcacccaggctggagtacagtggcacaatctcggctcactgcaacctctacctcctggattcaagtaattctcctgcctcagcctccctagtagctgggattacaggcacatgccactatgctcagctaatttttgtattattaatagaggcagagtgtcgccaagaaacaacctttttgggccaggtgcggtggctcacacctgtaatcccagcagtttgggagaccgaggccggcgaatcacttgaggtcaggagtttgagaccagcctggccaacatggtgaaaccctgtctctactaaaaatacaaaaattagccaggcatggtggcatgcacctgtaatcccagctacttgggaggctgaggcaggacaatcacttgaacccgggaagcagaagttgcagtgagccaagatcgcaccactgcactccagctgcggtgacagtgagactctgtctcgaaaacaaaaacaagaacaaaaaaccctttattgtataaaggtcttaataaccttaatttcttcttttttttttttgagatgggatcttgctctgttgcccagctggagtgcagtagcatgatctcagctcactgcagcctctgcctcctgagttcaagaattctcctgcctcagccccccaagtagctgggattacaggggtgtgccaccacgcctggctaatttttgcatttttagtagagacagggtttcaccatgttgggcaggctggtcttgaactcctgacctcaggtgatcgacctgccttagccttccaaagtgctgggattacaggcatgagccaccacacccggccaataaccttaatttcttaaaagtcattaagaaataacctttatctggcaggagccctaagccacagctctaataatccaaccgttctcatttttctgtcttcctttctagtcctttcctataggaatatgcaaattaaaaaccaattaagttaattttaaaaatccaatgcatatcttgaaaccatacagagaagaatctcggttcactagggagatctctgtaggcttcactcatcaaaggtcaggcctgggtctcccacagcagtggggccagctatggagtttgcagggctggtgcaaaacaaaaatatgggcctcttgcacaaaatttactaagaatttcaaatggtggtggcagagccctgaaccccgcttgatcacatgcctgtgccactgcgtctgcggtgttctgaagttgtcctggaaagggctctgacctttgcccttccatcttctgtgtgccatggctgtccagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgtgaggacctgagtggctgggctggagggaggtggggtatggttgctggagactggaggttagggtggagggcttgcaaggagttgcatgagatgaggaccagttttaggtcaggaggctctggctgcagccttgggcctatttcttaggctggtttgtaccccaatataagcctgcctgaccctcagcattctccttctgaagtggggtgtcccacccaccatgagggccccagaggcctgagcctgtgaccatgctctgtgctctggcaggatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggtaacccagcagacacaggcaccagggggcctctggaggggtggttggggatccagcctcatagaatactcctagttcttttttgtttctttttttagaggcagggtcttgctctgttgctcaggcttgagggcagtgacatgatcacagctcactgtagcctcgaacccttgggctcaagcgatcctcctacctcagcctccaaagtagccaggactacaggcacgtgccactgcgtccagctaatattttaatttttgttgtagagacagggtctcactttgttgcccaggctggtctcaaactcctgggctcaagtgatcctctcacctcggcctcccaaagtgttgggattataggcatgagccactgcacccggccaaatactcccagttctgtctagaatctagatgcctgccccacgctggtcctggtggaggcctcatctccctagttccttccccacctctgcctttcttggcttatgccccctctctgcccataggcggtgggcatgtctgtggagtttgtgtcccacattacccgctcctttgccatcagcaccaagcccacctggctggagagggccaaagaggccaccatctctatgggaagtgcggtgagtggagaggagtgggccaccctgtgccccactcgacaccctgtgccctgcctgatgccctgtgccctgcctgatgccctgtgccctgcctgacacctggctctgaaccccccaggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtgggtgagtgcccaggcctgttcctaccagactgtcatgattatgctgacgacaacagtaacagtgcatgctcaccacaaaagctcaggaagtgcaaacgagccatgggcagatgtcagaagccaggactatgaccatgtggcaattctgtcttggaagctactattattcatttaatgtgctgtgaacatctttttttgtcagctatgtatgtctcaaacaacgtttctgtggccctgtacactgtggatcttcactgcactgctgttggacttttaagcatgcccttcagcaagaaatatattttacacagagaggtgacatgcacgggcacacatagacatgcctgcctaaaacaaatgcttcactaaataatattaatacttcctttatacatgtgaagcattctgatattgctggttccattctattattattattaatattttttggagacagggtcttgctctgacacccaggctggagtgcagtagcatgatcacagctcactgccaccttgacttcccaggctcaagtgatcctcccacctcagcctcccgagtagctgggaccacaggtgcacaccaccatgcccagctaattttttattttttgtagagatggggtctccctatgttgcccaggctggtctcaaactcctgagctcaagtgatccaccatggccttccacagtgctaggattacaggtgtgagccactgcgcttggcttttattttactttaaatttgttatttattttattttactttacattattttatttttattttttgagatggagtctcgctctgttgcccaggctggagtgcagtggtatgatctcagctccctgcaacctctgcctcccaagttcaagccattctcctgctttagcctcccaagtagctgggattacaggtgcgcaccaccacgcctggccaatttatttatttattttttatttttagtagagacggggtttcaccatgttgggcaggctggtctcgaactcctgacctcaggtgatccaaccgccaaggcctcccaaagtgctgggattacaggcgtgagccactgtgcccagccctatcattaatttgtttttaattattttaattatttttatttttattatttttagacagagtctctctctgttgcccaggctggagtgcagtggcgcaatctcagctcactgcaacctctgcctcctgggttcaagcgattctcctgtctcagcctctcgagtagctgggatatcggtgtatgccaccatacctggctaatttttgtatttttattggagacaggtttcaccatgttggtcaggctggtctcgaactcctgtggcctcaggtgatccatctgccttggcctcccaaagtgcagggattacaggcgtgggccaccgcacccggtctcattaatattttgaaatgctggccaggagtggtggctcatgtttgtaatcctagcactttgggaggctgaggcacatggaagctcaaattgagcctcccaggatgaaggtgtttctggctctcagggtgggcaagctgggaggagttcaattttacctcccaccagatggtaataatattattagaggacatttatagaggggtgtgtttgtgcatcaacatatgtgtctgtaattctcttactacccccgaggcaggtattattatccttcccattttacagatgagggaactgagacacctgccccaggttacagacttggtcaaaggtagtaggggttggagcccacacagctctgtggttcctaaccatgtctcttgtggggactccctgaccctcttggaaggagtagagtgtgtgcgctgggggtggtggatgagacataagagaggggcaaggaggagcagtcgtggggtgtgcttggacaaaggatatccagggccttggagctgcaggtggtggctattccttggaggttcccaaaatgcttgggggatggagggaccaggacatccctgaagcttgggctgtgaacatagtgaccctggaaggcacatggcacagatcccccctgggacccttcctgccctgggtttgttgtacagaaccaggaatagcttctcacctgtgtcccctgcccacctctctgactgtggttctctgtctctccgcagggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgccctcctcaaatcctaggggaggccattcccatgagactgcctgtcactggaggatggcctgctcttgaggtatccaggcagcaccactgatggctcctctgctcccatagtgggtccccagtttccaagtcacctaggccttgggcagtgcctcctcctgggcctgggtctggaagttggcaggaacagacacactccatgtttgtcccacactcactcactttcctaggagcccacttctcatccaacttttcccttctcagttcctctctcgaaagtcttaattctgtgtcagtaagtctttaacacgtagcagtgtccctgagaacacagacaatgaccactaccctgggtgtgatatcacaggaggccagagagaggcaaaggctcaggccaagagccaacgctgtgggaggccggtcggcagccactccctccagggcgcacctgcaggtctgccatccacggccttttctggcaagagaagggcccaggaaggatgctctcataaggcccaggaaggatgctctcataagcaccttggtcatggattagcccctcctggaaaatggtgttgggtttggtctccagctccaatacttattaaggctgttgctgccagtcaaggccacccaggagtctgaaggctgggagctcttggggctgggctggtcctcccatcttcacctcgggcctggatcccaggcctcaaaccagcccaacccgagcttttggacagctctccagaagcatgaactgcagtggagatgaagatcctggctctgtgctgtgcacataggtgtttaataaacatttgttggcagaaatggtgttttatgtcacatgtcctaccctggcttcctcctctcggtttaagataatttttgtgaatgacacaaataatacatgtgtgggagagtgatttgtggagatactagtctgtgttttgttctatttctcctccctcttttcaagaaagtagccaggccattgtgtgctcatgccttacaagggcctttgaggagtgggagtaatttctcttcaaactgggagggcacagagcctgagagtcagtcaggagtaggatgtgcagcccctccttttctggaagagactgtgaagtaggcaacacctggaggagctacaggagaaccacggtgcattcaaggagggaagaacccaccgtacaaacaaccagctcccaggagggccccaggccagggcagtgggtggaaatgtcaaggaacattccagatcccctcgagtctttctgccccatgctgggtccagcccttgtttggctgaggggctgctgttgctttgaggctcagagggactgtcagcatgtaaagggaagacaagcaaaaaggggtggaaaggagctggcgtttctggagcctactatctacttttgggtcctcataagagccccatgtgccagcatcattagcccacctttgggagggttgctggctgaccatgatggacaggaggtttggtgaagggacagctacgagggaatagaggctgaggagaaatcgcacaattcaccctgttaaaaactccacaggtgcagaataaacagatagatttgaggaacaaaatagcttttgacagcagacatttcaaatcagaggaaagggtagatccttcagtaaacggtgtgagagtagtgagcaaattatttggatcaaaataaagttatatctatacttcacacaatacacaaaataaaagtacagacagattaaagcactaaacacaaaaatgaaactatacaactatcggaaggaaacacagaagagtatgttataatcttggagggggaaaagtttcctaagcacaaagtccagaagccataaaggtaaacactaaggtatgaccatataataatggaaaacatctgaaaacacacaaaaaattaaagaaagttgaaagacacatatgagctcagaaaaatagttgcaacatatttaacagcaaataaaatcaagaaaacacaaagagtgccaatagtgctcctgcaaacatggtgaacactcctaaaacccactggactttctgtaagaagtgtgggaagcaccagccccacagagtgacacaggacacatttccctgtatgcctagggaaagccatgttatgacaggaagcagaggggctatggtggggagactaagccaattttccagaaaaaggctaaaactacaaagaagattgtgctaagttttgagtgcatgaagcccaactgcagatctaagagaatgctggctattaagagatacaagcattttgaactgggaggaggtaagaagagcaagggccaagtgatccagttctaagtgtcatcttttgttttattatgaagacaataaaatattgagtttatgtttaaaaaaaaaaagaatatacaaagagagtccaggtacggtggctcatgcctgtaatcccagcactttgggaggctgaggcaggagaattgcttgaggccaggagttcaagaccagcctaggcaacatagcgagatactgtctctacaaaaagtttaaaagttagccaggctagctatttggaaggctgaggtgggaggattgtttcagctcgagtttgaggctgcagtgagctatgatggcaccactgtactccagcctgagtgaaagagtgagcttctgtctcaacaaaaaaaaaaaaaaaaaagaatatacaaagagaggaaggagtgcaggggggaggtctgggttatgtggctaaccttcccattagaaacaagacattctagctaaaataaatcttagccgtgtgtgtgtgtgtatgtgtctgtgtgtgtgtatgatgcatacaagtttagggtgttttaaccttcttgataaattgagacttttatagtttgaaatgactataaaaatatccctttttatctctagtatttatttttgtctgtttaagagatggggttctcactttgttgcccaggctggtcttgaatacttggcctcaagggatcctcctacctcagcctcccaagtacctggaattacaggtatgagccaccatgccagtcctatctgtagtatttgttcaactgtataatgttattatacacacacacacacacacacacacacacacacagacacacacacacatataaaataacatacggttgaacaaattttatacttaatagtcaaacattgaaaccctttcccctgagattgggaatgagacaaagttgcccacttttacccaacattgcactggaggtcttagccattgtaataaggcaagaaaaagaaactaagtttataaggattagaaataaataaaattgacatcattcacagataacataaatatgtataaaaaagattcagtctgggtgcagtggctcatgcctgtaaccccagcaatttctgaggccaaggcaggaggatcacttgaggccaggagttcaagacatagcaagaccccacctctacaaaaaaaaattttttttaaagatccaaaagaatctatatataaactattggaattactctaacaaaaggtggtcaagaaaactatgaaaaataataactttgtattttaatttgtataatattgagagaaattaactgtcaaaagaaatggaggaatataccatgaattgagggctctatactacagagatgtcaattctcttcaaattaattactagtttcactgtaatttcaataataaccccagaaaattttttgtggaaactgataagctgattcaaaaattcatatagaaccacaaaagatgaaaattcacgaaagcaatcttgaagaaaaacaaagtcagagaacttacactactagaaatcaagataatataaatatatagaaataaagatagtgagattttggcacaaggaagaacaaatagaaaaatggaaagaatagaaagtccagaaacagatgatacccacaaggacacatgatttatgatggaggaggcatgcagagcattgggtaaaggaggtttttcaatgtaggatgctgacctagttgggtatccacacagaaagaaatgaatcatgaccctctcccccaagatacacaaaaatcagttcctgatagattgtcaatctaaatgtgaaagataaaatgatagagttctaaaaggtaacataaaagagtatccccaagactgaaataggaaaaacttttcttaggaaacaaaagccttacttatagagaaaaagattgataaattgaactgtattggaataaaaaaaaacttctgttcttcaaaagacatccttaggaaagataaaattcaaaccatagagaggaaaagatatttgcacatatctgaaatacacacatatctgagaaagggcctgtgcttagaatgcataaaaaatctcctacaactcagcaagaaaaagacagacaaccaaaagaaaagctaggctggctactcaaataagcaaatggccaatacaagttcctcaattttgtcagtcaccagagcaaggctgagtaaaagcacagtgagagttcttcctcttctcttccctcacaatttggcctacaggccatggggtaaggtggggccaggcagcacatgtggggtgtcagaatccaggtggtgtggggagcgtttccacattggatctgagggaggagaggagggcattccacacagaataggaactacataggcccagtatggggctaagatgtcagaactgagctctgatgtgcctttctccatgagcagagggactggatgctggagatggagggtggaggaaaggttcagagccatctagagatggcaattcagaggaaatgggagggcagatagtctcactcttcacagtgaggcagagtttccaagctggttttgtcactcctttgctgggcctctttgggtaacatatttgacttatctgggctttagtttcttttttgcttttttttttttttgagacagagtctcactctgttggccaggctggagtgcactggtgtgatcttagctcactgcaacctctgcctcccgggtttgagtgattctcctgcctcagcctcccgagtagctgcaactacaggcgcctgccaccatgcctggctaatttttgtatacagatagggttttgccatgttgaccaggctggtcttgaactcctgacccgaggtgatatgcctgcctcagcctcccaaattgctaggattacaggtgtgagccaccacacctggcatgggtttggtttctttacctgtaaaaactgggatagtttagctgggcacagtgatgctaattgttgtcccagctacttgagaggctgagatgggaggatcacttgagcctaagaatcgcaggtcagcctgggcaacatagcaataccccatctgtgaaaaaaaaaattagtggctgagcacagtggctcactccagcaatcccagaactttgggaggccaaggtgggaagattacttgagcccaggagtttgaaactggtctgggaaacacacagagaccacaatctctgcattaaaaaaaaaattagctgggttggtggcactcacctgtggtcccagctacttgggagggtgaggtgggaggataatttgatcccaggaagtggaggctgcagagagctgtgatcatgccactgcactccagcctgggtcacagagtgagaccctgtctcaaaaaaaaaaaaaaaaattaggaaaatttgccctgactccccacgttttttttaaaggatgaaatgagatattatatgtgaaagcatctagtacttgtgacatagtaggtgcttaaaaagtgtttccacttcacttctgcctaaaacccagttcagttcctgagttccagatatctaactgtgatgagaagagacgcagccagaggtacctcaaagatagcaacacccccctccgccccgatacctgatgtactgaagtcagaaatttaaaaaaaaaccttgttcttccttcagttttaagttcagtatactgatgaactatcggtcacatttgacgatttactttaaaaataaacaggcttccaaattaacctacttatatggtttgtctgtgtcgccacccaaatctcatcttgaattcccacccgttgtgggagggacctggtgggaggtaattaaatcatgggggcaagtctttcctgtgctgttctcgtgatagtgaataagtctcaagagatctgatggttttaaaaagaggagttcccttgcacaagctctctctctttgcctgctgccatccatgtaggatgtgacttgctcttccttgccttccaccatgattgtgaggcttccccagccacatggaactgtaactccaattaaacctctttctcttgtaaattgcccagtctaggctatgtctttatcagcagtgtgaaaacagactaatatacttaccttggaaaggccttgtgatccatggtgacatcttgtccctaaggaaagcatcttaccatgagttcctcaaattgttgatgtactgattaatgtgtaaccctctgacactgggaagaacactgatttatttctgaatcataaagttttattgattgtcttgcatgtagacattttagcttgtatgttgcaatctgtatccaacaattgtaacctctgtattgtaccctcaaatgaaagaggaaaaaactcttgtatgaggagtcccctcccttctcctaaactttcctataaaagccttctaccttgtaacagactggaacattcctaacattgttggtgtgtttcctaagcggattctcacatttggcttcaaataaaccttgatcaaattagtgctgcctcaacagccttaatttcaatcaatagtacaagcctctgtttttctatttaatcactactttaaaggtaacctttggaaaatatttaggctctttacaaatttaattaattgaacatattttaactgcatttataaaggtaatagtctccattttcttcctaaatactctgcataagaaacaaaatcttcccatatacttaactcttttaaacctaataaattaaatttatggaatatcattaatataaagtttttatagatgttgtaacactgcacatagatttagcaacatttcaatttacaatcttaagcttatatgaaataccattttaaattggaattatacaattcttacactaatagaccaaatactttaaatgttacaagcatataaaatacgaaatatacaaaaatttccccccatcacacaaatattcttactaaggttttgcttctttgaaacctttctatacacattgtattagtctgttttcatgctgctaataaagacataccccagactgggtaatttataaaggaaagagttttaattgacttatagttcagcatggctggggaggcctcaggaaacttacaatcatggcagagggggaagcaaacatgcccttcttcatatggcaccagtggagagaagaatcagtgcccagtgaaaggggaagccccttataaaaccagcagatctcgtgagaactaaatcactaccacaagaacaggatgggggaaaccgctctcatgattcaacgatctccacctggtccctcccacaacacatggggattatgcaaactgcaagtcaagatgagatttgggtggggacacagtcaaaacctatcaacctaacatccttttcctctccccttccttccttcctcccttccttccttccttccttccttccttccttctttccttccctccctccctccctccccctctctctctttttttctttttcttttctttctttctctctctctctctccctccctccctcccaggctggaatgcagtggtgcgatctcggctcactgcaacctctgcctcccagggttaagctatcctcccacctcagcctcctgagtaactggtgggactacaggcgtgtgacaccacacccagctgatgtttttgtatttttagtggagatggggtttcagtatgttgtccaggctgtccatacccatttttaagtgagttataaatggggttcaaaggtcatactccccttgaggaagacaatcatcatctcagataaccaaggttgcctatgcagtaaggaagaagtaagtcatcattccgggtaactaaatttacctaagaccaaagacatcagctgagagtgagacctggagtctcaggcatcgggagtagttatctcactgctaactaagtttacatggtgagtcaaaagacccagaatacccaacacaatattgaaggaaaacaaagtcagaggactaacactatctgacttctagacttactataaagttatagtaatgaagacagtgaaagaactggtaaagaacagataaataaatcactgtaacagaatatagagtctagaaatagacccaaataaatatagtgaagcaaaggtagactttttttttttttttttttttttgagacagagtctctctctgtcacccaggctggagtgcactggtatgatcttggttcaatgggacctatacctttaccatgagaatcactgggttcaagtgattctcatgcctcagtgtcctgcatagctgggactaaaggcctgcaaacatgcctggctaatttttgcatttttagtagagatggggtttcaccatgttggctaggctggtctcaaagtcctgacctcaggtgatccacccgccttggcttcccaaagtgctgggattacaggtgtgagccaccatacccagccaaagggcagtctttccaacaaatgatacagatacaactggacatctatgtgcaaaaacataaatttagacacagactttgcacccttcacaaaaactaactgaaaatggatcatagacctcaatgtaaaattcaaaactataaaactcctaaaagacaacatagggtaaaacctagatgaccttgggtgtagcgaccttttgatacaacaccaaagacataatccatgaaataaataactgataaactgtaattaataaattttttttagcagtaatagaatgatgagtgttatttcattaaaatttaaaacttctgctctgcaaaagacaatgtcaagaagaagaagacaatggccaagtgcggtggcttatgcctttaatcctagcactttggaaggccaaggcgggtggatcacttgaggccaggagtttgagaccagcctggctaacatggtgaaaacctgtctctactaaaaatagaaaaattagctgggcgcagtggtgcacacctgtaatcccagctacttgacaggctgatgcacaagaatcgcttgaacccaggaggcagaggttgcagtgagctgaaattgtgccactgtactccagcttgggcaacagagcgagactctgtctcaaaaaatatataaataaataaaatttaaaaaggatgagaagacaagccactgcctgggagaagatattagcgaaagacacatctgtgctggcttcagcagcacacatactaaaattacaatggtacagagaagattaccatggcctgtgcacaaggatgacatgcacatttgtgaagtgcttcagaatataaaaaagaaaaagatctatccgataaagaacttttatttaaaatctaaatggactctccaatacaataataagaaaacaaataactcaattaaaaacttagccttaccaaagaagatgtacagatggcaaacaagcatatgaaaagatgctacatgtcatatatcatcagggaaatgataattaaaacaacaatgtgatactgctacacatctattagaatgtccaaaatctggaacactgacaacatcaaatgctggtagggatgtggagaagcagcaactctccttcattactgataggaatgcaaaatggtacagccactttggaagacagttcttcagtttcttctaaaactaaatatatcttaccatatgatccagcaatcacatttcttggtatgtacccaatggagttgaaaacttatgtctacacaaaaaccaacacatgggtgttcatggcagccttattcataattgtcaaaacttggaagtaaccaagatgtccttcagtaggtgaatgggttaatccccacaatggaatattattcagcattaaaaacaaatgagctatcaagctaagctatgaaaagacatggaggggccgggcacggtggctcaagtctgaaatcccagcactttgggaggccgaggtgggcagatcacaaggtcaggagtttgagaccagcccggccaatatggtgaaaccctgtctctactaaaaatacaaaaattcgccgggtgtggtggcaggcgcctatagtcccagctactcagatggctgaggtaggagaggagattcacttgaatctgggaggcagaggttgcagtgagccgagatcacaccattgcactccagcctgggcaacaagagcgaaactccatctcaaaaaaaaaaaaaaaaaaaaagaaaaagaaaaagaaagaaaagaaaagaagtggaggaacttcaaatgtatactactaagtggaaaaagcaaatctaaaaagtctacatctgtctgattccaactatatgacattctgtaaaaggcaaagctataaacacaataaaatgatcggtagtttctagggtttggggttaggggagttgaatgggcagagcacaaaagatttttaggccagggaaaccactctatatgatattataatcatggatgcatgtcattatacatttgtccaaatccatagaatgtacaacaccagagtgagccctaatgtaaactaaggattctgggtgatattgtaacaaatgcaccattaattgtaacaaatgtatcattttgtaccttctgatggggaatgttgagaatgagagaggctatgcatgtgtggaggcaggagtgggtatatgggatatctctgtatcttcctctcaattttgctgtgaacctataactacctaaaaaagtcttttagaaagcccagtagttttttgcttctctttatgggttggtttccttctctcaagtgaaaaatgggcttcctccatgtagcagatgatatggcttctctcatcccagagaagagagttctttcttgtcaattacagccagaaaaatctccaagaaggatttagatggtcctagtttgctccctcccatccctcttcctttggatctcagatcagaagtgacttctactgggatgctgccctgttaccccagtcttggtcgggtccctgttatgtgctcccactataccatatccttctccttcctagtcttcatcacagtttgaagatgaaaattcattggtggggttacatggctcccccatgtctgattcctcctctaaactgtaagctatagggggcaatgactttatttttttgcttaccattgtgtttctagcacctagcatctggcacataggcacacaataaatatccattaaataaatgactgaaataaacagagggctcttttgctctgattactctgaagagcaattattacatagcagtgacagcttagtgtattctcagaaaatattcttttgttttaaaaccacttatttttctggccaggcatggtggctcacgcctgttatctcagcactttgggaggccgaggtaggcggatcacaaggtcaggagatcgagaccatcctggataacatggtgaaaccctgtctctactaaaaatacaaaaaaatgagccgggcttggtggcgggcgcctgtagtcccagctactagggaggctgaggcaggagaatggcgtgaacccaggaggtggaggttgccatgagccaagatcgcaccgctgaacttcagcttgggcgacagagcgagattccatctcaaaaaaaaaaaatttttttttctgataataaacacaacagactgggcacagtggcgcatacctgtaatcctggtacattgggaggccaaggtgggaggatcacttgagtccaggagttcaagaccagcctgggcaacattgtgagacatcatctctatttaaaaacaaacaaacaaacaaacaaacaaacaaacaaacactccttaaatccccacacacttatgacagaataattgtaagacaaagaaaagtacagttaagaaaacaaaaaacaaaaattacttatatctgtaaccc

SEQ ID NO: 21 - DNA, 5092 bp, Homo sapiens
cttggctgttcctgaggcctggcctggctccccgctgaccccttcccagacctgggatggcggaggccggcctgaggggctggctgctgtgggccctgctcctgcgcttggcccagagtgagccttacacaaccatccaccagcctggctactgcgccttctatgacgaatgtgggaagaacccagagctgtctggaagcctcatgacactctccaacgtgtcctgcctgtccaacacgccggcccgcaagatcacaggtgatcacctgatcctattacagaagatctgcccccgcctctacaccggccccaacacccaagcctgctgctccgccaagcagctggtatcactggaagcgagtctgtcgatcaccaaggccctcctcacccgctgcccagcctgctctgacaattttgtgaacctgcactgccacaacacgtgcagccccaatcagagcctcttcatcaatgtgacccgcgtggcccagctaggggctggacaactcccagctgtggtggcctatgaggccttctaccagcatagctttgccgagcagagctatgactcctgcagccgtgtgcgcgtccctgcagctgccacgctggctgtgggcaccatgtgtggcgtgtatggctctgccctttgcaatgcccagcgctggctcaacttccagggagacacaggcaatggtctggccccactggacatcaccttccacctcttggagcctggccaggccgtggggagtgggattcagcctctgaatgagggggttgcacgttgcaatgagtcccaaggtgacgacgtggcgacctgctcctgccaagactgtgctgcatcctgtcctgccatagcccgcccccaggccctcgactccaccttctacctgggccagatgccgggcagtctggtcctcatcatcatcctctgctctgtcttcgctgtggtcaccatcctgcttgtgggattccgtgtggcccccgccagggacaaaagcaagatggtggaccccaagaagggcaccagcctctctgacaagctcagcttctccacccacaccctccttggccagttcttccagggctggggcacgtgggtggcttcgtggcctctgaccatcttggtgctatctgtcatcccggtggtggccttggcagcgggcctggtctttacagaactcactacggaccccgtggagctgtggtcggcccccaacagccaagcccggagtgagaaagctttccatgaccagcatttcggccccttcttccgaaccaaccaggtgatcctgacggctcctaaccggtccagctacaggtatgactctctgctgctggggcccaagaacttcagcggaatcctggacctggacttgctgctggagctgctagagctgcaggagaggctgcggcacctccaggtatggtcgcccgaagcacagcgcaacatctccctgcaggacatctgctacgcccccctcaatccggacaataccagtctctacgactgctgcatcaacagcctcctgcagtatttccagaacaaccgcacgctcctgctgctcacagccaaccagacactgatggggcagacctcccaagtcgactggaaggaccattttctgtactgtgccaatgccccgctcaccttcaaggatggcacagccctggccctgagctgcatggctgactacggggcccctgtcttccccttccttgccattggggggtacaaaggaaaggactattctgaggcagaggccctgatcatgacgttctccctcaacaattaccctgccggggacccccgtctggcccaggccaagctgtgggaggaggccttcttagaggaaatgcgagccttccagcgtcggatggctggcatgttccaggtcacgttcatggctgagcgctctctggaagacgagatcaatcgcaccacagctgaagacctgcccatctttgccaccagctacattgtcatattcctgtacatctctctggccctgggcagctattccagctggagccgagtgatggtggactccaaggccacgctgggcctcggcggggtggccgtggtcctgggagcagtcatggctgccatgggcttcttctcctacttgggtatccgctcctccctggtcatcctgcaagtggttcctttcctggtgctgtccgtgggggctgataacatcttcatctttgttctcgagtaccagaggctgccccggaggcctggggagccacgagaggtccacattgggcgagccctaggcagggtggctcccagcatgctgttgtgcagcctctctgaggccatctgcttcttcctaggggccctgacccccatgccagctgtgcggacctttgccctgacctctggccttgcagtgatccttgacttcctcctgcagatgtcagcctttgtggccctgctctccctggacagcaagaggcaggaggcctcccggttggacgtctgctgctgtgtcaagccccaggagctgcccccgcctggccagggagaggggctcctgcttggcttcttccaaaaggcttatgcccccttcctgctgcactggatcactcgaggtgttgtgctgctgctgtttctcgccctgttcggagtgagcctctactccatgtgccacatcagcgtgggactggaccaggagctggccctgcccaaggactcgtacctgcttgactatttcctctttctgaaccgctacttcgaggtgggggccccggtgtactttgttaccaccttgggctacaacttctccagcgaggctgggatgaatgccatctgctccagtgcaggctgcaacaacttctccttcacccagaagatccagtatgccacagagttccctgagcagtcttacctggccatccctgcctcctcctgggtggatgacttcattgactggctgaccccgtcctcctgctgccgcctttatatatctggccccaataaggacaagttctgcccctcgaccgtcaactctctgaactgcctaaagaactgcatgagcatcacgatgggctctgtgaggccctcggtggagcagttccataagtatcttccctggttcctgaacgaccggcccaacatcaaatgtcccaaaggcggcctggcagcatacagcacctctgtgaacttgacttcagatggccaggttttagacacagttgccattctgtcacccaggctggagtacagtggcacaatctcggctcactgcaacctctacctcctggattcagcctccaggttcatggcctatcacaagcccctgaaaaactcacaggattacacagaagctctgcgggcagctcgagagctggcagccaacatcactgctgacctgcggaaagtgcctggaacagacccggcttttgaggtcttcccctacacgatcaccaatgtgttttatgagcagtacctgaccatcctccctgaggggctcttcatgctcagcctctgccttgtgcccaccttcgctgtctcctgcctcctgctgggcctggacctgcgctccggcctcctcaacctgctctccattgtcatgatcctcgtggacactgtcggcttcatggccctgtggggcatcagttacaatgctgtgtccctcatcaacctggtctcggcggtgggcatgtctgtggagtttgtgtcccacattacccgctcctttgccatcagcaccaagcccacctggctggagagggccaaagaggccaccatctctatgggaagtgcggtgtttgcaggtgtggccatgaccaacctgcctggcatccttgtcctgggcctcgccaaggcccagctcattcagatcttcttcttccgcctcaacctcctgatcactctgctgggcctgctgcatggcttggtcttcctgcccgtcatcctcagctacgtggggcctgacgttaacccggctctggcactggagcagaagcgggctgaggaggcggtggcagcagtcatggtggcctcttgcccaaatcacccctcccgagtctccacagctgacaacatctatgtcaaccacagctttgaaggttctatcaaaggtgctggtgccatcagcaacttcttgcccaacaatgggcggcagttctgatacagccagaggccctgtctaggctctatggccctgaaccaaagggttatggggatcttccttgtgactgccccttgacacacgccctcctcaaatcctaggggaggccattcccatgagactgcctgtcactggaggatggcctgctcttgaggtatccaggcagcaccactgatggctcctctgctcccatagtgggtccccagtttccaagtcacctaggccttgggcagtgcctcctcctgggcctgggtctggaagttggcaggaacagacacactccatgtttgtcccacactcactcactttcctaggagcccacttctcatccaacttttcccttctcagttcctctctcgaaagtcttaattctgtgtcagtaagtctttaacacgtagcagtgtccctgagaacacagacaatgaccactaccctgggtgtgatatcacaggaggccagagagaggcaaaggctcaggccaagagccaacgctgtgggaggccggtcggcagccactccctccagggcgcacctgcaggtctgccatccacggccttttctggcaagagaagggcccaggaaggatgctctcataaggcccaggaaggatgctctcataagcaccttggtcatggattagcccctcctggaaaatggtgttgggtttggtctccagctccaatacttattaaggctgttgctgccagtcaaggccacccaggagtctgaaggctgggagctcttggggctgggctggtcctcccatcttcacctcgggcctggatcccaggcctcaaaccagcccaacccgagcttttggacagctctccagaagcatgaactgcagtggagatgaagatcctggctctgtgctgtgcacataggtgtttaataaacatttgttggcagaaaaaaaaaaaaaaaaaaaaaaaaaaaaaaaaaaaaaaaaaaaaaaa

SEQ ID NO: 22 - protein, 1359 aa, Homo sapiens
MAEAGLRGWLLWALLLRLAQSEPYTTIHQPGYCAFYDECGKNPELSGSLMTLSNVSCLSNTPARKITGDHLILLQKICPRLYTGPNTQACCSAKQLVSLEASLSITKALLTRCPACSDNFVNLHCHNTCSPNQSLFINVTRVAQLGAGQLPAVVAYEAFYQHSFAEQSYDSCSRVRVPAAATLAVGTMCGVYGSALCNAQRWLNFQGDTGNGLAPLDITFHLLEPGQAVGSGIQPLNEGVARCNESQGDDVATCSCQDCAASCPAIARPQALDSTFYLGQMPGSLVLIIILCSVFAVVTILLVGFRVAPARDKSKMVDPKKGTSLSDKLSFSTHTLLGQFFQGWGTWVASWPLTILVLSVIPVVALAAGLVFTELTTDPVELWSAPNSQARSEKAFHDQHFGPFFRTNQVILTAPNRSSYRYDSLLLGPKNFSGILDLDLLLELLELQERLRHLQVWSPEAQRNISLQDICYAPLNPDNTSLYDCCINSLLQYFQNNRTLLLLTANQTLMGQTSQVDWKDHFLYCANAPLTFKDGTALALSCMADYGAPVFPFLAIGGYKGKDYSEAEALIMTFSLNNYPAGDPRLAQAKLWEEAFLEEMRAFQRRMAGMFQVTFMAERSLEDEINRTTAEDLPIFATSYIVIFLYISLALGSYSSWSRVMVDSKATLGLGGVAVVLGAVMAAMGFFSYLGIRSSLVILQVVPFLVLSVGADNIFIFVLEYQRLPRRPGEPREVHIGRALGRVAPSMLLCSLSEAICFFLGALTPMPAVRTFALTSGLAVILDFLLQMSAFVALLSLDSKRQEASRLDVCCCVKPQELPPPGQGEGLLLGFFQKAYAPFLLHWITRGVVLLLFLALFGVSLYSMCHISVGLDQELALPKDSYLLDYFLFLNRYFEVGAPVYFVTTLGYNFSSEAGMNAICSSAGCNNFSFTQKIQYATEFPEQSYLAIPASSWVDDFIDWLTPSSCCRLYISGPNKDKFCPSTVNSLNCLKNCMSITMGSVRPSVEQFHKYLPWFLNDRPNIKCPKGGLAAYSTSVNLTSDGQVLDTVAILSPRLEYSGTISAHCNLYLLDSASRFMAYHKPLKNSQDYTEALRAARELAANITADLRKVPGTDPAFEVFPYTITNVFYEQYLTILPEGLFMLSLCLVPTFAVSCLLLGLDLRSGLLNLLSIVMILVDTVGFMALWGISYNAVSLINLVSAVGMSVEFVSHITRSFAISTKPTWLERAKEATISMGSAVFAGVAMTNLPGILVLGLAKAQLIQIFFFRLNLLITLLGLLHGLVFLPVILSYVGPDVNPALALEQKRAEEAVAAVMVASCPNHPSRVSTADNIYVNHSFEGSIKGAGAISNFLPNNGRQF

SEQ ID NO: 23 - DNA, 21 bp, artificial
  synthetic sequence
tggtctttacagaactcacta

SEQ ID NO: 24 - DNA, 21 bp, artificial
  synthetic sequence
tccggacaataccagtctcta

SEQ ID NO: 25 - DNA, 76 bp, artificial
  synthetic sequence
ggatcccgtagtgagttctgtaaagaccattgatatccgtggtctttacagaactcactattttttccaaaagctt

SEQ ID NO: 26 - DNA, 76 bp, artificial
  synthetic sequence
ggatcccgtagagactggtattgtccggattgatatccgtccggacaataccagtctctattttttccaaaagctt

SEQ ID NO: 27 - DNA, 960 bp, Homo sapiens
atctgcagctcagctttggtaatgggggcccattaccaaatgggggtaaaggtcatggcccatcctggtgatagtgagaacccaaggtaggccttgaagattcctatcaggagggagcagaaagtgtgtaccacacccctgggcccaggtggagcagggctgctgctcaaggctcccagccatgctctgtcccttgctaggggtgaccggtgggacaggcctgggcaagggacaagagggagaaggtcggggggaagaggggatgaagagcaaagtgagcaaaggagagtcttccactatctggggtctctgtcaactgtcaggccctagagtgagctgttctttccctttgcttcctggaggaggggacttttgtcactgcgtcactccaccctgcctgcccctccgttatcaggctgttaatattaattaacaacagttgctagggatgacagtgcagagggttcctctgagcccattgctggccctggtcccaagagggggtagggcagagctggggtctgaggctgagccagggagggtgcggaggttcctcggccatgctgagctcctgaggccgggtcccagccagtgcctggtcccatctgtgcctccaggccctggcaccaactccagcagtgttaggggctaatagcgtggtctctcccctagctgactcagccctctggcttcggtcgctttgggaagtgagtggagaccctagcacctgcgtgatgaggctcatctaaagcgggggcctgtggactggggccaaacagtgggagtggtggatcattaaccagcagggctcagcctcattggtccctaacccagtcaggccagggttgtcatcgaaggggaggaggctgccttaatgtgtgttcagcccttggctgttcctgaggcctggcctggctccccgctgaccccttcccagacctgggatggcggaggccggcc

SEQ ID NO: 28 - DNA, 970 bp, Mus musculus
cctgcctaagcttgggcggattcccctctgagcccacccgagcccctgggacactggtggaactcagtaggagcccctccctgcagctgtctcaacaggtagctgcatgagtggccttgaagcaattatcagcaattcagccctggcaatagaggccaaggtcctggcctgtcttggtgatagcaagagcccaaggaaagactggaagtttcctactggaaagaagcagaggatgaaccatgtacctgggcccaggttgggtgggacttgccactcagagcccctaaccagggttgttcagaggactaggccagggccaggaccaagaaagggatagaacgggcatgaggaggaagggtgaagggatccaaggaatctctggtcctgttccctgttaggacatttgtcatggaatcactctcgcttagtgtctctgttatctgggtgctaatagcaactattcagttgctaggatgttaggtgagtctgaacctacccttgatgttgatctgaagaggcgatgcgttagactgcaggttggaggccaagtccaggacagtgttgatattctggatctccaagaagcctccaaggccaaagccaggccagtgtctggtctcgcagaggaacagctctgcatctcttgcccggttggctctaactaccacattagacttcagttgcgtcaaaaaacgaggggaccccagcgccttcactaggaagttgacctcagaaggaggagatggaatggcaccatctgatgtaagggaagagaaaataaattattaaccagtacggcccagtcctattggccccatgacagacgagggttatcactaagaggaggaagctgccttaatgtgcaaactcaggggccagtcctcagcttccccggctgtctccaaggcctggtcctgcttttccttgatcacttcctggctctgggatggcagctgcctggcagggatgg

SEQ ID NO: 29 - protein, 8 aa, artificial
  synthetic peptide tag
DYKDDDDK

SEQ ID NO: 30 - DNA, 23 bp, artificial
  primer
ctatacgaagttatgtcaagcgg

SEQ ID NO: 31 - DNA, 25 bp, artificial
  primer
cttgcacctgacttcctcatataag

SEQ ID NO: 32 - DNA, 23 bp, artificial
  primer
aaagaaggaaagcggccgccagg

SEQ ID NO: 33 - DNA, 25 bp, artificial
  primer
aggaaccgtactgagcgcataccaa